SEQUENCE LISTING (48 sequences)

SEQ ID NO: 1 - protein, 323 aa, synthetic construct
QDVQVFADNPVLGTEVTFEKDENGRIVKIISKNQYRITIYNSVDAGDTPNDASVSLDVDFIDDKNSGEMGAVASINTFIPSGLRYVEGYKYKGVTNPIYKNLSAGMLWPKKYRVEVVNIPIDQATKIITATPNNNIKEKQVSDTISYGFGGSVSADGKKPGGSIEANLAYTKTTTYDQPDYETSQIKKTTKEAVWDTSFVETRDGYTPNSWNPVYGNQMFMRGRYSNVSPIDNIKKGGEVSSLISGGFSPKMGVVLASPNGTKKSQFVVRVSRMSDMYIMRWSGTEWGGENEINQNVPKEYNALMYEDVKFEIDWEQRTVRTI

SEQ ID NO: 2 - protein, 306 aa, synthetic construct
SFNEDNILGTTTKETDEDGKTKTIITVKDRQIESYTSSDSATKENNTSLTVNANFLNDKYSSDLTTIMSLNGFIPSGKQYYFPPNNTWVGNMYWPSQYKTTVSSESLDQTVKIINTTPNNAIRNKDVSTSITYGVGGGVKVEGKTVGANIDANAAFTKTISYEQPDYETVQTQWTTDKTTWETNFTETTDGYTRTSWNTFYGNQIFMENPYTYNVKNNFTPNYKLSPLIAGGFSPSYGVVLKAPKNTKKSIVTVYMSRINDTYQQYWNGFNWVGRDFVDQSYSNWITNSNFKYEINWEEHTVKFIA

SEQ ID NO: 3 - protein, 306 aa, synthetic construct
ASEEDTFAGNVSTETDSNGTEKNIITSNGKQIENYNSTDSNTKKNNSSITINANFIEDTHSSEKTTVLSVNGFVPSGRKFIYPKNNTWEASMLWPSSYKATIVNESIDSPVKITNSTPSNTIRNKEVSSSISYGFGGGISIQGKSAGGSLNGNVAFTKNISYQQPDYETIQTKGTNYEVAWKTNFTETTDGYSRDSWHGIYGNQMFMKSRQYNSRENNFTADYQLSALITGGFSPNYGVVLKAPKNVKKSLIKVALGRESETYTLKWTGFSWTGVNRLDQSNSDWSSNVLFIYELDWENHTLKSIS

SEQ ID NO: 4 - protein, 304 aa, synthetic construct
SEDNIIGTTTQEIDEHGNVKTIITVKNQQIESYTSTDSGTAKNRSTLTVNANFLNDKYSNELTTILSLNGFIPSGRKFIFPKNNTLKGEMLWPQRYSTAVYNIPLDKSVKITNSTPDNTIRSKEVSNSITYGIGGGIKMEGKQPGANLDANAAITKTISYQQPDYETAKTTSTVTGVNWNTNFTETRDGYTRNSWNPVYGNQMFMYGRYTSNIRNNFTPDYQLSSLITSGFSPSYGLVLRAPKDVKKSRIKVVFARRSETYQQNWDGLNWWGRNFYDTKNPDSLSKVTLTFELDWQNHRVTFIE

SEQ ID NO: 5 - protein, 307 aa, synthetic construct
ADFSEDNIIGTTTHEIDEHGNVKTIITVKNQQVESYTSTDSGTAKNRSTMTVNANFLNDKYSNELTTILSFNGFIPSGRKFIFPKNNTWKGEMMWPQRYSTAVYNESLDKPVKISDSTPNNTIRNKEVANSITYGIGGGIKMEGKQPGVNLDANAAITKTISYQQPDYETTKTTSTINGVNWNTDFTETRDGYTRNSWNPVYGNQLFMYGRYTHDVRNNFTPNYQLSSLITSGFSPSYGLVLRAPKDVKKSRIKVVFARRSEIYQQNWDGANWWGNNFYDNKRPNSLTRVTLTFDLDWQNHRVTFIQ

SEQ ID NO: 6 - protein, 308 aa, synthetic construct
ADFNEDNILGTTTVERDEQGNIKTIITVKNRTVESYSSNDTATSNNYDMTATLNATFLNDKYSNNLTTILSLNGFIPSGRKFSFPSNNSWLGSMLWPEQYSIKVMSESLDKPVKITDSTPTNTIRSKEVSDSITYGIGGGVKIEGKTPGVATDVNAAFTKTISYHQPDYETTKVKGTISESLWNTSFTETIDGYNRTSWNPVYGNQMFMYGRYTSNIKNNFTPDYQLSPLIRGGFSPSYGIVMRGSKDVQKSRVKVIFGRTSETYEQSWSGFNWVGRNFLNQSTSDWYASSTFLFEIDWQNHTVKFIA

SEQ ID NO: 7 - protein, 307 aa, synthetic construct
ADFNESNTIGTTTTEKDEDGKTKTIITVKDRQIESYTSTDAATSSNNSSVSINATFFNDRYSDKLTTLLSFKGFIPSGRRFTFPPRNSWLGFMLWPYKYSTTITNISLDKPVKIIESTPSNTIKNKEVSDSIVYGVGGGVKLEGKTPGASLDANAAFSKTISYQQPDYETIKTSGTTQKTSWDTVFTETTDGYTRTSWNSLFGNGLFMVGLYTYDVKKNFTPDYKLSQLITGGFSPEYGLVLNAPKEVKKSQIKVQLSRVSETYRQEWTGFNWFGRNYLDQNTSDWSSTSSLLFEIDWEKHTVKFIA

SEQ ID NO: 8 - protein, 307 aa, synthetic construct
ADYNEDNILGTTTTETDENGNIKTIITVKNRQIETYNSSDSATTSNNSSITVNGTFFNDKYSQELTTILSVSGFVPSGRKFIFPPHNTWVGSMLWPVQYSTTVKNSSKDNTVKISDSTPANTIRNKEVSNSITYGIGGGVKIEGKTPGANLDSSVAFTKTISYQQPDYETVKTVGTTQMATWKTTFTETTDGYTRNSWNPTYGNEMFMYGRYTYDVKKNFTPDYKLSPLITGGFSPSYGIVLKARKNTKKSTIEVFLDRQSETYKQEWSGFNWYGSNYLDQTKPDWSSGAALTFEVDWQNHTIRFIA

SEQ ID NO: 9 - protein, 345 aa, synthetic construct
MLKLKKIFLFTMISVSLCCGFGQDVQVFADNPVLGTEVTFEKDENGRIVKIISKNQYRITIYNSVDAGDTPNDASVSLDVDFIDDKNSGEMGAVASINTFIPSGLRYVEGYKYKGVTNPIYKNLSAGMLWPKKYRVEVVNIPIDQATKIITATPNNNIKEKQVSDTISYGFGGSVSADGKKPGGSIEANLAYTKTTTYDQPDYETSQIKKTTKEAVWDTSFVETRDGYTPNSWNPVYGNQMFMRGRYSNVSPIDNIKKGGEVSSLISGGFSPKMGVVLASPNGTKKSQFVVRVSRMSDMYIMRWSGTEWGGENEINQNVPKEYNALMYEDVKFEIDWEQRTVRTI

SEQ ID NO: 10 - protein, 334 aa, synthetic construct
MKCMNLKKIFLFNTVIISILLVSSPVSASFNEDNILGTTTKETDEDGKTKTIITVKDRQIESYTSSDSATKENNTSLTVNANFLNDKYSSDLTTIMSLNGFIPSGKQYYFPPNNTWVGNMYWPSQYKTTVSSESLDQTVKIINTTPNNAIRNKDVSTSITYGVGGGVKVEGKTVGANIDANAAFTKTISYEQPDYETVQTQWTTDKTTWETNFTETTDGYTRTSWNTFYGNQIFMENPYTYNVKNNFTPNYKLSPLIAGGFSPSYGVVLKAPKNTKKSIVTVYMSRINDTYQQYWNGFNWVGRDFVDQSYSNWITNSNFKYEINWEEHTVKFIA

SEQ ID NO: 11 - protein, 329 aa, synthetic construct
MKLKFALSVLSLSTILLSASPVLASEEDTFAGNVSTETDSNGTEKNIITSNGKQIENYNSTDSNTKKNNSSITINANFIEDTHSSEKTTVLSVNGFVPSGRKFIYPKNNTWEASMLWPSSYKATIVNESIDSPVKITNSTPSNTIRNKEVSSSISYGFGGGISIQGKSAGGSLNGNVAFTKNISYQQPDYETIQTKGTNYEVAWKTNFTETTDGYSRDSWHGIYGNQMFMKSRQYNSRENNFTADYQLSALITGGFSPNYGVVLKAPKNVKKSLIKVALGRESETYTLKWTGFSWTGVNRLDQSNSDWSSNVLFIYELDWENHTLKSIS

SEQ ID NO: 12 - protein, 335 aa, synthetic construct
MIKLKKKNYKIWLSIAVLSAMLTTVPVFANFSEDNIIGTTTQEIDEHGNVKTIITVKNQQIESYTSTDSGTAKNRSTLTVNANFLNDKYSNELTTILSLNGFIPSGRKFIFPKNNTLKGEMLWPQRYSTAVYNIPLDKSVKITNSTPDNTIRSKEVSNSITYGIGGGIKMEGKQPGANLDANAAITKTISYQQPDYETAKTTSTVTGVNWNTNFTETRDGYTRNSWNPVYGNQMFMYGRYTSNIRNNFTPDYQLSSLITSGFSPSYGLVLRAPKDVKKSRIKVVFARRSETYQQNWDGLNWWGRNFYDTKNPDSLSKVTLTFELDWQNHRVTFIE

SEQ ID NO: 13 - protein, 335 aa, synthetic construct
MVKLKKKNHKIWISIAVLSAMLTTVPVFADFSEDNIIGTTTHEIDEHGNVKTIITVKNQQVESYTSTDSGTAKNRSTMTVNANFLNDKYSNELTTILSFNGFIPSGRKFIFPKNNTWKGEMMWPQRYSTAVYNESLDKPVKISDSTPNNTIRNKEVANSITYGIGGGIKMEGKQPGVNLDANAAITKTISYQQPDYETTKTTSTINGVNWNTDFTETRDGYTRNSWNPVYGNQLFMYGRYTHDVRNNFTPNYQLSSLITSGFSPSYGLVLRAPKDVKKSRIKVVFARRSEIYQQNWDGANWWGNNFYDNKRPNSLTRVTLTFDLDWQNHRVTFIQ

SEQ ID NO: 14 - protein, 336 aa, synthetic construct
MKIFRKSISKRIFFCAVLSGLMYTSPVQADFNEDNILGTTTVERDEQGNIKTIITVKNRTVESYSSNDTATSNNYDMTATLNATFLNDKYSNNLTTILSLNGFIPSGRKFSFPSNNSWLGSMLWPEQYSIKVMSESLDKPVKITDSTPTNTIRSKEVSDSITYGIGGGVKIEGKTPGVATDVNAAFTKTISYHQPDYETTKVKGTISESLWNTSFTETIDGYNRTSWNPVYGNQMFMYGRYTSNIKNNFTPDYQLSPLIRGGFSPSYGIVMRGSKDVQKSRVKVIFGRTSETYEQSWSGFNWVGRNFLNQSTSDWYASSTFLFEIDWQNHTVKFIA

SEQ ID NO: 15 - protein, 320 aa, synthetic construct
AIISILLASSPVFADFNESNTIGTTTTEKDEDGKTKTIITVKDRQIESYTSTDAATSSNNSSVSINATFFNDRYSDKLTTLLSFKGFIPSGRRFTFPPRNSWLGFMLWPYKYSTTITNISLDKPVKIIESTPSNTIKNKEVSDSIVYGVGGGVKLEGKTPGASLDANAAFSKTISYQQPDYETIKTSGTTQKTSWDTVFTETTDGYTRTSWNSLFGNGLFMVGLYTYDVKKNFTPDYKLSQLITGGFSPEYGLVLNAPKEVKKSQIKVQLSRVSETYRQEWTGFNWFGRNYLDQNTSDWSSTSSLLFEIDWEKHTVKFIA

SEQ ID NO: 16 - protein, 331 aa, synthetic construct
MLKNSRMVLVLALLANFVVGTPVLADYNEDNILGTTTTETDENGNIKTIITVKNRQIETYNSSDSATTSNNSSITVNGTFFNDKYSQELTTILSVSGFVPSGRKFIFPPHNTWVGSMLWPVQYSTTVKNSSKDNTVKISDSTPANTIRNKEVSNSITYGIGGGVKIEGKTPGANLDSSVAFTKTISYQQPDYETVKTVGTTQMATWKTTFTETTDGYTRNSWNPTYGNEMFMYGRYTYDVKKNFTPDYKLSPLITGGFSPSYGIVLKARKNTKKSTIEVFLDRQSETYKQEWSGFNWYGSNYLDQTKPDWSSGAALTFEVDWQNHTIRFIA

SEQ ID NO: 17 - protein, 323 aa, synthetic construct
QDVQVFADNPVLGTEVTFEKDENGRIVAIISKNQYRITIYNSVDAGDTPNDASVSLDVDFIDDKNSGEMGAVASINTFIPSGLRYVEGYKYKGVTNPIYKNLSAGMLWPKKYRVEVVNIPIDQATKIITATPNNNIKEKQVSDTISYGFGGSVSADGKKPGGSIEANLAYTKTTTYDQPDYETSQIKKTTKEAVWDTSFVETRDGYTPNSWNPVYGNQMFMRGRYSNVSPIDNIKKGGEVSSLISGGFSPKMGVVLASPNGTKKSQFVVRVSRMSDMYIMRWSGTEWGGENEINQNVPKEYNALMYEDVKFEIDWEQRTVRTI

SEQ ID NO: 18 - protein, 323 aa, synthetic construct
QDVQVFADNPVLGTEVTFEKDENGRIVEIISKNQYRITIYNSVDAGDTPNDASVSLDVDFIDDKNSGEMGAVASINTFIPSGLRYVEGYKYKGVTNPIYKNLSAGMLWPKKYRVEVVNIPIDQATKIITATPNNNIKEKQVSDTISYGFGGSVSADGKKPGGSIEANLAYTKTTTYDQPDYETSQIKKTTKEAVWDTSFVETRDGYTPNSWNPVYGNQMFMRGRYSNVSPIDNIKKGGEVSSLISGGFSPKMGVVLASPNGTKKSQFVVRVSRMSDMYIMRWSGTEWGGENEINQNVPKEYNALMYEDVKFEIDWEQRTVRTI

SEQ ID NO: 19 - protein, 323 aa, synthetic construct
QDVQVFADNPVLGTEVTFEKDENGRIVEIISENQYRITIYNSVDAGDTPNDASVSLDVDFIDDKNSGEMGAVASINTFIPSGLRYVEGYKYKGVTNPIYKNLSAGMLWPKKYRVEVVNIPIDQATKIITATPNNNIKEKQVSDTISYGFGGSVSADGKKPGGSIEANLAYTKTTTYDQPDYETSQIKKTTKEAVWDTSFVETRDGYTPNSWNPVYGNQMFMRGRYSNVSPIDNIKKGGEVSSLISGGFSPKMGVVLASPNGTKKSQFVVRVSRMSDMYIMRWSGTEWGGENEINQNVPKEYNALMYEDVKFEIDWEQRTVRTI

SEQ ID NO: 20 - protein, 306 aa, synthetic construct
SFNEDNILGTTTKETDEDGKTATIITVKDRQIESYTSSDSATKENNTSLTVNANFLNDKYSSDLTTIMSLNGFIPSGKQYYFPPNNTWVGNMYWPSQYKTTVSSESLDQTVKIINTTPNNAIRNKDVSTSITYGVGGGVKVEGKTVGANIDANAAFTKTISYEQPDYETVQTQWTTDKTTWETNFTETTDGYTRTSWNTFYGNQIFMENPYTYNVKNNFTPNYKLSPLIAGGFSPSYGVVLKAPKNTKKSIVTVYMSRINDTYQQYWNGFNWVGRDFVDQSYSNWITNSNFKYEINWEEHTVKFIA

SEQ ID NO: 21 - protein, 306 aa, synthetic construct
SFNEDNILGTTTKETDEDGKTETIITVKDRQIESYTSSDSATKENNTSLTVNANFLNDKYSSDLTTIMSLNGFIPSGKQYYFPPNNTWVGNMYWPSQYKTTVSSESLDQTVKIINTTPNNAIRNKDVSTSITYGVGGGVKVEGKTVGANIDANAAFTKTISYEQPDYETVQTQWTTDKTTWETNFTETTDGYTRTSWNTFYGNQIFMENPYTYNVKNNFTPNYKLSPLIAGGFSPSYGVVLKAPKNTKKSIVTVYMSRINDTYQQYWNGFNWVGRDFVDQSYSNWITNSNFKYEINWEEHTVKFIA

SEQ ID NO: 22 - protein, 306 aa, synthetic construct
SFNEDNILGTTTKETDEDGKTETIITVEDRQIESYTSSDSATKENNTSLTVNANFLNDKYSSDLTTIMSLNGFIPSGKQYYFPPNNTWVGNMYWPSQYKTTVSSESLDQTVKIINTTPNNAIRNKDVSTSITYGVGGGVKVEGKTVGANIDANAAFTKTISYEQPDYETVQTQWTTDKTTWETNFTETTDGYTRTSWNTFYGNQIFMENPYTYNVKNNFTPNYKLSPLIAGGFSPSYGVVLKAPKNTKKSIVTVYMSRINDTYQQYWNGFNWVGRDFVDQSYSNWITNSNFKYEINWEEHTVKFIA

SEQ ID NO: 23 - protein, 305 aa, synthetic construct
SEDNIIGTTTQEIDEHGNVATIITVKNQQIESYTSTDSGTAKNRSTLTVNANFLNDKSYSNELTTILSLNGFIPSGRKFIFPKNNTLKGEMLWPQRYSTAVYNIPLDKSVKITNSTPDNTIRSKEVSNSITYGIGGGIKMEGKQPGANLDANAAITKTISYQQPDYETAKTTSTVTGVNWNTNFTETRDGYTRNSWNPVYGNQMFMYGRYTSNIRNNFTPDYQLSSLITSGFSPSYGLVLRAPKDVKKSRIKVVFARRSETYQQNWDGLNWWGRNFYDTKNPDSLSKVTLTFELDWQNHRVTFIE

SEQ ID NO: 24 - protein, 304 aa, synthetic construct
SEDNIIGTTTQEIDEHGNVETIITVKNQQIESYTSTDSGTAKNRSTLTVNANFLNDKYSNELTTILSLNGFIPSGRKFIFPKNNTLKGEMLWPQRYSTAVYNIPLDKSVKITNSTPDNTIRSKEVSNSITYGIGGGIKMEGKQPGANLDANAAITKTISYQQPDYETAKTTSTVTGVNWNTNFTETRDGYTRNSWNPVYGNQMFMYGRYTSNIRNNFTPDYQLSSLITSGFSPSYGLVLRAPKDVKKSRIKVVFARRSETYQQNWDGLNWWGRNFYDTKNPDSLSKVTLTFELDWQNHRVTFIE

SEQ ID NO: 25 - protein, 304 aa, synthetic construct
SEDNIIGTTTQEIDEHGNVETIITVENQQIESYTSTDSGTAKNRSTLTVNANFLNDKYSNELTTILSLNGFIPSGRKFIFPKNNTLKGEMLWPQRYSTAVYNIPLDKSVKITNSTPDNTIRSKEVSNSITYGIGGGIKMEGKQPGANLDANAAITKTISYQQPDYETAKTTSTVTGVNWNTNFTETRDGYTRNSWNPVYGNQMFMYGRYTSNIRNNFTPDYQLSSLITSGFSPSYGLVLRAPKDVKKSRIKVVFARRSETYQQNWDGLNWWGRNFYDTKNPDSLSKVTLTFELDWQNHRVTFIE

SEQ ID NO: 26 - protein, 22 aa, synthetic construct
MLKLKKIFLFTMISVSLCCGFG

SEQ ID NO: 27 - protein, 28 aa, synthetic construct
MKCMNLKKIFLFNTVIISILLVSSPVSA

SEQ ID NO: 28 - protein, 23 aa, synthetic construct
MKLKFALSVLSLSTILLSASPVL

SEQ ID NO: 29 - protein, 31 aa, synthetic construct
MIKLKKKNYKIWLSIAVLSAMLTTVPVFANF

SEQ ID NO: 30 - protein, 28 aa, synthetic construct
MVKLKKKNHKIWISIAVLSAMLTTVPVF

SEQ ID NO: 31 - protein, 28 aa, synthetic construct
MKIFRKSISKRIFFCAVLSGLMYTSPVQ

SEQ ID NO: 32 - protein, 13 aa, synthetic construct
AIISILLASSPVF

SEQ ID NO: 33 - protein, 24 aa, synthetic construct
MLKNSRMVLVLALLANFVVGTPVL

SEQ ID NO: 34 - protein, 15 aa, synthetic construct
GGGGSGGGGSGGGGS

SEQ ID NO: 35 - protein, 8 aa, synthetic construct
EFGSGSGS

SEQ ID NO: 36 - protein, 9 aa, synthetic construct
TYLPTNASL

SEQ ID NO: 37 - protein, 6 aa, synthetic construct
HHHHHH

SEQ ID NO: 38 - protein, 41 aa, synthetic construct
AATGGACTATCATATGCTTACCGTAACTTGAAAGTATTTCG

SEQ ID NO: 39 - protein, 36 aa, synthetic construct
ATGAATACTGCCATTTGTCTCAAGATCTAGTTACGC

SEQ ID NO: 40 - protein, 20 aa, synthetic construct
CAGTAAGTCAACTTCAATGT

SEQ ID NO: 41 - protein, 20 aa, synthetic construct
TCCCTCCTTACCCCATCTCA

SEQ ID NO: 42 - protein, 20 aa, synthetic construct
GGGTCCATCTCCTGCCCATC

SEQ ID NO: 43 - protein, 20 aa, synthetic construct
CTTTCAACAGTTTCCTGCGT

SEQ ID NO: 44 - protein, 318 aa, synthetic construct
MNSKKIITTILLSSMVIANLGLAQPVLANDLGSKSEIRKEENGNVTIITQNNKQIRKYSSTDSATTKSNSKITVDASFVDDKFSSEMTTIISLKGFIPSGRKIFALSKYRGVMRNPIKYMVDLKNNSLDSSVKIVDSVPKNTISTKEVNNTISYSIGGGIDTSNKASLNANYAVSKSISYVQPDYNTIQTNDTNSIASWNTEFAETRDGYNVNSWNIVYGNQMFMRSRYSGTSTTNFTPDYQLSSLITGGFSPNFGVVLTAPNGTKKSQIEISLKREINSYHIAWDTEWQGRNYPDSKIEETVKFELDWEKHTIRQIS

SEQ ID NO: 45 - protein, 322 aa, synthetic construct
MKRLKIISITLVLTSVISTSLPSTQTQVPASELNDINKIELKNLSGEIIKENGKEAIKYTSSDTASHKGWKATLSGTFIEDPHSDKKTALLNLEGFIPSDKQIFGSKYYGKMKNPKTYRINVKSADVNNNIKIANSIPKNTIDKKDVSNSIGYSIGGNISVEGKTAGAGINASYNVQNTISYEQPDFRTIQRKDDANLASWDIKFVETKDGYNIDSYHAIYGNQLFMKSRLYNNGDKNFTDDRDLSTLISGGFSPNMALALTAPKNAKESVIIVEYQRFDNDYILNWETTQWRGTNKLSSTSEYNEFMFKINWQDHKIEYYL

SEQ ID NO: 46 - protein, 319 aa, synthetic construct
MKTRIVSSVTTTLLLGSILMNPVAGAADSDINIKTGTTDIGSNTTVKTGDLVTYDKENGMHKKVFYSFIDDKNHNKKLLVIRTKGTIAGQYRVYSEEGANKSGLANPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 47 - DNA, 16 bp, synthetic construct
  source 1..16: mol_type = other DNA
aggaggaatttagatg

SEQ ID NO: 48 - DNA, 33 bp, synthetic construct
  source 1..33: mol_type = other DNA
tcacttccagagcagaaactcctctgcattttt

(19) United States
(12) Patent Application Publication
(10) Pub. No.: US 2025/0215058 A1
(43) Pub. Date: Jul. 3, 2025

(54) ENTEROCOCCUS PORE-FORMING TOXINS AND METHODS OF USE THEREOF

(71) Applicant: The Children's Medical Center Corporation, Boston, MA (US)

(72) Inventors: Min Dong, Weatogue, CT (US); Xiaozhe Xiong, Boston, MA (US); Songhai Tian, Brookline, MA (US)

(73) Assignee: The Children's Medical Center Corporation, Boston, MA (US)

(21) Appl. No.: 18/843,656

(22) PCT Filed: Mar. 3, 2023
(86) PCT No.: PCT/US2023/063639

§ 371 Date: Sep. 3, 2024

Related U.S. Application Data
(60) Provisional application No. 63/316,921, filed on Mar. 4, 2022.

(51) Int. Cl. C07K 14/315; A61K 38/00

(52) U.S. Cl. CPC C07K 14/315; A61K 38/00

(57) ABSTRACT

The present application describes in part isolated and modified Enterococci toxin (Epx) polypeptides, immunogenic compositions comprising Exp polypeptides, nanopores formed by Epx polypeptides, apparatus comprising Epx nanopores, and methods of use thereof.

### RELATED APPLICATION

This application claims the benefit under 35 U.S.C. § 119 (e) to U.S. Provisional Application No. 63/316,921, filed Mar. 4, 2022, and entitled “ENTEROCOCCUS PORE-FORMING TOXINS AND METHODS OF USE THEREOF.” the entire contents of which are incorporated herein by reference.

### FEDERALLY SPONSORED RESEARCH

This invention was made with government support under Grant Number NS080833, awarded by the National Institutes of Health. The Government has certain rights in the invention.

### BACKGROUND

One aspect of bacterial pathogenicity is the production of toxins that can damage or kill host cells. Despite being toxic, some of these toxins have proven useful in therapeutic and biotechnology settings. For example, Botulinum toxins have been used to treat diseases associated with unwanted neuronal activity and have been used in cosmetic applications. Thus, study of bacterial toxins may lead to discovery of useful toxins, development of toxin variants, and use of toxins in therapeutics and biotechnology.

### SUMMARY

In some aspects, the instant application discloses uncharacterized small β-barrel pore forming toxins (PFTs) in E. faecalis, E. faecium, and E. hirae. Structural studies revealed that these toxins form a sub-class of the haemolysin family. Through a genome-wide CRISPR-Cas9 screen, the HLA-I complex was identified as a receptor for two of these toxins (Epx2 and Exp3), which recognize human HLA-I and homologous MHC-I of equine, bovine, and porcine, but not murine origin. In some embodiments, it was demonstrated that a toxin-harboring E. faecium strain induces death of peripheral blood mononuclear cells (PBMCs) and damages intestinal organoids in a toxin-dependent manner during co-culture, demonstrating toxin-mediated virulence.

In some aspects, the present application discloses, an isolated Enterococci toxin (Epx) polypeptide, comprising an amino acid sequence that is at least 85% identical to any one of SEQ ID NOs: 1-8, wherein the amino acid sequence is not 100% identical to any one of SEQ ID NOs: 1-8. In some embodiments, the isolated Epx polypeptide comprises an amino acid sequence that is at least 95% identical to any one of SEQ ID NOs: 1-8. In some embodiments, the isolated Epx polypeptide further comprises a signal sequence. In some embodiments, the signal sequence is selected from the group consisting of SEQ ID NOs: 26-33.

In some aspects, the present application discloses, a nanopore comprising the isolated Epx polypeptide as described herein. In some aspects, the present application discloses an apparatus comprising a nanopore, as described herein, and a membrane. In some embodiments, the nanopore is disposed in the membrane.

In some aspects, the present application discloses a modified Epx polypeptide, comprising an amino acid sequence that is at least 85% identical to any one of SEQ ID NOs: 9-16 and an amino acid substitution at a position corresponding to K50 and/or K56 of SEQ ID NO: 9. In some embodiments, the modified Epx polypeptide comprises an amino acid sequence that is at least 95% identical to any one of SEQ ID NOs: 9-16 and an amino acid substitution at a position corresponding to K50 and/or K56 of SEQ ID NO: 9. In some embodiments, the modified Epx polypeptide comprises the amino acid sequence of any one of SEQ ID NOs: 9-16 and an amino acid substitution at a position corresponding to K50 and/or K56 of SEQ ID NO: 9. In some embodiments, the amino substitution introduces a neutral amino acid or a negatively charged amino acid. In some embodiments, the amino acid substitution corresponds to K50E or K50A of SEQ ID NO: 9. In some embodiments, the amino acid substitution corresponds to K54E or K54A of SEQ ID NO: 9. In some embodiments, the modified Epx polypeptide comprises an amino acid substitution corresponding to K50E or K50A of SEQ ID NO: 9, and an amino acid substitution corresponding to K56E or K56A of SEQ ID NO: 9. In some embodiments, the modified Epx polypeptide comprises an amino acid sequence that is at least 85% identical to of any one of SEQ ID NOs: 17-25. In some embodiments, the modified Epx polypeptide comprises an amino acid sequence that is at least 95% identical to of any one of SEQ ID NOs: 17-25. In some embodiments, the modified Epx polypeptide comprises the amino acid sequence of any one of SEQ ID NOs: 17-25.

In some aspects, the present application discloses a composition comprising the modified Exp polypeptide as described herein, or a fragment thereof. In some embodiments, the composition further comprises an antigen. In some embodiments, the antigen is a viral antigen, a bacterial antigen, a cancer antigen, a fungal antigen, or a parasitic antigen. In some embodiments, the antigen in a peptide antigen. In some embodiments, the antigen is conjugated to the Exp polypeptide. In some embodiments, the antigen is a peptide antigen fused to the Exp polypeptide, forming a fusion protein. In some embodiments, the fusion protein from N-terminal to C-terminal comprises the peptide antigen then the modified Epx polypeptide or a fragment thereof. In some embodiments, the fusion protein from N-terminal to C-terminal comprises the n-terminal of the modified Epx polypeptide or a fragment thereof then the peptide antigen.

In some embodiments, the composition is an immunogenic composition. In some embodiments, the immunogenic composition is a vaccine. In some embodiments, the modified Exp polypeptide is used as an adjuvant.

In some aspects, the present application discloses a method of inducing an immune response against Enterococci in a subject, the method comprising administering to the subject a modified Exp polypeptide described herein or the composition described herein. In some embodiments, the Enterococci is a multi-drug resistant Enterococci. In some aspects, the present application discloses a method of inducing an immune response against an antigen in a subject, the method comprising administering to the subject the composition disclosed herein. In some embodiments, the method is therapeutic. In some embodiments, the method is prophylactic. In some embodiments, the subject is a mammalian subject. In some embodiments, the subject is a human subject.

In some aspects, the present application discloses a method of blocking MHC class I activity, the method comprising contacting an MHC class I receptor with the isolated Epx polypeptide described herein, the modified Epx polypeptide described herein, or the composition described herein. In some embodiments, the contacting occurs in an cell free assay. In some embodiments, the contacting occurs in in vitro cell culture. In some embodiments, the contacting occurs in a subject. In some embodiments, the subject is an animal. In some embodiments, the subject is a human. In some embodiments, the Epx polypeptide binds to a α1-α2 region of the MHC class I α-subunit.

In some aspects, the present application discloses a method of treating a disease associated with detrimental MHC class I activity, the method comprising administering to a subject the isolated Epx polypeptide described herein, the modified Epx polypeptide described herein, or the composition described herein. In some embodiments, the disease is selected from the group consisting of cancer, an autoimmune disease, a bacterial infection, a viral infection, a parasitic infection, or a fungal infection. In some embodiments, the disease is selected from the group consisting of idiopathic inflammatory muscle diseases, diabetes, chronic inflammation, rheumatoid Arthritis, ankylosing spondylitis, asthma, Alzheimer's disease, Inflammatory bowel disease, obesity, Fatty liver disease and Endometriosis. In some embodiments, the subject is a mammalian subject. In some embodiments, the subject is a human subject.

In some aspects, the present application discloses a nucleic acid sequence encoding the isolated Epx polypeptide described herein, the modified Epx polypeptide described herein, or the fusion protein described herein. In some embodiments, the present application discloses a vector comprising the nucleic acid sequence. In some embodiments, the vector is a plasmid.

In some aspects, the present application discloses a cell comprising the isolated Epx polypeptide described herein, the modified Epx polypeptide described herein, or the fusion protein described herein, the nucleic acid sequence described herein, or the vector described herein. In some embodiments, the cell is a bacterial cell. In some embodiments, the cell is an Enterococci cell. In some embodiments, the cell is a mammalian cell.

In some aspects, the present application discloses a method of producing an Exp polypeptide, the method comprising culturing the cell described herein under conditions that permit expression of the Exp polypeptide. In some embodiments, the method further comprises isolating the Exp polypeptide.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

FIGS. 1A-1J shows identification and analysis of Enterococcus pore-forming toxins (Epxs). FIG. 1A shows the maximum likelihood phylogeny of Epx toxins based on amino acid alignments. The scale bar represents the mean number of amino acid substitutions per site. Each toxin was assigned a color code. NetB: C. perfringens necrotic enteritis B-like toxin; Hla: S. aureus α-hemolysin; Delta: C. perfringens delta toxin. FIG. 1B shows the 16S based phylogeny of the Enterococcus genus showing that Epxs are detected in three distinct species. FIG. 1C shows the global geographical distribution and source of isolation for Epx-carrying Enterococcus strains (marked with colored dots). FIGS. 1D-1F show the genome-based, phylogenetic trees comparing Epx-carrying Enterococcus isolates with the diverse sets of publicly available (RefSeq NCBI dataset) E. faecalis (n=1743) (FIG. 1D), E. faecium (n=2197) (FIG. 1E), and E. hirae (n=190) (FIG. 1F) data. Branch lengths reflect the number of substitutions per site. FIG. 1G shows comparison of Epx2-carrying repUS15 plasmids found in DIV0147 and 58 M. TIVSS. Type IV secretion system and pilus assembly; Urease, urea catabolism operon. FIG. 1H shows Hela cells were exposed to Epx1, 2, 3, and 4 for 4 h. Cell viability was measured using MTT assays. Error bars indicate mean±SD, N=3. FIG. 1I shows Epx2 (2 μM) induced leakage of liposomes and release of fluorescent dye Sulforhodamine B. Representative curves are shown from two independent experiments. FIG. 1J shows the negative staining EM of liposomes after incubation with Epx2 (2 μM), showing that Epx2 forms pores on liposomes. Scale bar, 50 nm. Sec also FIGS. 8A-10K and Tables 2-3.

FIGS. 2A-2F show the crystal structure of Epx4 octameric pore. FIG. 2A shows the bottom and side view of a ribbon diagram of the 3.0 Å crystal structure of the Epx4 pore. MPD molecules bound to Epx4 are shown as spheres. FIG. 2B shows the structure of the Epx4 protomer. The N-terminus (N), C-terminus (C), and the residue numbers for the top domain and the stem domain are marked. FIG. 2C shows the ribbon diagram of the rim domain of Epx4. The side chains of exposed aromatic residues are shown as sticks and labeled. FIG. 2D shows the superimposition of the Epx4 and Hla (PDB: 7AHL) protomers (Song et al., 1996). The N-terminal top region and the stem domain are enlarged in the right panels. FIG. 2E shows the comparison of the size and configuration of the Epx4 pore versus the Hla pore (PDB: 7AHL). FIG. 2F shows the electrostatic surface representation of the Epx4 pore. Viewed from the membrane side (left) and the pore interior side (right). Electrostatic potential is expressed as a spectrum ranging from −10 KT/c to +10 kT/e. TM, transmembrane. See also Table 4.

FIGS. 3A-3F show the cryo-EM structure of Epx1 prepore. FIG. 3A shows the 2D class averages of Epx1 particles. FIG. 3B shows the Cryo-EM map (gray, 2.9 Å resolution) superimposed onto the atomic model of the octameric Epx1 prepore. Density was not observed for residues 176 to 184, and these are shown as dashed lines. FIG. 3C shows the structure of Epx1 protomer. The regions that were not resolved are shown as dashed lines. FIG. 3D shows the structure of the Epx1 protomer and its superimposition with the Epx4 protomer. FIG. 3E shows the top domains of Epx1 (left panel) and Epx4 (right panel) are enlarged to indicate crucial polar interactions (dashed lines). FIG. 3F shows the mutations that disrupt polar interactions in the top domains of Epx1 (K50E, K50E/K54E), Epx4 (K51E, K51E/K57E), and Epx2 (K50E, K50E/K56E) abolished cytotoxicity on HeLa cells. Error bars indicate mean±SD, N=3. See also FIGS. 9-12E and Table 4.

FIGS. 4A-4K show the genome-wide CRISPR/Cas9 screen identifies MHC/HLA-I as a receptor for Epx2. FIG. 4A shows the schematic diagram of the screening process. NGS, next-generation sequencing. FIG. 4B show the genes identified from the screen were plotted based on the number of sgRNAs (y-axis) and total sgRNA reads (x-axis). FIG. 4C shows the schematic drawing of the HLA-I complex. FIG. 4D shows the immunoblot analysis of cell lysates showed that B2M expression was abolished in mixed stable B2M knockout (KO) Hela cells. Actin served as a loading control. FIGS. 4E-4F show the mixed stable B2M KO and HLA-A KO HeLa cells showed reduced sensitivity to Epx2 compared with wild-type (WT) HeLa cells (FIG. 4E). The IC₅₀ values were plotted in FIG. 4F. Error bars indicate mean±SD, N=3, **, p<0.01. FIG. 4G shows the cell lysates of four human cell lines (U937, HeLa, U2OS, Daudi) that were analyzed by immunoblot to detect their endogenous B2M levels. Actin served as the loading control. FIGS. 4H-4I show the sensitivity of U937, HeLa, U2OS, and Daudi cells to Epx2 was analyzed using MTT assays (FIG. 4H). The IC₅₀ values were plotted in panel (FIG. 4I). Error bars indicate mean±SD, N=3. FIG. 4J shows the immunostaining analysis showed that GST-tagged Epx2 and Epx3 bound to WT HeLa cells, but not B2M KO cells. GST-Epx2 and -Epx3 were detected using anti-GST antibody. Nuclei were labeled with DAPI. Scale bar, 5 μm. FIG. 4K shows immunoblot analysis showed that GST tagged Epx2 and Epx3, but not Epx4, pulled down endogenous HLA-A and B2M from HeLa cell lysates. (FIGS. 4D, 4G, 4J, 4K): representative images were from one of three independent experiments. See also FIGS. 13A-13H.

FIGS. 5A-5G show Epx2 and 3 recognize HLA/MHC-I complexes. FIG. 5A shows the schematic diagram of B2M, B2M fused with a peptide (pep-B2M), HLA-A, B2M fused with HLA-A, and a fusion protein containing a peptide, B2M, and HLA-A. All are tagged with 3× FLAG at their C-termini. FIG. 5B shows HEK293 cells were transfected with the above constructs, and cell lysates were subjected to pull-down assays using GST-tagged Epx2, Epx3, and Epx4. B2M-HLA fusion proteins (B2M-HLA and pep-B2M-HLA) but not B2M alone (B2M and pep-B2M) or HLA-A alone can be pulled down by GST-Epx2 and -Epx3. FIG. 5C shows FLAG-tagged HLA-A, HLA-B, HLA-C, and their fusion proteins with B2M or B2M plus a peptide (pep-B2M) were expressed in HEK293 cells. Pull-down experiments with GST-Epx2 were carried out. FIG. 5D shows direct binding of GST-Epx2 to biotin-labeled HLA-I complex was characterized using biolayer interferometry. Neonatal Fc receptor (FcRn) and empty probe were analyzed as controls. FIG. 5E shows FLAG-tagged human, murine, equine, bovine, and porcine B2M-HLA fusion proteins were expressed in HEK cells. Cell lysates were collected and split into two equal parts for pull-down assays using GST-tagged Epx2 and Epx3, respectively. FIGS. 5F-5G show six chimeric FLAG-tagged MHC-I complexes were generated by switching α1-α2, α3, or B2M between human (*) and murine (\hat{ }) versions (FIG. 5F). These chimeric MHC-I complexes were expressed in HEK cells, and pull-down assays were carried out with GST-Epx2 (FIG. 5G). FIGS. 5B, 5C, 5E, 5G: representative images were from one of three independent experiments. See also FIGS. 14A-14M.

FIGS. 6A-6M show IFN-γ sensitizes human cells and intestinal organoids to Epx2 and Epx3. FIG. 6A shows IFN-γ treatment elevated MHC-I complex levels in human and mouse cell lines, as demonstrated by immunoblot analysis of B2M in cell lysates. Actin served as a loading control. FIGS. 6B-6D show IFN-γ treatment increased sensitivity of human cell lines (HeLa, U2OS and Huh7) to Epx2 (FIG. 6B), but did not change the sensitivity of mouse cell lines (BMDM, CT26 and Raw) (FIG. 6C). IC₅₀ values for Epx2, Epx3, and Epx4 are summarized in (FIG. 6D). Error bars indicate mean±SD, N=3. *, p<0.05; **, p<0.01 (Student's t-test). (FIG. 6E) Immunoblot analysis showed that GST-Epx2 did not pull down mouse B2M from BMDM lysates, whereas GST-Epx3 weakly pulled down B2M from BMDM lysates upon treatment with IFN-γ. FIG. 6F shows IFN-γ treatment increased MHC-I levels in primary cultured mouse endothelial cells (mEC) and human umbilical vein endothelial cells (HUVEC). Actin served as a loading control. FIGS. 6G-6H show IFN-» treatment increased sensitivity of HUVEC, but not mEC, to Epx2 (FIG. 6G). IC₅₀ values for Epx2, Exp3, and Epx4 on HUVEC and mEC with and without IFN-γ treatment are summarized in (FIG. 6H). Error bars indicate mean±SD, N=3. **, p<0.01 (Student's t-test). FIG. 6I shows IFN-γ treatment increased B2M levels in cultured human intestinal organoids. FIG. 6J shows representative images showing that Epx2 (100 ng/mL, 30 minutes incubation) induced death of cultured human intestinal organoids. Scale bar, 100 μm. FIGS. 6K-6M show human intestinal organoids are sensitive to both Epx2 (FIG. 6K) and Epx3 (FIG. 6L), and their sensitivities are further increased after IFN-γ treatment. These organoids are not sensitive to Epx4 and IFN-γ treatment did not alter the sensitivity to Epx4 (FIG. 6M). Ctrl, control. *, p<0.05; **, p<0.01 (Student's t-test). (FIGS. 6A, 6E, 6F, 6I, 6J): representative images were from one of two independent experiments. See also FIG. 14.

FIGS. 7A-7J show the co-culture with E. faecium DIV0147 damages human PBMCs and intestinal organoids through Epx2. FIG. 7A shows a rabbit polyclonal antibody against Epx2 neutralized the toxicity of Epx2 toxin on HeLa cells. IgG served as a control. FIG. 7B shows E. faecium DIV0147 culture supernatant induced death of Hela cells, and this toxicity was neutralized by an Epx2 antibody. The supernatant from a control strain that lacks epx2 gene, E. faecium DIV0391, did not show any toxicity to cells. Scale bar: 50 μm. FIGS. 7C-7D show the co-culture with E. faecium DIV0147 (MOI=800, 6 hours) resulted in death of U937 (FIG. 7C) and HeLa cells (FIG. 7D). This toxicity is neutralized by an Epx2 antibody. Co-culture with a control strain E. faecium DIV0391 did not show any toxicity to cells. FIG. 7E shows the co-culture with E. faecium DIV0147 did not affect the viability of B2M KO HeLa cells. FIG. 7F shows the co-culture with E. faecium DIV0147 (MOI=800, 4 hours) damaged human PBMCs measured by LDH release assays. This toxicity was neutralized by an Epx2 antibody. Co-culture with a control strain E. faecium DIV0391 did not show any toxicity to cells. FIGS. 7G-7J shows human intestinal organoids were cultured as monolayers on trans-wells. E. faecium DIV0147 was added to the upper compartment during co-culture (MOI=800, 8 hours), which damaged intestinal organoids (detected by LDH release assays) and increased the dye leakage into the lower chamber (FIGS. 7H-7J). This toxicity is neutralized by an Epx2 antibody. C-G: **, p<0.01, NS p>0.05 (Student's t-test). See also FIGS. 14A-14M.

FIGS. 8A-8F show analysis of the epx loci, related to FIGS. 1A-1J. The epx-carrying contigs were retrieved from publicly available draft genomes. When applicable, putative ORFs function (or homologous proteins), promoters and transcriptional terminators (predicted only for adjacent regions of epx) were indicated. For epx1, the specific sequence of the upstream promoter and downstream terminator (the computed AGs free energy value of the stem-loop structure is indicated) are shown. For complete genomes only, the chromosomal or plasmidic location of the epx locus is indicated. For genomes with high quality, annotated assemblies available in public databases, contigs carrying the epx genes were aligned using the progressiveMAUVE (1) plugin in Geneious Prime 2021.2.2. The alignments of locally collinear blocks were further enriched using image editing software for clarity and complementary information. For regions upstream and downstream of the epx genes, bacterial promoters and transcriptional terminators were predicted in silico using BPROM and ARNold (Naville et al., 2011). In silico analysis predicted that, in most cases, the toxin gene would be individually transcribed. A notable exception was epx1, which was predicted to be a part of a three-gene operon including two upstream genes coding for a protein of unknown function and a putative phospholipase with ˜50% amino acid identity to homologous proteins found in Clostridium botulinum and Chlamydia trachomatis. Efs, E. faecalis; Efm, E. faecium; Ehi, E. hirae; RM, restriction-modification; TA, toxin-antitoxin; fam., family; TCS, two component system; deH, dehydrogenase. In FIG. 8A, sequences correspond to SEQ ID NOs: 47 (AGGAGGAATTTAGATG) and 48 (TCACTTCCAGAGCAGAAACTCCTCTGCATTTTT). FIGS. 8A-8G correspond to analysis for Exp polypeptides Exp1-Exp8, respectively.

FIG. 9 shows the sequence alignment of Epxs and Hla family toxins, related to FIG. 1. Structure-based sequence alignment of Epxs and delta toxin (Uniprot No.: B8QGZ7), Hla (Uniprot No.: P09616), and NetB (Uniprot No.: A8ULG6). Residue numbers are shown as dots every ten residues above the alignment. Conserved residues are colored in light gray; identical residues are shaded. β labels indicate the β-strands. The alignment was done using ESPript server (espript.ibcp.fr/ESPript/ESPript). Sequences shown correspond to SEQ ID NOs: 12 (Epx4), 10 (Epx2), 16 (Epx8), 15 (Epx7), 14 (Epx6), 13 (Epx5), 11 (Epx3), 9 (Epx1), 44 (delta), 45 (NetB), 46 (Hla).

FIGS. 10A-10K shows that Epxs are cytotoxic to mammalian cells and Epxs form oligomeric pores on liposomes, related to FIG. 1 and FIG. 3. FIG. 10A shows the schematic diagram of Epx1, 2, 3, and 4 proteins. The numbers indicate the position of amino acid residues. The N-terminal signal sequences are predicted using SMART server (smart.embl-heidelberg.de). FIG. 10B shows recombinant Epx1, 2, 3, and 4 proteins without signal sequences were expressed and purified from E. coli and are shown on SDS-PAGE gels with Coomassie blue staining. FIG. 10C shows recombinantly produced Epx1, 2, 3, and 4 proteins (100 μg/mL, 10 min incubation) induced morphological changes in HEK293 cells, while heat-denatured proteins showed no toxicity. Scale bar: 50 μm. FIG. 10D shows a panel of human cell lines (HEK293, A549, Huh7, U2OS, and 5637) were exposed to serial two-fold dilutions of Epx1, Epx2, Epx3, or Epx4 for 4 h. Cell viability was measured using MTT assays and plotted over toxin concentrations. Error bars indicate mean±SD, N=3. FIG. 10E shows Vero cells from African green monkeys, immortalized bone marrow-derived macrophage (BMDM) cells from mice, MDCK cells from dogs, and S2R+ cells from drosophila were exposed to serial dilutions of Epx1, Epx2, Epx3, or Epx4 for 4 h. Cell viability was measured using MTT assays and plotted over toxin concentrations. Error bars indicate mean±SD, N=3. FIG. 10F shows Epx1, Epx3, and Epx4 (10 μM) induce leakage of liposomes and release of fluorescent dye Sulforhodamine B. The amounts of leakage were plotted as percentages of the total amount of Sulforhodamine B released by the detergent Triton X-100 (4% v/v). FIG. 10G shows Epxs with or without incubation with liposomes (1 h) were analyzed on 4%-20% SDS-PAGE and by Coomassie blue staining. Epx1 forms high molecular-weight SDS-resistant oligomers even without incubation with liposomes, which was abolished after heating. Epx2, Epx3, and Epx4 form high molecular-weight SDS-resistant oligomers after incubation with liposomes. FIGS. 10H-10K show electrical properties of the Epx1 pore on planar lipid bilayers. Under +20 mV holding potential, addition of Epx1 at 100 μg/mL into the planar lipid bilayers resulted in multiple pores (FIG. 10H). Representative trace of Epx1 pores at +50 mV potential shown in FIG. 10I. Current histogram of Epx1 pore shown in (FIG. 10J). The data were fitted by a single Gaussian distribution to determine the mean current (black trace). I/V plot of the Epx1 pore shown in FIG. 10K.

FIGS. 11A-11E Cryo-EM data collection and processing, related to FIG. 3. FIG. 11A shows representative Cryo-EM micrograph of Epx1. Scale bar=100 nm. FIG. 11B shows the corresponding Fourier transformation of FIG. 11A. FIG. 11C shows 1,963,299 particles were picked from 4,920 micrographs. Through 2D classification and three rounds of 3D classification, 119,503 octamer particles were selected for the final reconstruction. FIG. 11D shows the gold-standard FSC curve based on the FSC-0.143 criterion. FIG. 11E shows local resolution maps of Epx1 oligomers shown as a spectrum ranging from 2.7 Å to 3.5 Å.

FIGS. 12A-12E show structural characterization of Epx1 and the top domain, related to FIG. 3. FIG. 12A shows the cartoon representation of the rim domain of Epx1. The side chains of exposed aromatic residues are shown in stick representation and labeled. FIG. 12B shows electrostatic surface views of the Epx1 pore viewed from the external surface (left panel) or as an internal slide (right panel). Electrostatic potential is expressed as a spectrum ranging from −10 kT/c to +10 kT/c. TM, transmembrane. FIG. 12C shows the comparison of Epx1 oligomers with the reported prepore structure of γ-hemolysin (PDB: 4P1Y) (Yamashita et al., 2014). The two components of γ-hemolysin, LukF and HIg2, are shown. FIG. 12D shows the circular dichroism spectroscopy analysis of Epxs and Epx mutants. FIG. 12E shows the designed mutations in the top domains of Epx1 (K50E, K50E/K54E), Epx2 (K50E, K50E/K56E), and Epx4 (K51E, K51E/K57E) reduced the efficacy of forming uniform SDS-resistant oligomers, analyzed by 4%-20% SDS-PAGE gels and Coomassie blue staining.

FIGS. 13A-13I show genome-wide CRISPR-Cas9 screen and validation, related to FIG. 4. FIG. 13A shows the recovery rates of genes identified in initial cell library (R0) compared with the original GeCKO-v2 library. FIG. 13B shows genes identified in round 2 (R2, the final surviving cells) are plotted based on their fold-enrichment of total sgRNA reads compared with the initial cell library. Selected top hits are marked. FIG. 13C shows mixed stable GAGE1 and SNX17 KO HeLa cells were generated using CRISPR-Cas9 approach. These KO cells and the control WT cells were exposed to serial dilutions of Epx2 for 4 h, and cell viability was measured using MTT assays. FIGS. 13D-13F show WT HeLa and B2M KO cells were exposed to serial dilutions of Epx1 (FIG. 13D), Epx3 (FIG. 13E), and Epx4 (FIG. 13F) for 4 h. Cell viability was measured using MTT assays. FIG. 13G-13H shows the sensitivity of U937, HeLa, U2OS, and Daudi cells to Epx3 and Epx 4, respectively, as analyzed by MTT assays. FIG. 13I shows a western blot of for GST after GST-Epx2 was incubated with HeLa and B2M KO cells on ice (50 μg/mL, 40 minutes). Cell lysates were harvested and analyzed by immunoblot detecting bound GST-Epx2 using an anti-GST antibody. Actin served as a loading control. (FIGS. 13C-13G): Error bars indicate mean±SD, N=3.

FIGS. 14A-14M shows IFN-γ treatment sensitizes cells to Epx3 and characterization of an Epx2 antibody, related to FIGS. 5-7. FIG. 14A shows GST-Epx2 and recombinantly purified B2M-Fc were mixed and incubated for 2 h. Pull-down assays were carried out using glutathione agarose beads and samples were run on 12% SDS-PAGE gels with Coomassie blue staining. FIGS. 14B-14C show the binding kinetics and affinity between GST-Epx2 and HLA-I complex were determined using biolayer interferometry. Representative sensorgrams of different concentrations of GST-Epx2 are shown in FIG. 14B and binding affinities are listed in FIG. 14C. FIGS. 14D-14E show the sensitivities of three human cell lines (FIG. 14A) and three mouse cell lines (FIG. 14B) to Epx3, with or without IFN-γ treatment, were analyzed by MTT assays. FIGS. 14F-14G show human (FIG. 14C) and mouse (FIG. 14D) cells are insensitive to Epx4, and sensitivity was not changed by IFN-γ treatment. FIG. 14H shows HUVEC became highly sensitive to Epx3 after exposure to IFN-γ. IFN-γ treatment also slightly increased the sensitivity of mEC to Epx3. FIG. 14I shows HUVEC and mEC were not sensitive to Epx4 and IFN-γ treatment did not change their sensitivity to Epx4. FIG. 14J shows human organoids were not insensitive to Epx4 and IFN-γ treatment did not change their sensitivity to Epx4. FIG. 14K shows dot-blot analysis showing that the rabbit polyclonal Epx2 antibody recognizes Epx2 but not Epx1, 3, or 4. FIG. 14L shows Epx2 antibody does not affect the toxicity of Epx3 on HeLa cells. FIG. 14M shows culture supernatant of E. faecium DIV0147 was subjected to mass spectrometry analysis, which identified eight peptide fragments in Epx2. The protein sequence of Epx2 (SEQ ID NO: 10) is shown, with identified eight peptides underlined. All panels: error bars indicate mean±SD, N=3.

FIGS. 15A-15C show mutations that disrupt polar interactions in the top domains of Epx1 (K50A, K50E, K50E/K54E), Epx4 (K51A, K51E, K51E/K57E), and Epx2 (K50A, K50E, K50E/K56E) reduced or abolished cytotoxicity on HeLa cells. Error bars indicate mean±SD, N=3.

FIG. 16 shows the NCBI reference numbers, strains, sample collection type and sample collection location of the Epx polypeptides.

### DETAILED DESCRIPTION

Conversion of harmless bacteria into pathogens by mobile elements has been described since the landmark 1950s finding that diphtheria toxin was conveyed by a temperate phage (Freeman, 1951). Enterococci are a part of gut commensal bacteria of land-based animals (Lebreton et al., 2017). Since the 1970's, Enterococcus faecalis and E. faecium have emerged as leading causes of multidrug resistant (MDR) infections (Arias and Murray, 2012; Fiore et al., 2019; Gilmore et al., 2013; Lebreton et al., 2013; Van Tyne and Gilmore, 2014). Recent studies have also reported a role of E. faecalis in alcoholic liver disease (Duan et al., 2019), and a role of E. hirae, the third most abundant Enterococcus species in human microbiota, in regulating immune responses to tumor antigens (Fluckiger et al., 2020). Enterococci are well known for their intrinsic and recently acquired resistance to antibiotics (Van Tyne and Gilmore, 2014), leading to high mortality in infections that are difficult to eradicate. Some isolates of E. faecalis express a post-translationally modified anti-microbial peptide bacteriocin known as cytolysin, which can lyse both bacteria and eukaryotic cells and contribute to pathogenesis (Coburn et al., 2004; Van Tyne et al., 2013). However, the genus Enterococcus is not known to express any potent protein toxin family with an established specificity targeting human and animal cells.

Pore-forming toxins (PFTs) are the most common class of bacterial toxins (Dal Peraro and van der Goot, 2016; Los et al., 2013). They are produced as soluble monomers that oligomerize and form transmembrane pores on cell surfaces. A variety of PFTs have evolved to disrupt epithelial barriers, disable immune cells, and damage tissues. PFTs can be divided into α-PFTs with transmembrane pores composed of α-helices, and β-PFTs with pores composed of β-barrels. β-PFTs further include two classes of small β-barrel PFTs, the haemolysin and aerolysin families, as well as the cholesterol-dependent cytolysin family that forms large pores (Dal Peraro and van der Goot, 2016).

The well-studied Staphylococcus aureus α-hemolysin (Hla, also known as αHL or α-toxin) is the archetype for the haemolysin family (Berube and Bubeck Wardenburg, 2013). It is produced as a 292-residue monomer and assembles into a mushroom-shaped heptameric pore (Song et al., 1996). Other haemolysin family members include S. aureus leucocidin toxins, necrotic enteritis B-like toxin (NetB), beta toxin and delta toxin from C. perfringens, and Vibrio cholerae cytolysin toxin (VCC) (Dal Peraro and van der Goot, 2016). Crystal structures of these toxins show highly conserved conformations in their monomeric states and assembled pores, with a variation consisting of bi-component leucocidin toxins forming hetero-octameric pores composed of four units of each component in alternating order (De and Olson, 2011; Guillet et al., 2004; Huyet et al., 2013; Olson et al., 1999; Pedelacq et al., 1999; Savva et al., 2013; Song et al., 1996; Sugawara et al., 2015; Tanaka et al., 2011; Yamashita et al., 2014; Yamashita et al., 2011; Yan et al., 2013). The archetypical aerolysin forms heptameric β-barrel transmembrane pores as well but differs from Hla in the overall domain arrangement (Degiacomi et al., 2013; Parker et al., 1994).

Although small β-barrel PFTs can form pores non-specifically at high concentrations in vitro, specific host protein receptors have been identified, establishing the key role of receptors in determining toxin host species and cell type selectivity (Alonzo et al., 2013; Bruggisser et al., 2020; DuMont et al., 2013; Reyes-Robles et al., 2013; Spaan et al., 2013; Spaan et al., 2017; Tromp et al., 2018; Wilke and Bubeck Wardenburg, 2010). For instance, S. aureus leucocidin toxins PVL, HlgCB, and LukAB recognize the human orthologs of their respective receptors, but not the murine orthologs (DuMont et al., 2013; Perelman et al., 2021; Spaan et al., 2013; Spaan et al., 2017).

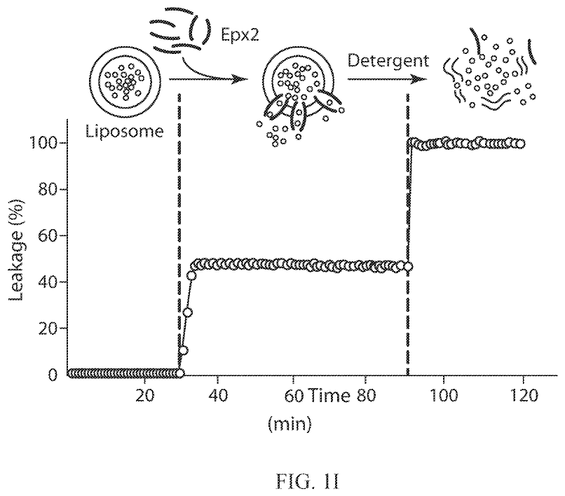

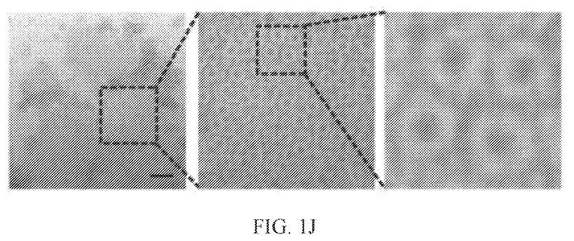

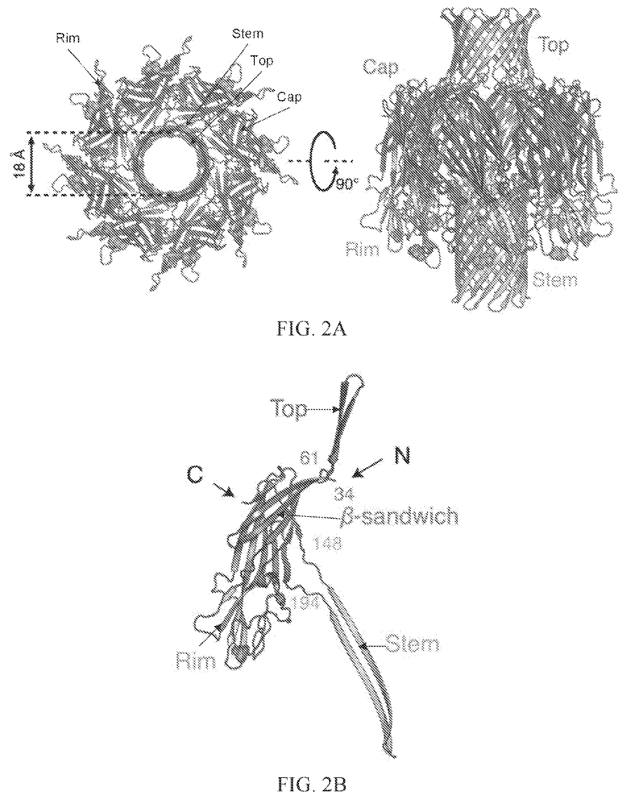

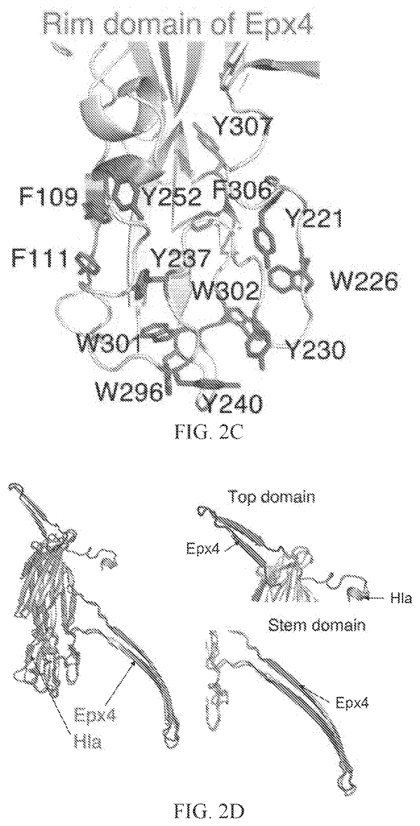

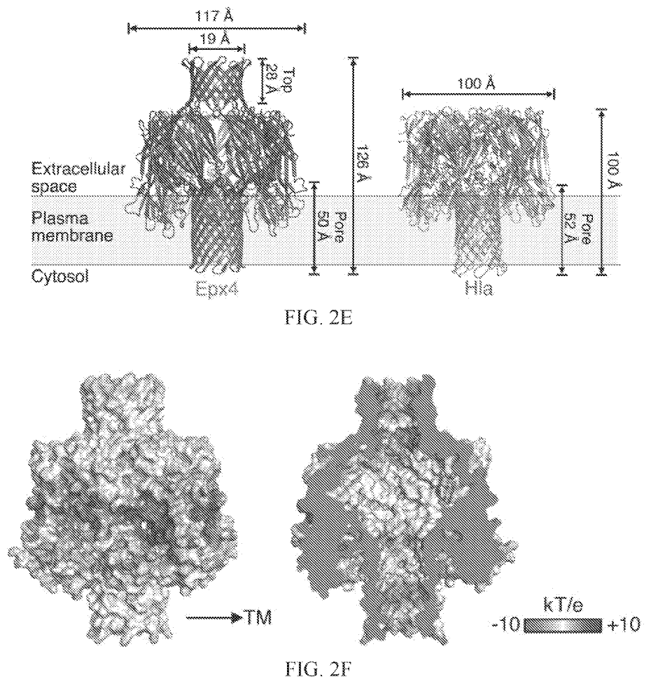

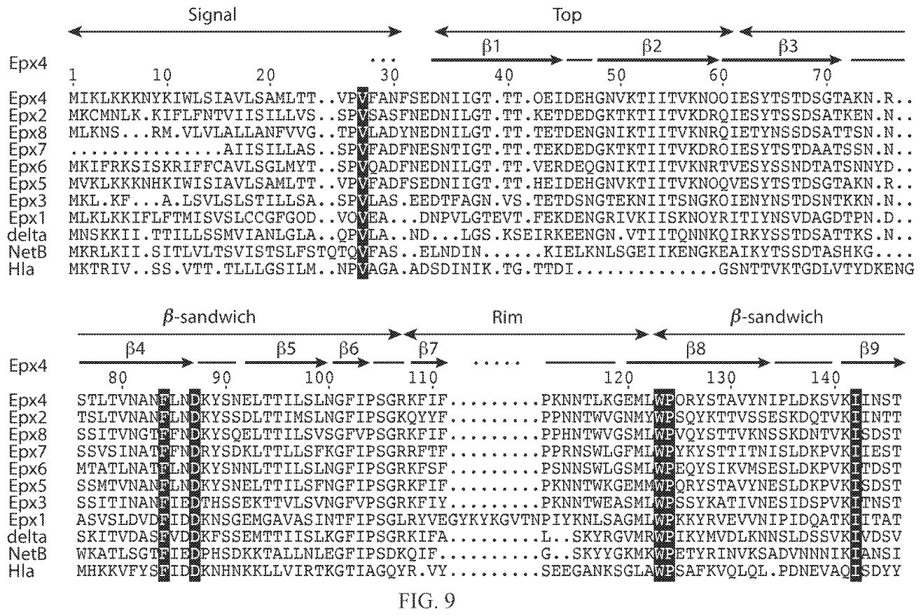

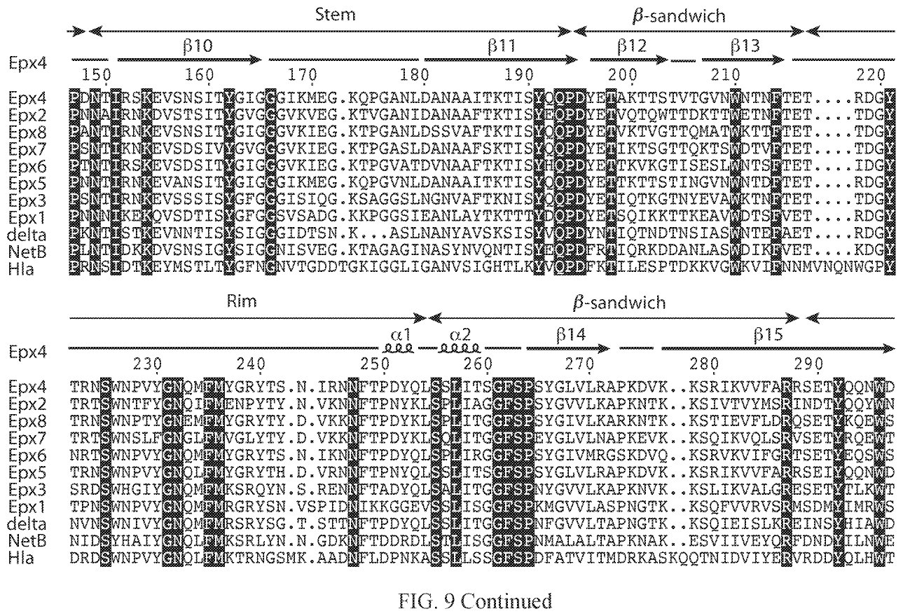

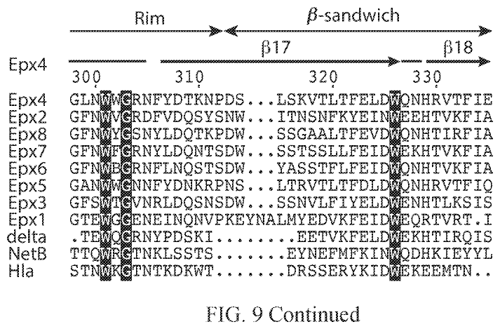

Disclosed herein, in some aspects, are Epx polypeptides (e.g. Exp1-Exp8) and variants thereof. In some embodiments, Exp polypeptide may form pores. In some embodiments, Exp polypeptide may form homo-octameric pores. In some embodiments, the pores comprise a Top domain, a Cap domain, and Rim domain, a Stem domain as shown in FIG. 2A and FIG. 9. The Stem domain may insert into the membrane of a cell creating a pore. The Rim domain may also interact with a cell membrane. Results suggest that the Top domain may be required for pore toxicity. The poor is about 18 angstroms in diameter and thus is a sufficient size to allow translocation of biological polymers (e.g. DNA or RNA). Results also suggest that the Exp polypeptide may be immunogenic and thus may be used as an adjuvants or in vaccines. In some embodiments, the Exp polypeptide binds to MHC class I receptor, which plays a role in antigen presentation in the immune response. Thus, the Epx polypeptide may be used in treating diseases associated with major histocompatibility complex (MHC) class I dysregulation.

### Compositions

As used herein, the term “Enterococci toxin (Epx) polypeptide” encompasses any polypeptide or fragment from a Epx polypeptide. In some embodiments, the term Enterococci toxin (Epx) polypeptide refers to a full-length Epx polypeptide. In some embodiments, the term Enterococci toxin (Epx) polypeptide refers to a fragment of the Epx polypeptide that can form a pore. In some embodiments, the term Enterococci toxin (Epx) polypeptide simply refers to a fragment of the Epx polypeptide, without requiring the fragment to have any specific function or activity. In some embodiments, the Enterococci toxin (Epx) polypeptide does not comprise a signal sequence. Other terms that may be used throughout the present disclosure for Enterococci toxin (Epx) polypeptide may be Epx polypeptide. It is to be understood that these terms are used interchangeably.

As used herein, the term “isolated Enterococci toxin (Epx) polypeptide” may encompass any Epx polypeptide that has been extracted from a cell or produced in vitro. For example, an isolated Enterococci toxin (Epx) polypeptide may be purified from an Enterococci. In some embodiments, an isolated Enterococci toxin (Epx) polypeptide may be a purified from a cell that is engineered to express an Epx polypeptide (e.g. E. Coli). In some embodiments, an isolated Enterococci toxin (Epx) polypeptide may be able to form a pore. In some embodiments, an isolated Enterococci toxin (Epx) polypeptide may be able to form a pore that is toxic. In some embodiments, an isolated Enterococci toxin (Epx) polypeptide may be able to form a pore that is toxic to mammalian cells. In some embodiments, an isolated Enterococci toxin (Epx) polypeptide may be able to form a pore that is toxic to human cells. Other terms that may be used throughout the present disclosure for isolated Enterococci toxin (Epx) polypeptide may be isolated Epx polypeptide. It is to be understood that these terms are used interchangeably.

In some aspects, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to any one of SEQ ID NOs: 1-8. In some embodiments, the present application discloses isolated Enterococci toxin (Epx) polypeptide comprising the amino acid sequence of any one of SEQ ID NOs: 1-8. In some embodiments, the present application discloses isolated Enterococci toxin (Epx) polypeptide, consisting of the amino acid sequence of any one of SEQ ID NOs: 1-8.

In some embodiments, the present application discloses an isolated Enterococci toxin (Epx) polypeptide comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 1, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 1, wherein the amino acid sequence is not 100% identical to any one of SEQ ID NOs: 1-8.

In some embodiments, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 2, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 2, wherein the amino acid sequence is not 100% identical to any one of SEQ ID NOs: 1-8.

In some embodiments, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 3, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 3, wherein the amino acid sequence is not 100% identical to any one of SEQ ID NOs: 1-8.

In some embodiments, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 4, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 4, wherein the amino acid sequence is not 100% identical to any one of SEQ ID NOs: 1-8.

In some embodiments, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 5, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 5, wherein the amino acid sequence is not 100% identical to any one of SEQ ID NOs: 1-8.

In some embodiments, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 6, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 6, wherein the amino acid sequence is not 100% identical to any one of SEQ ID NOs: 1-8.

In some embodiments, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 7, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 7, wherein the amino acid sequence is not 100% identical to any one of SEQ ID NOs: 1-8.

In some embodiments, the present application discloses isolated Enterococci toxin (Epx) polypeptide, comprising an amino acid sequence that is at least 85% identical (e.g. at least 90% identical, at least 95% identical, at least 96% identical, at least 97% identical, at least 98% identical, at least 99% identical, at least 99.5% identical to SEQ ID NO: 8, wherein the amino acid sequence is not 100% identical to any one of SEQ ID NOs: 1-8. In some embodiments, the present application discloses isolated Enterococci toxin (Epx) polypeptide, comprising an amino acid sequence that is at least 95% identical to SEQ ID NO: 8, wherein the amino acid sequence is not 100% identical to any one of SEQ ID NOs: 1-8.

In some embodiments, the isolated Epx polypeptide does not comprise a signal sequence. In some embodiments, the isolated Epx polypeptide comprises a signal sequence. In some embodiments, the signal sequence is a naturally occurring signal sequence. In some embodiments, the signal sequence is a synthetic signaling sequence. In some embodiments, the signal sequence is selected from the group consisting of SEQ ID NOs: 26-33. Peptide signal sequences are well known in the art as described at signalpeptide.de/.

In some embodiments, the present application discloses a pore comprising the isolated Epx polypeptide described herein. As described here, a “pore” may refer to protein complex that when inserted into a membrane produces a hole in the membrane (e.g. see FIG. 2E). In some embodiments, the pore comprises transmembrane domain (e.g. stem domain), a Rim domain comprising aromatic amino acids that may interact with the membrane, a Cap domain and a Top domain (e.g. see FIG. 2A). In some embodiments, the pore is about 18 Angstroms in diameter. In some embodiments, the pore diameter of sufficient size for the translocation of biological polymers (e.g. DNA, RNA, or polypeptide). In some embodiments, the pore is toxic to mammalian cells. Other terms that may be used throughout the present disclosure for pore including nanopore, Exp polypeptide pore, and Exp polypeptide nanopore. It is to be understood that these terms are used interchangeably.

In some embodiments, the instant application discloses an apparatus comprising the isolated pore described herein and a membrane. In some embodiments, the pore is disposed in the membrane. Any suitable membrane may be used in the apparatus. Suitable membranes are well known in the art. For example, as described in Gonzalez-Perez et al., Langmuir, 2009, 25, 10447-10450 and US Patent Publication 20210087621, each of which are incorporated by reference in its entirety. In some embodiments, the suitable membrane is a amphiphilic membrane. In some embodiments, the amphiphilic membrane comprises amphiphilic molecules (e.g. phospholipids that comprises polar and nonpolar regions). In some embodiments, the membrane comprises block copolymers (e.g. molecules that comprise two or more monomers polymerized together). In some embodiments, the membrane is a lipid monolayer. In some embodiments, the membrane is a lipid bilayer.

Methods for inserting pores into membranes (e.g. amphiphilic membranes) are well known in the art and include suspending purified pores in a solution containing a triblock copolymer membrane such that the pore may diffuse into the membrane and direct insertion using the “pick and place” method as described in M. A. Holden, H. Bayley. J. Am. Chem Soc. 2005, 127, 6502-6503 and International Application No, PCT/GB2006/001057 (published as WO 2006/100484), both of which are incorporated by reference in their entirety. As used herein, the term “modified Epx polypeptide” may refer to an Epx polypeptide that has been modified to comprise a mutation. In some embodiments, the modified Epx polypeptide comprises a mutation corresponding to the Top domain of the Epx polypeptide. In some embodiments, a modified Epx polypeptides comprises 1 or 2 mutations. In some embodiments, a mutation may be an amino acid substitution, an insertion, or a deletion. In some embodiment, a charged amino acid (e.g. lysine, arginine, histidine, glutamate or aspartate) is substituted with a neutral amino acid or an amino acid of the opposite charge. Positive amino acids may include, but are not limited to, lysine, arginine, and histidine. Negative amino acids may include, but are not limited to, glutamate and aspartate. Neutral amino acids include, but are not limited to, serine, threonine, asparagine, glutamine, cysteine, glycine, proline, alanine, valine, leucine, isoleucine, methionine, phenylalanine, tyrosine, and tryptophan. Hydrophilic uncharged amino acids may include, but are not limited to serine, threonine, asparagine, and glutamine. In some embodiments, hydrophilic uncharged amino acids may also include glycine. Hydrophobic uncharged amino acids may include, but are not limited to, cysteine, proline, alanine, valine, leucine, isoleucine, methionine, phenylalanine, tyrosine, and tryptophan.

In some embodiments, the modified Epx polypeptide comprises an amino acid substitution at a position corresponding to K50 of SEQ ID NO: 9. In some embodiments, the modified Epx polypeptide comprises a neutral amino acid substitution at a position corresponding to K50 of SEQ ID NO: 9. In some embodiments, the modified Epx polypeptide comprises a hydrophilic uncharged amino acid substitution at a position corresponding to K50 of SEQ ID NO: 9. In some embodiments, the modified Epx polypeptide comprises a hydrophobic uncharged amino acid substitution at a position corresponding to K50 of SEQ ID NO: 9.

In some embodiments, the modified Epx polypeptide comprises an amino acid substitution at a position corresponding to K56 of SEQ ID NO: 9. In some embodiments, the modified Epx polypeptide comprises a hydrophilic uncharged amino acid substitution at a position corresponding to K56 of SEQ ID NO: 9. In some embodiments, the modified Epx polypeptide comprises a hydrophobic uncharged amino acid substitution at a position corresponding to K56 of SEQ ID NO: 9.

In some embodiments, the modified Epx polypeptide comprises an amino acid substitution at a position corresponding to K50 and K56 of SEQ ID NO: 9. In some embodiments, the modified Epx polypeptide comprises a hydrophilic uncharged amino acid substitution at a position corresponding to K50 and K56 of SEQ ID NO: 9. In some embodiments, the modified Epx polypeptide comprises a hydrophobic uncharged amino acid substitution at a position corresponding to K50 and K56 of SEQ ID NO: 9.

In some embodiments, the modified Epx polypeptides comprises an amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to any one of SEQ ID NOS: 9-16 and an amino acid substitution at a position corresponding to K50 and/or K56 of SEQ ID NO: 9. In some embodiments, the modified Epx polypeptides comprises modified Epx polypeptides comprising an amino acid sequence that is at least 95% identical to any one of SEQ ID NOs: 9-16 and an amino acid substitution at a position corresponding to K50 and/or K56 of SEQ ID NO: 9. In some embodiments, the modified Epx polypeptides comprises an amino acid sequence that is at least 99% identical to any one of SEQ ID NOs: 9-16 and an amino acid substitution at a position corresponding to K50 and/or K56 of SEQ ID NO: 9. In some embodiments, the modified Epx polypeptides comprise the amino acid sequence of any one of SEQ ID NOs: 9-16 and an amino acid substitution at a position corresponding to K50 and/or K56 of SEQ ID NO: 9.

In some embodiments, the modified Exp polypeptide is a modified Epx1 polypeptide comprising a K50A substitution in SEQ ID NO: 9. In some embodiments, the modified Epx1 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 17. In some embodiments, the modified Epx1 polypeptide comprises amino acid sequence that is at least 95% identical to SEQ ID NO: 17. In some embodiments, the modified Epx1 polypeptide comprises amino acid sequence of SEQ ID NO: 17. In some embodiments, the modified Epx1 polypeptide consists of the amino acid sequence of SEQ ID NO: 17.

In some embodiments, the modified Exp polypeptide is a modified Epx1 polypeptide comprising a K50E substitution in SEQ ID NO: 9. In some embodiments, the modified Epx 1 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 18. In some embodiments, the modified Epx1 polypeptide comprises amino acid sequence that is at least 95% identical to SEQ ID NO: 18. In some embodiments, the modified Epx1 polypeptide comprises amino acid sequence of SEQ ID NO: 18. In some embodiments, the modified Epx 1 polypeptide consists of the amino acid sequence of SEQ ID NO: 18.

In some embodiments, the modified Exp polypeptide is a modified Epx2 polypeptide comprising a K54E substitution in SEQ ID NO: 9.

In some embodiments, the modified Exp polypeptide is a modified Epx1 polypeptide comprising a K50E substitution and a K54E substitution in SEQ ID NO: 9. In some embodiments, the modified Epx1 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 19. In some embodiments, the modified Epx 1 polypeptide comprises amino acid sequence that is at least 95% identical to SEQ ID NO: 19. In some embodiments, the modified Epx1 polypeptide comprises amino acid sequence of SEQ ID NO: 19. In some embodiments, the modified Epx1 polypeptide consists of the amino acid sequence of SEQ ID NO: 19.

In some embodiments, the modified Exp polypeptide is a modified Epx2 polypeptide comprising a K50A substitution in SEQ ID NO: 10. In some embodiments, the modified Epx2 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 20. In some embodiments, the modified Epx2 polypeptide comprises amino acid sequence that is at least 95% identical to SEQ ID NO: 20. In some embodiments, the modified Epx1 polypeptide comprises amino acid sequence of SEQ ID NO: 20. In some embodiments, the modified Epx1 polypeptide consists of the amino acid sequence of SEQ ID NO: 20.

In some embodiments, the modified Exp polypeptide is a modified Epx2 polypeptide comprising a K50E substitution in SEQ ID NO: 10. In some embodiments, the modified Epx2 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 21. In some embodiments, the modified Epx2 polypeptide comprises amino acid sequence that is at least 95% identical to SEQ ID NO: 21. In some embodiments, the modified Epx2 polypeptide comprises amino acid sequence of SEQ ID NO: 21. In some embodiments, the modified Epx2 polypeptide consists of the amino acid sequence of SEQ ID NO: 21.

In some embodiments, the modified Exp polypeptide is a modified Epx2 polypeptide comprising a K56E substitution in SEQ ID NO: 10.

In some embodiments, the modified Exp polypeptide is a modified Epx2 polypeptide comprising a K50E substitution and a K56E substitution in SEQ ID NO: 10. In some embodiments, the modified Epx2 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 22. In some embodiments, the modified Epx2 polypeptide comprises amino acid sequence that is at least 95% identical to SEQ ID NO: 22. In some embodiments, the modified Epx2 polypeptide comprises amino acid sequence of SEQ ID NO: 22. In some embodiments, the modified Epx2 polypeptide consists of the amino acid sequence of SEQ ID NO: 22.

In some embodiments, the modified Exp polypeptide is a modified Epx4 polypeptide comprising a K51A substitution in SEQ ID NO: 12. In some embodiments, the modified Epx4 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 23. In some embodiments, the modified Epx4 polypeptide comprises amino acid sequence that is at least 95% identical to SEQ ID NO: 23. In some embodiments, the modified Epx4 polypeptide comprises amino acid sequence of SEQ ID NO: 23. In some embodiments, the modified Epx4 polypeptide consists of the amino acid sequence of SEQ ID NO: 23.

In some embodiments, the modified Exp polypeptide is a modified Epx4 polypeptide comprising a K51E substitution in SEQ ID NO: 12. In some embodiments, the modified Epx4 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 24. In some embodiments, the modified Epx4 polypeptide comprises amino acid sequence that is at least 95% identical to SEQ ID NO: 24. In some embodiments, the modified Epx4 polypeptide comprises amino acid sequence of SEQ ID NO: 24. In some embodiments, the modified Epx4 polypeptide consists of the amino acid sequence of SEQ ID NO: 24.

In some embodiments, the modified Exp polypeptide is a modified Epx4 polypeptide comprising a K57E substitution in SEQ ID NO: 12.

In some embodiments, the modified Exp polypeptide is a modified Epx4 polypeptide comprising a K51E substitution and a K57E substitution in SEQ ID NO: 12. In some embodiments, the modified Epx4 polypeptide comprises amino acid sequence that is at least 85% identical (e.g. at least 85% identical, at least 90% identical, at least 95% identical, at least 96% identical, at least 97% identical, at least 98% identical, at least 99% identical, at least 99.5% identical, or at least 99.9% identical) to SEQ ID NO: 25. In some embodiments, the modified Epx4 polypeptide comprises amino acid sequence that is at least 95% identical to SEQ ID NO: 25. In some embodiments, the modified Epx4 polypeptide comprises amino acid sequence of SEQ ID NO: 25. In some embodiments, the modified Epx4 polypeptide consists of the amino acid sequence of SEQ ID NO: 25.

In some embodiments, the present application discloses compositions comprising an Epx polypeptide as described herein or a fragment thereof.

A “fragment thereof” of an Epx polypeptide may refer to any portion of a Epx polypeptide. In some embodiments, a fragment of an Epx polypeptide comprises a peptide that is part of the Top domain, Cap domain, Rim domain, or Stem domain of the Epx polypeptide (see FIG. 2B). In some embodiments, a fragment of an Epx polypeptide is an epitope for antibody generation and/or binding. In some embodiments, a fragment of an Epx polypeptide can induce an immune response. In some embodiments, a fragment of an Epx polypeptide is at least 10 sequential amino acids (e.g. at least 15, at least 20, or at least 50) of any one of SEQ ID NOs: 1-25. In some embodiments, a fragment of an Epx polypeptide comprises at least 10-15, 10-20, 10-30, 10-50, 15-20, 15-30, 15-50, 20-30, 20-50, or 30-50 sequential amino acids of any one of SEQ ID NOs: 1-25.

In some embodiments, the composition comprises an Epx polypeptide or fragment thereof and an antigen. In some embodiments, the antigen is a viral antigen, a bacterial antigen, a cancer antigen, a fungal antigen, or a parasitic antigen.

In some embodiments, the viral antigen is from a virus selected from the group consisting of Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus Merkel cell polyomavirus, Mokola virus, Molluscum contagiosum virus, Monkeypox virus Mumps virus, Murray valley encephalitis virus, New York virus, Nipah virus, Norwalk virus Oï¿½ nyong-nyong virusm, Orf virus, Oropouche virus, Pichinde virus, Poliovirus, Punta toro, phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus

Simian virus 5, Sindbis virus, Southampton virus, St. Louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus Western equine encephalitis virus, WU polyomavirus, West Nile virus, Yaba monkey tumor virus, Yaba-like disease virus, Yellow fever virus or Zika virus.

In some embodiments, the bacterial antigen is from a bacteria selected from the group consisting of pneumococcal, meningococcal, typhoid, cholera, tetanus, haemophilus b, anthrax, Methicillin-resistant Staphylococcus aureus (MRSA), Clostridium difficile, Gonorrhea, Bubonic plague, Syphilis, E. coli, Salmonella, Botulism, Klebsiella pneumoniae, Pseudomonas aeruginosa, Streptococcus pneumoniae, Heliobacter pylori (H. pylori), Vibrio vulnificus, Achromobacter xylosoxidans, Acinetobacter baumannii, Actinomyces, Actinomyces israelii, Aeromonas species, Bacillus species, Bacteroides fragilis, Bacteroides melaninogenicus, Bartonella species, Bordetella pertussis, Borrelia species, Brucella species, Burkholderia species

Campylobacter, Capnocytophaga species, Chlamydophila pneumoniae, Chlamydophila psittaci Citrobacter species, Clostridium species, Corynebacterium species, Coxiella burnetii, Ehrlichia species, Eikenella corrodens, Enterobacter species, Enterococcus faecalis, Enterococcus faecium, Escherichia coli, Francisella tularensis, Fusobacterium necrophorum, Gardnerella vaginalis, Haemophilus species, Helicobacter Pylori, Klebsiella species, Lactobacillus species, Legionella species, Leptospira species, Listeria monocytogenes, Moraxella catarrhalis, Morganella species, Mycoplasma pneumonia, Neisseria species, Nocardia species, Pasteurella multocida, Peptostreptococcus species, Porphyromonas gingivalis, Propionibacterium acnes

Proteus species, Providencia species, Pseudomonas aeruginosa, Salmonella species, Serratia marcescens, Shigella species, Staph epidermidis, Staph hominis, Staph. Haemolyticus, Staphylococcus aureus, Staphylococcus saprophyticus, Stenotrophomonas maltophilia Streptococcus agalactiae, Streptococcus anginosus group, Streptococcus pneumoniae Streptococcus pyogenes (Groups A, B, C, G, F), Treponema pallidum, or Vibrio species.

In some embodiments, the cancer antigen is from a cancer selected from the group consisting of, Acute Lymphoblastic Leukemia (ALL), Acute Myeloid Leukemia (AML), Adrenocortical Carcinoma, Kaposi Sarcoma, AIDS-Related Lymphoma, Primary CNS Lymphoma, Anal Cancer, Appendix Cancer, Astrocytomas, Atypical Teratoid/Rhabdoid Tumor, Brain Cancer, Basal Cell Carcinoma of the Skin-see Skin Cancer, Bile Duct Cancer, Bladder Cancer, Bone Cancer (includes Ewing Sarcoma and Osteosarcoma and Malignant Fibrous Histiocytoma), Breast Cancer, Bronchial Tumors, Burkitt Lymphoma, Non-Hodgkin Lymphoma, Carcinoid Tumor, Cardiac Tumors, Atypical Teratoid/Rhabdoid Tumor, Medulloblastoma and Other CNS Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Embryonal Tumors, Medulloblastoma, Endometrial Cancer, Ependymoma, Esophageal Cancer, Esthesioneuroblastoma, Ewing Sarcoma, Extracranial Germ Cell Tumor, Childhood, Extragonadal Germ Cell Tumor, Eye Cancer, Intraocular Melanoma, Retinoblastoma, Fallopian Tube Cancer, Gallbladder Cancer, Gastric Cancer, Gastrointestinal Carcinoid Tumor, Gastrointestinal Stromal Tumors (GIST), Germ Cell Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis, Langerhans Cell Hodgkin Lymphoma, Hypopharyngeal Cancer, Intraocular Melanoma, Islet Cell Tumors, Pancreatic Neuroendocrine Tumors, Kaposi Sarcoma, Kidney Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Melanoma, Melanoma, Intraocular cancer, Merkel Cell Carcinoma, Mesothelioma, Malignant, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelogenous Leukemia, Chronic (CML), Myeloid Leukemia, Acute (AML), Myeloproliferative Neoplasms, Chronic Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell Cancer, Retinoblastoma Rhabdomyosarcoma, Childhood Soft Tissue Sarcoma, Salivary Gland Cancer, Sarcoma, Childhood Rhabdomyosarcoma, Childhood Vascular Tumors, Ewing Sarcoma, Kaposi Sarcoma Osteosarcoma, Soft Tissue Sarcoma, Uterine Sarcoma, Sezary Syndrome (Lymphoma), Skin Cancer, Small Cell Lung Cancer, Small Intestine Cancer, Squamous Cell Carcinoma of the Skin, Squamous Neck Cancer with Occult Primary Metastatic, Stomach Cancer, T-Cell Lymphoma Cutaneous, Testicular Cancer, Throat Cancer, Nasopharyngeal Cancer, Oropharyngeal Cancer, Hypopharyngeal Cancer, Thymoma and Thymic Carcinoma, Thyroid Cancer, Tracheobronchial Tumors, Transitional Cell Cancer of the Renal Pelvis and Ureter, Carcinoma of Ureter and Renal Pelvis, Transitional Cell Cancer, Urethral Cancer, Uterine Cancer, Endometrial, Uterine Sarcoma, Vaginal Cancer, Vascular Tumors, Vulvar Cancer, Wilms Tumor, or child kidney tumors.

In some embodiments, the fungal antigen is from a fungus selected from the group consisting of Aspergillus, Blastomyces, Candidiasis, Candida auris, Coccidioides, C. neoformans, C. gattii, Histoplasma, Mucormycosis, Pneumocystis jirovecii, Sporothrix, Sporothrix brasiliensis, Paracoccidioides and Talaromyces marneffei.

In some embodiments, the parasitic antigen is from a parasite selected from the group consisting of, round worms, flat worms, malaria, Giardia, Toxoplasma gondii, E. vermicularis, Trypanosoma cruzi, Echinococcus, Taenia solium, Toxocara canis, Toxocara cati, Trichomonas vaginalis, and Entamoeba histolytica.

In some embodiments, the antigen is a peptide antigen. In some embodiments, the peptide antigen may be from any one of the viruses, bacteria, cancers, fungi, or parasites disclosed herein. In some embodiments, the peptide antigen comprises at least 5 amino acids (e.g. at least 10 amino acids, at least 15 amino acids, at least 20 amino acids, at least 50 amino acids, or at least 100 amino acids).

In some embodiments, the Epx polypeptide and the antigen are conjugated. In some embodiments, the Epx polypeptide and the antigen are covalently conjugated. In some embodiments, the Epx polypeptide and the antigen are covalently conjugated using a crosslinking reagent. In some embodiments, the crosslinking reagent is selected from the group consisting of a homobifunctional crosslinking reagents (e.g. BMOE and DTME), heterobifunctional crosslinking reagents (m-Maleimidobenzoyl-N-hydroxysuccinimide ester, N-γ-Maleimidobutyryloxysuccinimide ester, N-(ε-Maleimidocaproyloxy) succinimide ester) and N-(ε-Maleimidocaproyloxy) sulfo succinimide ester, and photoreactive crosslinking reagents (e.g. aryl-azides and diazirines). In some embodiments, the Epx polypeptide and the antigen are non-covalently conjugated (e.g. via hydrophobic interactions or an avidin-biotin interaction).

In some embodiments, the Epx polypeptide and a peptide antigen are conjugated using a peptide bond (e.g., to form a fusion protein). In some embodiments, conjugation using a peptide bond is accomplished by encoding the Epx polypeptide and a peptide antigen on the same transcript. Thus, in some embodiments, when the transcript is translated, a fusion protein comprising the Epx polypeptide and the peptide antigen is produced. In some embodiments, the fusion protein from N-terminal to C-terminal comprises the peptide antigen then the modified Epx polypeptide or a fragment thereof. In some embodiments, the fusion protein from N-terminal to C-terminal comprises the n-terminal of the modified Epx polypeptide or a fragment thereof then the peptide antigen.

In some embodiments, the composition is an immunogenic composition. An “immune genic composition” as described herein, may refer to a composition that is expected to induce or does induce an immune response in a subject. In some embodiments, an immunogenic composition may induce an innate immune response. In some embodiments, an immunogenic composition may induce an adaptive immune response against an antigen (e.g. an antigen from a pathogen described herein).

In some embodiments, the composition (e.g. comprising a modified Epx polypeptide, conjugate or fusion protein thereof) comprises a vaccine. In some embodiments, the composition comprises an Epx polypeptide that cannot form a pore and/or is not toxic (e.g., a modified Epx polypeptide. In some embodiments, the vaccine induces an immune response against the Epx polypeptide. In some embodiments, the immune response generates antibodies against the Epx polypeptide. In some embodiments, the vaccine provides protection against pathogens expressing the Epx polypeptide (e.g. Enterococci). In some embodiments, the vaccine further comprises an adjuvant. In some embodiments, the adjuvant is the modified Exp polypeptide. In some embodiments, the modified Epx polypeptide is an antigen and an adjuvant. In some embodiments, the vaccine induces an immune response against an antigen (e.g. an antigen described herein). In some embodiments, the vaccine protects against infection by a pathogen that expresses the antigen.

In some embodiments, the composition (e.g. comprising a modified Epx polypeptide, conjugate or fusion protein thereof) is an adjuvant. In some embodiments, the composition comprises an Epx polypeptide that cannot form a pore and/or is not toxic. In some embodiments, the adjuvant induces an immune response in a subject. In some embodiments, the adjuvant is added to a vaccine to induce an immune response against an antigen. In some embodiments, the adjust induces an innate immune response. In some embodiments, the adjuvant induces an adaptive immune response.

TABLE 1
Epx polypeptide and signal sequences

| SEQ | | |
|---|---|---|
| ID | | |
| NO | Description | Sequence |
| 1 | Epx1 w/o | QDVQVFADNPVLGTEVTFEKDENGRIVKIISKNQYRITIYNSVD |
| | signal | AGDTPNDASVSLDVDFIDDKNSGEMGAVASINTFIPSGLRYVEG |
| | sequence | YKYKGVTNPIYKNLSAGMLWPKKYRVEVVNIPIDQATKIITATP |
| | | NNNIKEKQVSDTISYGFGGSVSADGKKPGGSIEANLAYTKTTTY |
| | | DQPDYETSQIKKTTKEAVWDTSFVETRDGYTPNSWNPVYGNQ |
| | | MFMRGRYSNVSPIDNIKKGGEVSSLISGGFSPKMGVVLASPNGT |
| | | KKSQFVVRVSRMSDMYIMRWSGTEWGGENEINQNVPKEYNAL |
| | | MYEDVKFEIDWEQRTVRTI |
| 2 | Epx2 w/o | SFNEDNILGTTTKETDEDGKTKTIITVKDRQIESYTSSDSATKEN |
| | signal | NTSLTVNANFLNDKYSSDLTTIMSLNGFIPSGKQYYFPPNNTWV |
| | sequence | GNMYWPSQYKTTVSSESLDQTVKIINTTPNNAIRNKDVSTSITY |
| | | GVGGGVKVEGKTVGANIDANAAFTKTISYEQPDYETVQTQWT |
| | | TDKTTWETNFTETTDGYTRTSWNTFYGNQIFMENPYTYNVKN |
| | | NFTPNYKLSPLIAGGFSPSYGVVLKAPKNTKKSIVTVYMSRIND |
| | | TYQQYWNGFNWVGRDFVDQSYSNWITNSNFKYEINWEEHTV |
| | | KFIA |
| 3 | Epx3 w/o | ASEEDTFAGNVSTETDSNGTEKNIITSNGKQIENYNSTDSNTKK |
| | signal | NNSSITINANFIEDTHSSEKTTVLSVNGFVPSGRKFIYPKNNTWE |
| | sequence | ASMLWPSSYKATIVNESIDSPVKITNSTPSNTIRNKEVSSSISYGF |
| | | GGGISIQGKSAGGSLNGNVAFTKNISYQQPDYETIQTKGTNYEV |
| | | AWKTNFTETTDGYSRDSWHGIYGNQMFMKSRQYNSRENNFTA |
| | | DYQLSALITGGFSPNYGVVLKAPKNVKKSLIKVALGRESETYTL |
| | | KWTGFSWTGVNRLDQSNSDWSSNVLFIYELDWENHTLKSIS |
| 4 | Epx4 w/o | SEDNIIGTTTQEIDEHGNVKTIITVKNQQIESYTSTDSGTAKNRST |
| | signal | LTVNANFLNDKYSNELTTILSLNGFIPSGRKFIFPKNNTLKGEML |
| | sequence | WPQRYSTAVYNIPLDKSVKITNSTPDNTIRSKEVSNSITYGIGGG |
| | | IKMEGKQPGANLDANAAITKTISYQQPDYETAKTTSTVTGVNW |
| | | NTNFTETRDGYTRNSWNPVYGNQMFMYGRYTSNIRNNFTPDY |
| | | QLSSLITSGFSPSYGLVLRAPKDVKKSRIKVVFARRSETYQQNW |
| | | DGLNWWGRNFYDTKNPDSLSKVTLTFELDWQNHRVTFIE |
| 5 | Epx5 w/o | ADFSEDNIIGTTTHEIDEHGNVKTIITVKNQQVESYTSTDSGTAK |
| | signal | NRSTMTVNANFLNDKYSNELTTILSENGFIPSGRKFIFPKNNTW |
| | sequence | KGEMMWPQRYSTAVYNESLDKPVKISDSTPNNTIRNKEVANSI |
| | | TYGIGGGIKMEGKQPGVNLDANAAITKTISYQQPDYETTKTTST |
| | | INGVNWNTDFTETRDGYTRNSWNPVYGNQLFMYGRYTHDVR |
| | | NNFTPNYQLSSLITSGFSPSYGLVLRAPKDVKKSRIKVVFARRSE |
| | | IYQQNWDGANWWGNNFYDNKRPNSLTRVTLTFDLDWQNHRV |
| | | TFIQ |
| 6 | Epx6 w/o | ADFNEDNILGTTTVERDEQGNIKTIITVKNRTVESYSSNDTATSN |
| | signal | NYDMTATLNATFLNDKYSNNLTTILSLNGFIPSGRKFSFPSNNS |
| | sequence | WLGSMLWPEQYSIKVMSESLDKPVKITDSTPTNTIRSKEVSDSIT |
| | | YGIGGGVKIEGKTPGVATDVNAAFTKTISYHQPDYETTKVKGTI |
| | | SESLWNTSFTETIDGYNRTSWNPVYGNQMFMYGRYTSNIKNNF |
| | | TPDYQLSPLIRGGFSPSYGIVMRGSKDVQKSRVKVIFGRTSETYE |
| | | QSWSGFNWVGRNFLNQSTSDWYASSTFLFEIDWQNHTVKFIA |
| 7 | Epx7 w/o | ADFNESNTIGTTTTEKDEDGKTKTIITVKDRQIESYTSTDAATSS |
| | signal | NNSSVSINATFFNDRYSDKLTTLLSFKGFIPSGRRFTFPPRNSWL |
| | sequence | GFMLWPYKYSTTITNISLDKPVKIIESTPSNTIKNKEVSDSIVYGV |
| | | GGGVKLEGKTPGASLDANAAFSKTISYQQPDYETIKTSGTTQKT |
| | | SWDTVFTETTDGYTRTSWNSLFGNGLFMVGLYTYDVKKNFTP |
| | | DYKLSQLITGGFSPEYGLVLNAPKEVKKSQIKVQLSRVSETYRQ |
| | | EWTGFNWFGRNYLDQNTSDWSSTSSLLFEIDWEKHTVKFIA |
| 8 | Epx8 w/o | ADYNEDNILGTTTTETDENGNIKTIITVKNRQIETYNSSDSATTS |
| | signal | NNSSITVNGTFFNDKYSQELTTILSVSGFVPSGRKFIFPPHNTWV |
| | sequence | GSMLWPVQYSTTVKNSSKDNTVKISDSTPANTIRNKEVSNSITY |
| | | GIGGGVKIEGKTPGANLDSSVAFTKTISYQQPDYETVKTVGTTQ |
| | | MATWKTTFTETTDGYTRNSWNPTYGNEMFMYGRYTYDVKKN |
| | | FTPDYKLSPLITGGFSPSYGIVLKARKNTKKSTIEVFLDRQSETY |
| | | KQEWSGFNWYGSNYLDQTKPDWSSGAALTFEVDWQNHTIRFI |
| | | A |
| 9 | Epx1 with | MLKLKKIFLFTMISVSLCCGFGQDVQVFADNPVLGTEVTFEKDE |
| | signal | NGRIVKIISKNQYRITIYNSVDAGDTPNDASVSLDVDFIDDKNSG |
| | sequence | EMGAVASINTFIPSGLRYVEGYKYKGVTNPIYKNLSAGMLWPK |
| | | KYRVEVVNIPIDQATKIITATPNNNIKEKQVSDTISYGFGGSVSA |
| | | DGKKPGGSIEANLAYTKTTTYDQPDYETSQIKKTTKEAVWDTS |
| | | FVETRDGYTPNSWNPVYGNQMFMRGRYSNVSPIDNIKKGGEV |
| | | SSLISGGFSPKMGVVLASPNGTKKSQFVVRVSRMSDMYIMRWS |
| | | GTEWGGENEINQNVPKEYNALMYEDVKFEIDWEQRTVRTI |
| 10 | Epx2 with | MKCMNLKKIFLFNTVIISILLVSSPVSASFNEDNILGTTTKETDED |
| | signal | GKTKTIITVKDRQIESYTSSDSATKENNTSLTVNANFLNDKYSS |
| | sequence | DLTTIMSLNGFIPSGKQYYFPPNNTWVGNMYWPSQYKTTVSSE |
| | | SLDQTVKIINTTPNNAIRNKDVSTSITYGVGGGVKVEGKTVGAN |
| | | IDANAAFTKTISYEQPDYETVQTQWTTDKTTWETNFTETTDGY |
| | | TRTSWNTFYGNQIFMENPYTYNVKNNFTPNYKLSPLIAGGFSPS |
| | | YGVVLKAPKNTKKSIVTVYMSRINDTYQQYWNGFNWVGRDF |
| | | VDQSYSNWITNSNFKYEINWEEHTVKFIA |
| 11 | Epx3 with | MKLKFALSVLSLSTILLSASPVLASEEDTFAGNVSTETDSNGTEK |
| | signal | NIITSNGKQIENYNSTDSNTKKNNSSITINANFIEDTHSSEKTTVL |
| | sequence | SVNGFVPSGRKFIYPKNNTWEASMLWPSSYKATIVNESIDSPVK |
| | | ITNSTPSNTIRNKEVSSSISYGFGGGISIQGKSAGGSLNGNVAFTK |
| | | NISYQQPDYETIQTKGTNYEVAWKTNFTETTDGYSRDSWHGIY |
| | | GNQMFMKSRQYNSRENNFTADYQLSALITGGFSPNYGVVLKA |
| | | PKNVKKSLIKVALGRESETYTLKWTGFSWTGVNRLDQSNSDW |
| | | SSNVLFIYELDWENHTLKSIS |
| 12 | Epx4 with | MIKLKKKNYKIWLSIAVLSAMLTTVPVFANFSEDNIIGTTTQEID |
| | signal | EHGNVKTIITVKNQQIESYTSTDSGTAKNRSTLTVNANFLNDKY |
| | sequence | SNELTTILSLNGFIPSGRKFIFPKNNTLKGEMLWPQRYSTAVYNI |
| | | PLDKSVKITNSTPDNTIRSKEVSNSITYGIGGGIKMEGKQPGANL |
| | | DANAAITKTISYQQPDYETAKTTSTVTGVNWNTNFTETRDGYT |
| | | RNSWNPVYGNQMFMYGRYTSNIRNNFTPDYQLSSLITSGFSPSY |
| | | GLVLRAPKDVKKSRIKVVFARRSETYQQNWDGLNWWGRNFY |
| | | DTKNPDSLSKVTLTFELDWQNHRVTFIE |
| 13 | Epx5 with | MVKLKKKNHKIWISIAVLSAMLTTVPVFADFSEDNIIGTTTHEID |
| | signal | EHGNVKTIITVKNQQVESYTSTDSGTAKNRSTMTVNANFLNDK |
| | sequence | YSNELTTILSFNGFIPSGRKFIFPKNNTWKGEMMWPQRYSTAVY |
| | | NESLDKPVKISDSTPNNTIRNKEVANSITYGIGGGIKMEGKQPG |
| | | VNLDANAAITKTISYQQPDYETTKTTSTINGVNWNTDFTETRDG |
| | | YTRNSWNPVYGNQLFMYGRYTHDVRNNFTPNYQLSSLITSGFS |
| | | PSYGLVLRAPKDVKKSRIKVVFARRSEIYQQNWDGANWWGNN |
| | | FYDNKRPNSLTRVTLTFDLDWQNHRVTFIQ |
| 14 | Epx6 with | MKIFRKSISKRIFFCAVLSGLMYTSPVQADFNEDNILGTTTVERD |
| | signal | EQGNIKTIITVKNRTVESYSSNDTATSNNYDMTATLNATFLNDK |
| | sequence | YSNNLTTILSLNGFIPSGRKFSFPSNNSWLGSMLWPEQYSIKVMS |
| | | ESLDKPVKITDSTPTNTIRSKEVSDSITYGIGGGVKIEGKTPGVAT |
| | | DVNAAFTKTISYHQPDYETTKVKGTISESLWNTSFTETIDGYNR |
| | | TSWNPVYGNQMFMYGRYTSNIKNNFTPDYQLSPLIRGGFSPSY |
| | | GIVMRGSKDVQKSRVKVIFGRTSETYEQSWSGFNWVGRNFLN |
| | | QSTSDWYASSTFLFEIDWQNHTVKFIA |
| 15 | Epx7 with | AIISILLASSPVFADFNESNTIGTTTTEKDEDGKTKTIITVKDRQIE |
| | signal | SYTSTDAATSSNNSSVSINATFFNDRYSDKLTTLLSFKGFIPSGR |
| | sequence | RFTFPPRNSWLGFMLWPYKYSTTITNISLDKPVKIIESTPSNTIKN |
| | | KEVSDSIVYGVGGGVKLEGKTPGASLDANAAFSKTISYQQPDY |
| | | ETIKTSGTTQKTSWDTVFTETTDGYTRTSWNSLFGNGLFMVGL |
| | | YTYDVKKNFTPDYKLSQLITGGFSPEYGLVLNAPKEVKKSQIKV |
| | | QLSRVSETYRQEWTGFNWFGRNYLDQNTSDWSSTSSLLFEIDW |
| | | EKHTVKFIA |
| 16 | Epx8 with | MLKNSRMVLVLALLANFVVGTPVLADYNEDNILGTTTTETDEN |
| | signal | GNIKTIITVKNRQIETYNSSDSATTSNNSSITVNGTFFNDKYSQEL |
| | sequence | TTILSVSGFVPSGRKFIFPPHNTWVGSMLWPVQYSTTVKNSSKD |
| | | NTVKISDSTPANTIRNKEVSNSITYGIGGGVKIEGKTPGANLDSS |
| | | VAFTKTISYQQPDYETVKTVGTTQMATWKTTFTETTDGYTRNS |
| | | WNPTYGNEMFMYGRYTYDVKKNFTPDYKLSPLITGGFSPSYGI |
| | | VLKARKNTKKSTIEVFLDRQSETYKQEWSGFNWYGSNYLDQT |
| | | KPDWSSGAALTFEVDWQNHTIRFIA |
| 17 | Exp1 K50A | QDVQVFADNPVLGTEVTFEKDENGRIVAIISKNQYRITIYNSVD |
| | | AGDTPNDASVSLDVDFIDDKNSGEMGAVASINTFIPSGLRYVEG |
| | | YKYKGVTNPIYKNLSAGMLWPKKYRVEVVNIPIDQATKIITATP |
| | | NNNIKEKQVSDTISYGFGGSVSADGKKPGGSIEANLAYTKTTTY |
| | | DQPDYETSQIKKTTKEAVWDTSFVETRDGYTPNSWNPVYGNQ |
| | | MFMRGRYSNVSPIDNIKKGGEVSSLISGGFSPKMGVVLASPNGT |
| | | KKSQFVVRVSRMSDMYIMRWSGTEWGGENEINQNVPKEYNAL |
| | | MYEDVKFEIDWEQRTVRTI |
| 18 | Exp1 K50E | QDVQVFADNPVLGTEVTFEKDENGRIVEIISKNQYRITIYNSVD |
| | | AGDTPNDASVSLDVDFIDDKNSGEMGAVASINTFIPSGLRYVEG |
| | | YKYKGVTNPIYKNLSAGMLWPKKYRVEVVNIPIDQATKIITATP |
| | | NNNIKEKQVSDTISYGFGGSVSADGKKPGGSIEANLAYTKTTTY |
| | | DQPDYETSQIKKTTKEAVWDTSFVETRDGYTPNSWNPVYGNQ |
| | | MFMRGRYSNVSPIDNIKKGGEVSSLISGGFSPKMGVVLASPNGT |
| | | KKSQFVVRVSRMSDMYIMRWSGTEWGGENEINQNVPKEYNAL |
| | | MYEDVKFEIDWEQRTVRTI |
| 19 | Exp1 | QDVQVFADNPVLGTEVTFEKDENGRIVEIISENQYRITIYNSVDA |
| | K50E/K54E | GDTPNDASVSLDVDFIDDKNSGEMGAVASINTFIPSGLRYVEGY |
| | | KYKGVTNPIYKNLSAGMLWPKKYRVEVVNIPIDQATKIITATPN |
| | | NNIKEKQVSDTISYGFGGSVSADGKKPGGSIEANLAYTKTTTYD |
| | | QPDYETSQIKKTTKEAVWDTSFVETRDGYTPNSWNPVYGNQM |
| | | FMRGRYSNVSPIDNIKKGGEVSSLISGGFSPKMGVVLASPNGTK |
| | | KSQFVVRVSRMSDMYIMRWSGTEWGGENEINQNVPKEYNAL |
| | | MYEDVKFEIDWEQRTVRTI |
| 20 | Epx2 | SFNEDNILGTTTKETDEDGKTATIITVKDRQIESYTSSDSATKEN |
| | K50A | NTSLTVNANFLNDKYSSDLTTIMSLNGFIPSGKQYYFPPNNTWV |
| | | GNMYWPSQYKTTVSSESLDQTVKIINTTPNNAIRNKDVSTSITY |
| | | GVGGGVKVEGKTVGANIDANAAFTKTISYEQPDYETVQTQWT |
| | | TDKTTWETNFTETTDGYTRTSWNTFYGNQIFMENPYTYNVKN |
| | | NFTPNYKLSPLIAGGFSPSYGVVLKAPKNTKKSIVTVYMSRIND |
| | | TYQQYWNGFNWVGRDFVDQSYSNWITNSNFKYEINWEEHTV |
| | | KFIA |
| 21 | Epx2 K50E | SFNEDNILGTTTKETDEDGKTETIITVKDRQIESYTSSDSATKEN |
| | | NTSLTVNANFLNDKYSSDLTTIMSLNGFIPSGKQYYFPPNNTWV |
| | | GNMYWPSQYKTTVSSESLDQTVKIINTTPNNAIRNKDVSTSITY |
| | | GVGGGVKVEGKTVGANIDANAAFTKTISYEQPDYETVQTQWT |
| | | TDKTTWETNFTETTDGYTRTSWNTFYGNQIFMENPYTYNVKN |
| | | NFTPNYKLSPLIAGGFSPSYGVVLKAPKNTKKSIVTVYMSRIND |
| | | TYQQYWNGFNWVGRDFVDQSYSNWITNSNFKYEINWEEHTV |
| | | KFIA |
| 22 | Epx2 | SFNEDNILGTTTKETDEDGKTETIITVEDRQIESYTSSDSATKEN |
| | K50E/K56E | NTSLTVNANFLNDKYSSDLTTIMSLNGFIPSGKQYYFPPNNTWV |
| | | GNMYWPSQYKTTVSSESLDQTVKIINTTPNNAIRNKDVSTSITY |
| | | GVGGGVKVEGKTVGANIDANAAFTKTISYEQPDYETVQTQWT |
| | | TDKTTWETNFTETTDGYTRTSWNTFYGNQIFMENPYTYNVKN |
| | | NFTPNYKLSPLIAGGFSPSYGVVLKAPKNTKKSIVTVYMSRIND |
| | | TYQQYWNGFNWVGRDFVDQSYSNWITNSNFKYEINWEEHTV |
| | | KFIA |
| 23 | Exp4 | SEDNIIGTTTQEIDEHGNVATIITVKNQQIESYTSTDSGTAKNRST |
| | K51A | LTVNANFLNDKsYSNELTTILSLNGFIPSGRKFIFPKNNTLKGEM |
| | | LWPQRYSTAVYNIPLDKSVKITNSTPDNTIRSKEVSNSITYGIGG |
| | | GIKMEGKQPGANLDANAAITKTISYQQPDYETAKTTSTVTGVN |
| | | WNTNFTETRDGYTRNSWNPVYGNQMFMYGRYTSNIRNNFTPD |
| | | YQLSSLITSGFSPSYGLVLRAPKDVKKSRIKVVFARRSETYQQN |
| | | WDGLNWWGRNFYDTKNPDSLSKVTLTFELDWQNHRVTFIE |
| 24 | Exp4 K51E | SEDNIIGTTTQEIDEHGNVETIITVKNQQIESYTSTDSGTAKNRST |
| | | LTVNANFLNDKYSNELTTILSLNGFIPSGRKFIFPKNNTLKGEML |
| | | WPQRYSTAVYNIPLDKSVKITNSTPDNTIRSKEVSNSITYGIGGG |
| | | IKMEGKQPGANLDANAAITKTISYQQPDYETAKTTSTVTGVNW |
| | | NTNFTETRDGYTRNSWNPVYGNQMFMYGRYTSNIRNNFTPDY |
| | | QLSSLITSGFSPSYGLVLRAPKDVKKSRIKVVFARRSETYQQNW |
| | | DGLNWWGRNFYDTKNPDSLSKVTLTFELDWQNHRVTFIE |
| 25 | Exp4 | SEDNIIGTTTQEIDEHGNVETIITVENQQIESYTSTDSGTAKNRST |
| | K51E/K57E | LTVNANFLNDKYSNELTTILSLNGFIPSGRKFIFPKNNTLKGEML |
| | | WPQRYSTAVYNIPLDKSVKITNSTPDNTIRSKEVSNSITYGIGGG |
| | | IKMEGKQPGANLDANAAITKTISYQQPDYETAKTTSTVTGVNW |
| | | NTNFTETRDGYTRNSWNPVYGNQMFMYGRYTSNIRNNFTPDY |
| | | QLSSLITSGESPSYGLVLRAPKDVKKSRIKVVFARRSETYQQNW |
| | | DGLNWWGRNFYDTKNPDSLSKVTLTFELDWQNHRVTFIE |
| 26 | Epx1 | MLKLKKIFLFTMISVSLCCGFG |
| | signal | |
| | sequence | |
| 27 | Epx2 | MKCMNLKKIFLFNTVIISILLVSSPVSA |
| | signal | |
| | sequence | |
| 28 | Epx3 | MKLKFALSVLSLSTILLSASPVL |
| | signal | |
| | sequence | |
| 29 | Epx4 | MIKLKKKNYKIWLSIAVLSAMLTTVPVFANF |
| | signal | |
| | sequence | |
| 30 | Epx5 | MVKLKKKNHKIWISIAVLSAMLTTVPVF |
| | signal | |
| | sequence | |
| 31 | Epx6 | MKIFRKSISKRIFFCAVLSGLMYTSPVQ |
| | signal | |
| | sequence | |
| 32 | Epx7 | AIISILLASSPVF |
| | signal | |
| | sequence | |
| 33 | Epx8 | MLKNSRMVLVLALLANFVVGTPVL |
| | signal | |
| | sequence | |

### Polynucleotides, Cells and Purification

Further provided herein are isolated and/or recombinant nucleic acids encoding any of the isolated Epx polypeptides, modified Epx polypeptides, or fusion proteins disclosed herein. The nucleic acids encoding the isolated Epx polypeptides, modified Epx polypeptides, or fusion proteins of the present disclosure, may be DNA or RNA, double-stranded or single stranded. In certain aspects, the subject nucleic acids encoding the isolated Epx polypeptides, modified Epx polypeptides, or fusion proteins are further understood to include nucleic acids encoding polypeptides that are variants of any of the isolated Epx polypeptides, modified Epx polypeptides, or fusion proteins described herein.

Variant nucleotide sequences include sequences that differ by one or more nucleotide substitutions, additions or deletions, such as allelic variants. In some embodiments, the isolated nucleic acid molecule of the present disclosure comprising a polynucleotide encoding a polypeptide comprising an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identity of any of SEQ ID NOs: 1-25. In some embodiments, the isolated nucleic acid molecule of the present disclosure comprising a polynucleotide encoding a polypeptide comprising an amino acid sequence that has 85%, 86%, 87%, 88%, 89%, 90%, 91%, 92%, 93%, 94%, 95%, 96%, 97%, 98%, 99%, or 100% identity of any of SEQ ID NOs: 1-25.

In some embodiments, the nucleic acid is comprised within a vector, such as an expression vector. In some embodiments, the vector comprises a promoter operably linked to the nucleic acid.

A variety of promoters can be used for expression of the polypeptides described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)].

Other systems include FK506 dimer, VP16 or p65 using astradiol, RU486, diphenol murislerone, or rapamycin. Inducible systems are available from Invitrogen, Clontech and Ariad. Regulatable promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the polypeptides described herein. In some embodiments, the expression of the polypeptides described herein is regulated by a constitutive, an inducible or a tissue-specific promoter. The host cells used to express the isolated polypeptides described herein may be either bacterial cells such as Escherichia coli, or, preferably, eukaryotic cells. In particular, mammalian cells, such as Chinese hamster ovary cells (CHO), in conjunction with a vector such as the major intermediate early gene promoter element from human cytomegalovirus is an effective expression system for immunoglobulins (Foecking et al. (1986) “Powerful And Versatile Enhancer-Promoter Unit For Mammalian Expression Vectors,” Gene 45:101-106; Cockett et al. (1990) “High Level Expression Of Tissue Inhibitor Of Metalloproteinases In Chinese Hamster Ovary Cells Using Glutamine Synthetase Gene Amplification,” Biotechnology 8:662-667). A variety of host-expression vector systems may be utilized to express the isolated polypeptides described herein. Such host-expression systems represent vehicles by which the coding sequences of the isolate d polypeptides described herein may be produced and subsequently purified, but also represent cells which may, when transformed or transfected with the appropriate nucleotide coding sequences, express the isolated polypeptides described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the isolated polypeptides described herein; yeast (e.g., Saccharomyces pichia) transformed with recombinant yeast expression vectors containing sequences encoding the isolated polypeptides described herein; insect cell systems infected with recombinant virus expression vectors (e.g., baclovirus) containing the sequences encoding the isolated polypeptides described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the isolated polypeptides described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the polypeptides being expressed. For example, when a large quantity of such a protein is to be produced, for the generation of pharmaceutical compositions of polypeptides described herein, vectors which direct the expression of high levels of fusion protein products that are readily purified may be desirable. Such vectors include, but are not limited, to the E. coli expression vector pUR278 (Rüther et al. (1983) “Easy Identification Of cDNA Clones,” EMBO J. 2:1791-1794), in which the coding sequence may be ligated individually into the vector in frame with the lac Z coding region so that a fusion protein is produced; pIN vectors (Inouye et al. (1985) “Up-Promoter Mutations In The lpp Gene Of Escherichia Coli,” Nucleic Acids Res. 13:3101-3110; Van Heeke et al. (1989) “Expression Of Human Asparagine Synthetase In Escherichia Coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione.

The pGEX vectors are designed to include thrombin or factor Xa protease cleavage sites so that the cloned target gene product can be released from the GST moiety. In an insect system, Autographa californica nuclear polyhedrosis virus (AcNPV) is used as a vector to express foreign genes. The virus grows in Spodoptera frugiperda cells. The coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan et al. (1984) “Adenovirus Tripartite Leader Sequence Enhances Translation Of mRNAs Late After Infection,” Proc. Natl. Acad. Sci. USA 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic.

The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544). In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

The disclosure thus encompasses engineering a nucleic acid sequence to encode a polyprotein precursor molecule comprising the polypeptides described herein, which includes coding sequences capable of forming pores and/or causing cellular toxicity.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T. 3T3, WI38, BT483, Hs578T, HTB2, BT20 and T47D, CRL7030 and Hs578Bst.

For long-term, high-yield production of recombinant proteins, stable expression is preferred. For example, cell lines which stably express polypeptides described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptides described herein. Such engineered cell lines may be particularly useful in screening and evaluation of polypeptides that interact directly or indirectly with the polypeptides described herein.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11:223-232), hypoxanthine-guanine phosphoribosyltransferase (Szybalska et al. (1992) “Use Of The HPRT Gene And The HAT Selection Technique In DNA-Mediated Transformation Of Mammalian Cells First Steps Toward Developing Hybridoma Techniques And Gene Therapy,” Bioessays 14:495-500), and adenine phosphoribosyltransferase (Lowy et al. (1980) “Isolation Of Transforming DNA: Cloning The Hamster aprt Gene,” Cell 22:817-823) genes can be employed in tk-, hgprt- or aprt-cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. USA 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. USA 78:1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. USA 78:2072-2076); neo, which confers resistance to the aminoglycoside G-418 (Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy.” Science 260:926-932; and Morgan et al. (1993) “Human Gene Therapy,” Ann. Rev. Biochem. 62:191-217) and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY; Colberre-Garapin et al. (1981) “A New Dominant Hybrid Selective Marker For Higher Eukaryotic Cells,” J. Mol. Biol. 150:1-14.

The expression levels of polypeptides described herein can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing a polypeptide described herein is amplifiable, increase in the level of inhibitor present in culture of host cell will increase the number of copies of the marker gene. Since the amplified region is associated with the nucleotide sequence of a polypeptide described herein or a polypeptide described herein, production of the polypeptide will also increase (Crouse et al. (1983) “Expression And Amplification Of Engineered Mouse Dihydrofolate Reductase Minigenes,” Mol. Cell. Biol. 3:257-266).

Once a polypeptide described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the polypeptide comprises an Fc domain (or portion thereof)), and sizing column chromatography), centrifugation, differential solubility, or by any other standard technique for the purification of polypeptides or antibodies. Other aspects of the present disclosure relate to a cell comprising a nucleic acid described herein or a vector described herein.

The cell may be a prokaryotic or eukaryotic cell. In some embodiments, the cell in a mammalian cell. Exemplary cell types are described herein. Other aspects of the present disclosure related to a cell expressing the modified isolated Epx polypeptides, modified Epx polypeptides, or fusion proteins described herein. The cell may be a prokaryotic or eukaryotic cell. In some embodiments, the cell in a mammalian cell. Exemplary cell types are described herein. The cell can be for propagation of the nucleic acid or for expression of the nucleic acid, or both. Such cells include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human. Cell lines may be obtained from the American Type Culture Collection, European Collection of Cell Cultures and the German Collection of Microorganisms and Cell Cultures. Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rac, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4.sup.th ed. 2000); ANIMAL CELL CULTURE: A PRACTICAL APPROACH (John R. W. Masters ed., Oxford University Press, 3.sup.rd ed. 2000); MOLECULAR CLONING A LABORATORY MANUAL, supra, (2001); BASIC CELL CULTURE: A PRACTICAL APPROACH (John M. Davis, Oxford Press, 2.sup.nd ed. 2002); and CURRENT PROTOCOLS IN MOLECULAR BIOLOGY, supra, (2004).

These protocols are routine procedures within the scope of one skilled in the art and from the teaching herein. Yet other aspects of the present disclosure relate to a method of producing a polypeptide described herein, the method comprising obtaining a cell described herein and expressing nucleic acid described herein in said cell. In some embodiments, the method further comprises isolating and purifying a polypeptide described herein.

In some embodiments, Epx polypeptides can be obtained by establishing and growing cultures of Enterococci in a fermenter and then harvesting and purifying the fermented mixture in accordance with known procedures.

### Methods

In some aspects, the present application discloses methods of inducing an immune response comprising administering to a subject the modified Epx polypeptides or compositions disclosed herein.

An “immune response” may refer to any response by the immune system including, but not limited to an innate immune response (e.g., inflammation, fever, cough, mucus production, and cytokine production), an adaptive immune response (e.g., immunoglobin production/secretion and T cell activation).

As used herein, a “subject” refers to a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human.

The terms, “patient” and “subject” are used interchangeably herein. A subject can be male or female. A subject can be a fully developed subject (e.g., an adult) or a subject undergoing the developmental process (e.g., a child, infant or fetus). Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with unwanted neuronal activity. In addition, the methods and compositions described herein can be used to treat domesticated animals and/or pets.

A subject may refer to an individual who has a disease, a symptom of the disease, a predisposition toward the disease, or is need of protection from a disease (e.g. in need of vaccination). In some embodiments, the subject has, has a predisposition for, or is in need of protection from a viral, bacterial, fungal or parasitic disease as described herein. In some embodiments, the subject has, has a predisposition for, or is in need of protection from cancer as described herein. In some embodiments, the subject has, has a predisposition for, or is in need of protection from a disease associated with expression of an Epx polypeptide (e.g., a bacterial disease where the bacteria expresses an Epx polypeptide). In some embodiments, the subject has a disease associated with MHC class I expression or activation. In some embodiments, the subject has a disease selected from the group consisting of idiopathic inflammatory muscle diseases, diabetes, chronic inflammation, rheumatoid Arthritis, ankylosing spondylitis, asthma, Alzheimer's disease, Inflammatory bowel disease, obesity, Fatty liver disease and Endometriosis. In some embodiments, treated of a disease includes delaying the development or progression of the disease, or reducing disease severity. In some embodiments, treatments of the disease does not necessarily require curative results.

Conventional methods, known to those of ordinary skill in the art of medicine, can be used to administer the Epx polypeptide or composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

In some embodiments, the present application discloses methods of inducing an immune response against Enterococci in a subject, the method comprising administering to the subject a modified Exp polypeptide or a composition as described herein. In some embodiments, the Enterococci is a multi-drug resistant Enterococci (e.g. multidrug resistant Enterococcus faecalis).

In some embodiments, the present application discloses methods of inducing an immune response against an antigen in a subject, the method comprising administering to the subject the composition as described herein (e.g. comprising a modified Epx polypeptide or fragment thereof, and an antigen). In some embodiments, the antigen may be any antigen described herein. In some embodiments, the method is therapeutic (e.g. to treat a disease). In some embodiments, the method is prophylactic (e.g. to prevent a disease). In some embodiments, the antigen is associated with any disease described herein.

In some aspects, the present application discloses that the Epx polypeptide may bind to major histocompatibility complex (MHC) class I receptors (also called the Human leukocyte antigen (HLA) complex in humans). In some embodiments, the present application discloses methods of binding MHC class I using an Epx polypeptide. In some embodiments, the present application discloses methods of binding MHC class I using an Epx polypeptide (e.g. Exp2 or Exp3). In some embodiments, the Epx polypeptide binds to at least a portion of the beta-2-microglobulin (B2M) subunit of MHC class I. In some embodiments, the Epx polypeptide only binds to a MHC class I complex when the MHC class I complex comprises B2M. In some embodiments, the Epx polypeptide only binds to a MHC class I/HLA-A complex when the MHC class I complex comprises B2M. In some embodiments, Epx2 and Epx3 polypeptide only bind to the MHC class I/HLA-A complex when the MHC class I complex comprises B2M. In some embodiments, the Epx polypeptide binds to a α1-α2 region of the MHC class I α-subunit. In some embodiments, the MHC class I receptor is equine, bovine, and porcine. In some embodiments, the MHC class I receptor is not murine.

In some embodiments, the present application discloses methods of blocking MHC class I activity, the method comprising contacting an MHC class I receptor with the isolated Epx polypeptide described herein (e.g. Epx2 or Epx3), the modified Epx polypeptide described herein, or the composition described herein. In some embodiments, the present application discloses methods of blocking MHC class I activity, the method comprising contacting an MHC class I receptor Epx2 or Epx3 or a variant thereof.

In some embodiments, blocking MHC class I activity may include inhibiting MHC class I from presenting an antigen. In some embodiments, blocking MHC class I activity may include disrupting the immune system from recognizing a presented antigen. In some embodiments, blocking MHC class I activity may include disrupting the immune system from destroying a cell that is presenting an antigen. In some embodiments, blocking MHC class I activity comprises blocking at least 10% (e.g. at least 10%, at least 25%, at least 50%, at least 75%, at least 85%, at least 90%, at least 95%, or at least 99%) of the activity of the MHC class I receptor.

In some embodiments, the contacting occurs in an cell free assay (e.g. a binding assay). In some embodiments, the contacting occurs in in vitro cell culture (e.g. mammalian cell culture, cancer cell culture, or a mixed cell culture comprising MHC Class I expressing cells and immune cells (e.g. T-cells)). In some embodiments, the contacting occurs in a subject (e.g. a human or an animal subject).

In some embodiments, the present application discloses methods of treating a disease associated with detrimental MHC class I activity, the method comprising administering to a subject a modified Epx polypeptide described herein, or a composition described herein. In some embodiments, the subject has a disease is selected from the group consisting of cancer, an autoimmune disease, a bacterial infection, a viral infection, a parasitic infection, or a fungal infection. In some embodiments, the subject has an infection from any of the viruses, bacterial, fungi, or parasites described herein. In some embodiments, the subject has cancer. In some embodiments, the subject has a disease selected from the group consisting of idiopathic inflammatory muscle diseases, diabetes, chronic inflammation, rheumatoid Arthritis, ankylosing spondylitis, asthma, Alzheimer's disease, Inflammatory bowel disease, obesity, Fatty liver disease and Endometriosis.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising.” “including,” “carrying.” “having.” “containing.” “involving.” “holding.” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately.” “substantially.” and “about” may include the target value.

Each reference (e.g., patent, patent application, and non-patent literature) cited herein is incorporated by reference in its entirety.

### EXAMPLES

### Summary

Enterococci are a part of human microbiota and a leading cause of multidrug resistant infections. A family of Enterococcus pore-forming toxins (Epx) were identified in E. faecalis, E. faecium, and E. hirae strains isolated across the globe. Structural studies revealed that Epxs formed a branch of β-barrel pore-forming toxins with a β-barrel protrusion (designated the top domain) sitting atop the cap domain. Through a genome-wide CRISPR-Cas9 screen, a human leukocyte antigen—I complex (HLA-I) was identified as a receptor for two members (Epx2 and Epx3), which preferentially recognized human HLA-I and homologous MHC-I of equine, bovine, and porcine, but not murine origin. Interferon exposure, which stimulates MHC-I expression, sensitizes human cells and intestinal organoids to Epx2 and Epx3 toxicity. Co-culture with Epx2-harboring E. faecium damages human peripheral blood mononuclear cells and intestinal organoids, and this toxicity is neutralized by an Epx2 antibody, demonstrating the toxin-mediated virulence of Epx-carrying Enterococcus.

### Results

### Toxin Discovery and Genome Analysis

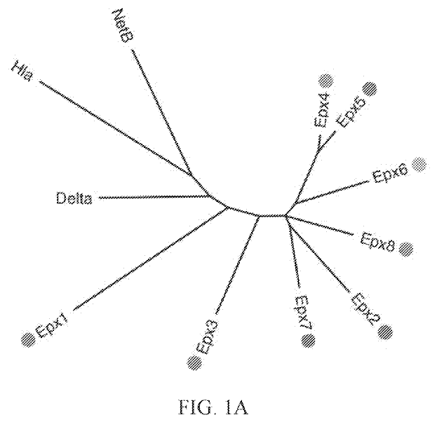

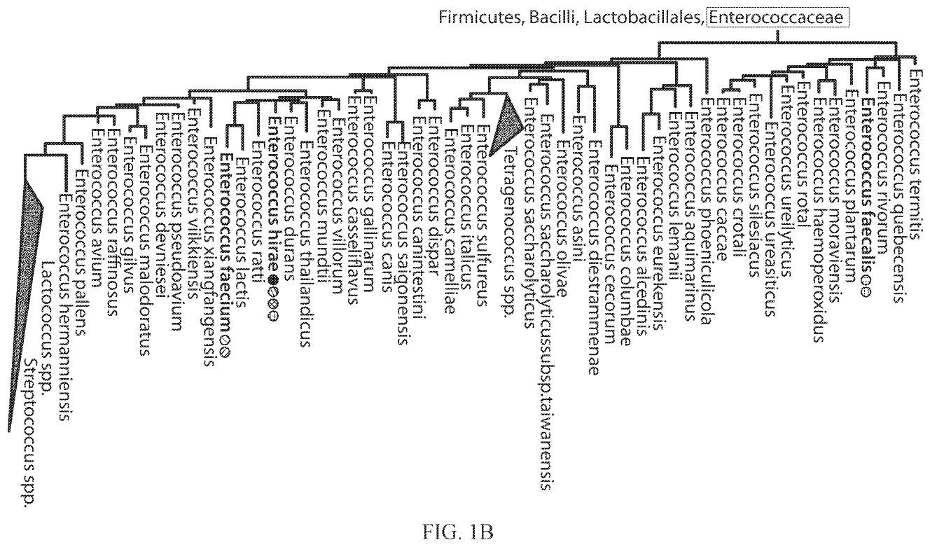

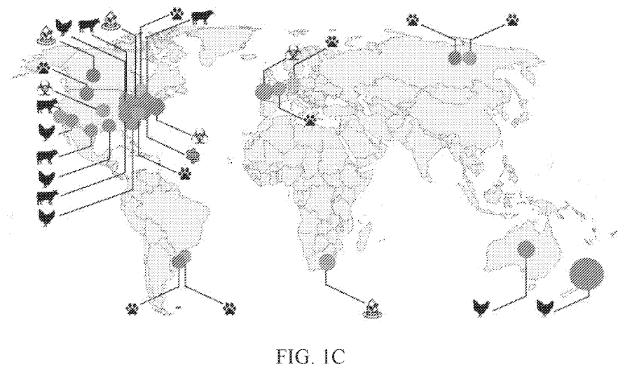

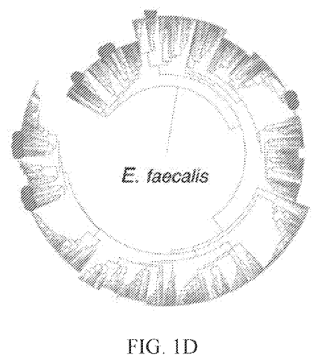

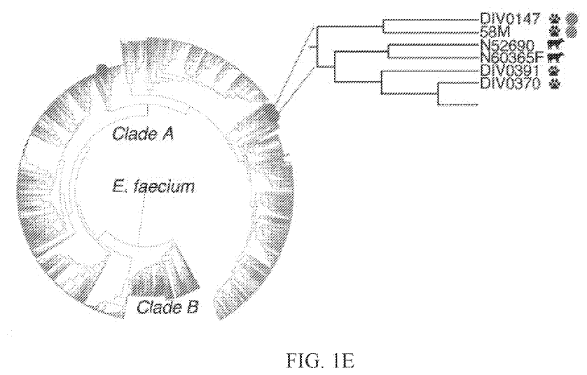

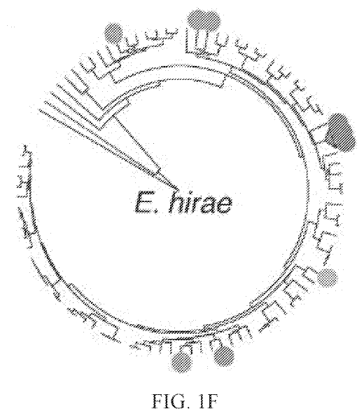

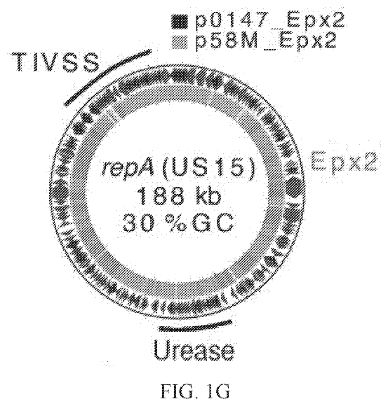

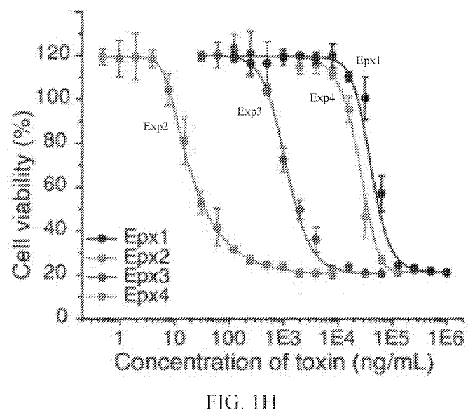

Enterococcal strains were collected and sequenced since 2011 to better understand how the natural reservoir of Enterococcus traits predisposes them to emerge as MDR hospital pathogens. A potential small β-barrel PFT gene was observed in an E. faecalis strain from chicken meat from North Carolina, and another in an E. faecium strain from horse feces collected in Montana, which was termed Enterococcus pore-forming toxin 1 and 2 (Epx1 and Epx2), respectively. The closest homolog is C. perfringens delta toxin (42% and 43% identity to Epx 1 and Epx2, respectively). Searching public databases revealed two more toxins (Epx3 and Epx4) and the most recent search identified four additional homologs (Epx5-8) (Goncharov et al., 2016; Manson et al., 2019; Poyet et al., 2019; Rushton-Green et al., 2019; Tyson et al., 2018; Weigand et al., 2014; Zaheer et al., 2020). These Epxs are 40-89% identical to each other and form a separate branch from other PFTs (FIG. 1A and Table 2).

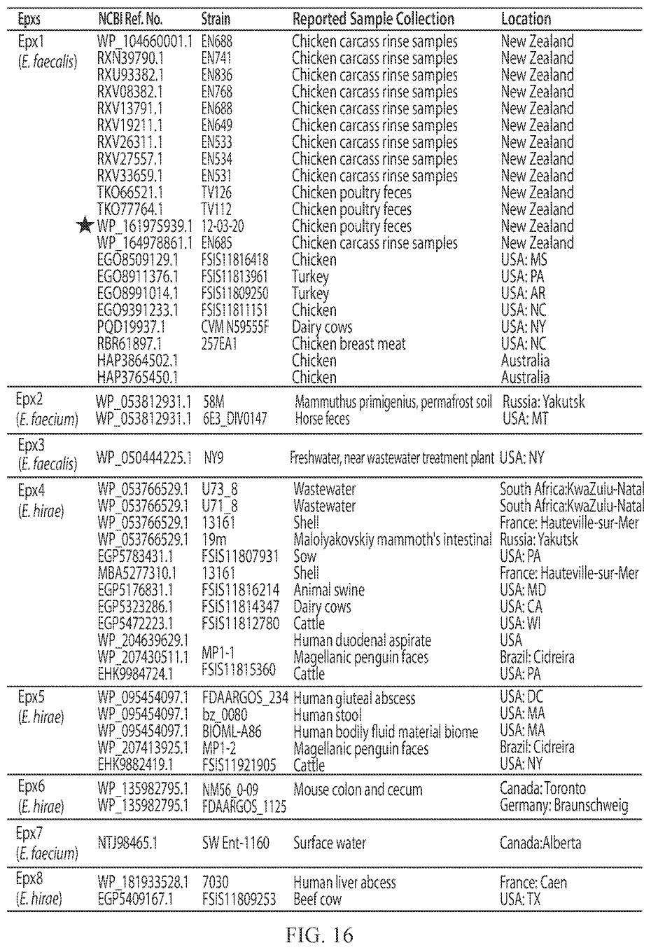

Epxs have been identified in three Enterococcus species: Epx1 and 3 in E. faecalis, Epx 2 and 7 in E. faecium, and Epx4, 5, 6, 8 in E. hirae (FIG. 1B and FIG. 16). These strains were collected across five continents and from diverse sources such as chicken meat, turkey, dairy cows, horse feces, mouse colon, 40,000-year-old mammoth intestine, wastewater, as well as human samples (stool, bodily fluid, a gluteal abscess in the United States, and a liver abscess in France) (FIG. 1C and Table 2).

TABLE 2
Protein sequence identity among Epxs and Hla family PFTs, related to FIGS. 1A-1J.

| | Epx1 | Epx2 | Epx3 | Epx4 | Epx5 | Epx6 | Epx7 | Epx8 | Delta | Hla | NetB | LukF | Hlg2 |
|---|---|---|---|---|---|---|---|---|---|---|---|---|---|
| Epx1 | — | | | | | | | | | | | | |
| Epx2 | 40.98 | — | | | | | | | | | | | |
| Epx3 | 40.87 | 53.35 | — | | | | | | | | | | |
| Epx4 | 41.77 | 57.49 | 55.18 | — | | | | | | | | | |
| Epx5 | 39.63 | 56.89 | 52.44 | 88.66 | — | | | | | | | | |
| Epx6 | 40.55 | 59.88 | 52.13 | 65.67 | 64.48 | — | | | | | | | |
| Epx7 | 42.72 | 65.31 | 56.43 | 61.87 | 61.25 | 63.75 | — | | | | | | |
| Epx8 | 41.05 | 61.33 | 57.32 | 63.75 | 62.84 | 63.14 | 67.19 | — | | | | | |
| Delta | 42.49 | 43.08 | 49.2 | 50.63 | 48.43 | 44.03 | 47.7 | 48.89 | — | | | | |
| Hla | 28.25 | 26.54 | 28.06 | 27.18 | 28.16 | 25.81 | 27.91 | 24.6 | 27.91 | — | | | |
| NetB | 33.02 | 35 | 37.9 | 36.76 | 35.2 | 35.2 | 36.6 | 36.59 | 41.67 | 25.9 | — | | |
| LukF | 26.89 | 26.47 | 30.07 | 28.01 | 28.66 | 28.9 | 29.63 | 26.89 | 28.76 | 29.26 | 24.75 | — | |
| Hlg2 | 20.62 | 24.23 | 23.63 | 27.21 | 24.83 | 23.73 | 22.65 | 21.23 | 26.32 | 23.99 | 23.79 | 30.87 | — |

Pairwise comparisons among the indicated toxin protein sequences were analyzed with Clustal Omega (ebi.ac.uk/Tools/msa/clustalo/).

Comparing the genomes of these isolates with available reference genomes (WGS RefSeq) of E. faecalis (n=1743, FIG. 1D), E. faecium (n=2197, FIG. 1E), and E. hirae (n=190, FIG. 1F) reveals that Epx-carrying isolates are not monophyletic, indicating multiple acquisition events. Notably, an Epx1-carrying E. faecalis isolate, belonging to a highly-persistent ST-108 lineage of poultry and human isolates, carries the vancomycin-resistance gene (FIG. 1D and FIG. 16). Furthermore, Epx2-carrying E. faecium strains are closely related to known lineages of hospital-adapted MDR isolates of E. faecium clade A (FIG. 1E) (Lebreton et al., 2013).

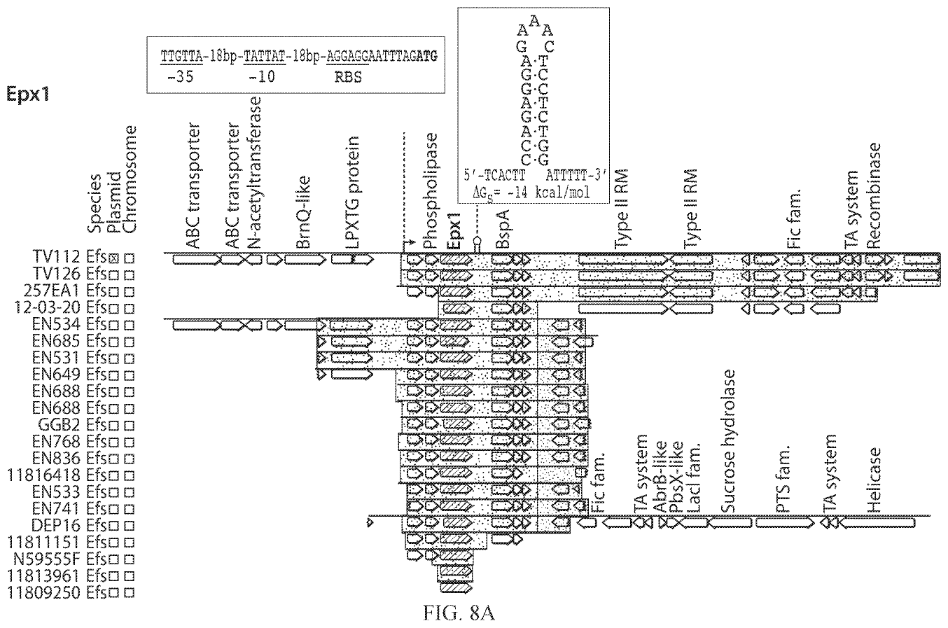

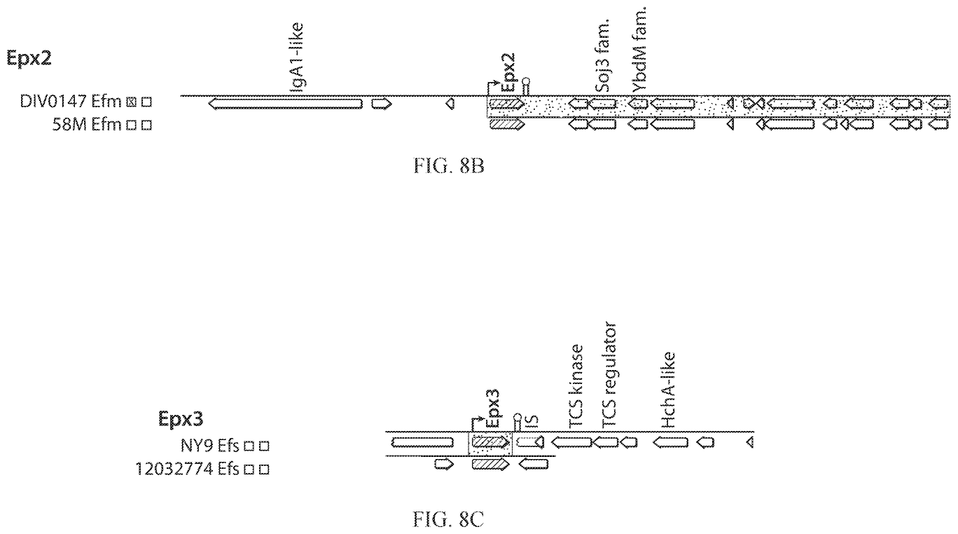

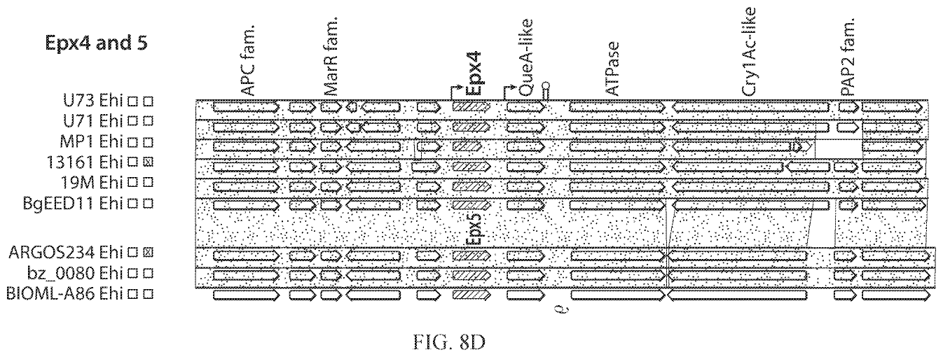

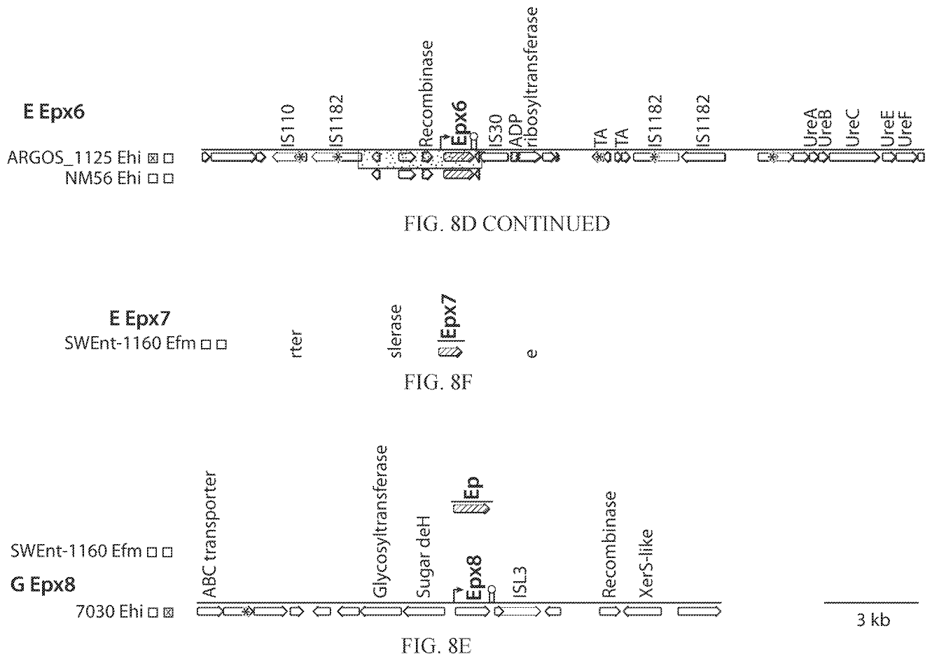

Further analysis of the DNA surrounding the epx genes revealed no overall conservation between different Epx types, except for Epx4 and 5 within E. hirae (FIGS. 8A-8F). However, within each Epx type, the genetic organization was conserved. No common auxiliary regulatory or secretion systems were found (FIGS. 8A-8F). Analysis of isolates with complete genomes (n=6) suggested that epx1, -2 and -6 are conveyed by large conjugative plasmids in three strains of E. faecalis, E. faecium and E. hirae, respectively, whereas epx4, -5, and -8 are localized within the chromosomes of E. hirae strains FIGS. 8A-8F.

Long-read sequencing of the Epx2-harboring E. faecium strain DIV0147 (isolated in the United States) confirmed that epx2 is in a repUS15 family plasmid (named p0147_Epx2, FIG. 1G). An identical Epx2 gene was found in another E. faecium isolate, 58 M, from Siberia (Goncharov et al., 2016), which shared 98.9% nucleotide identity with DIV0147. The Epx2-carrying plasmids in 58 M and DIV0147 are nearly identical (>97% nucleotide identity), suggesting that both strains vertically inherited the Epx2-plasmid. The plasmid backbone includes genes encoding a type IV secretion system and pilus assembly likely associated with its conjugative ability. These plasmids showed lower GC content (˜30%) than the average Enterococcus chromosome (37-38%).

### Epxs are Cytotoxic PFTs

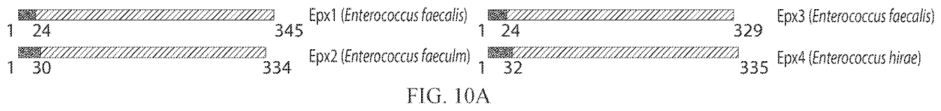

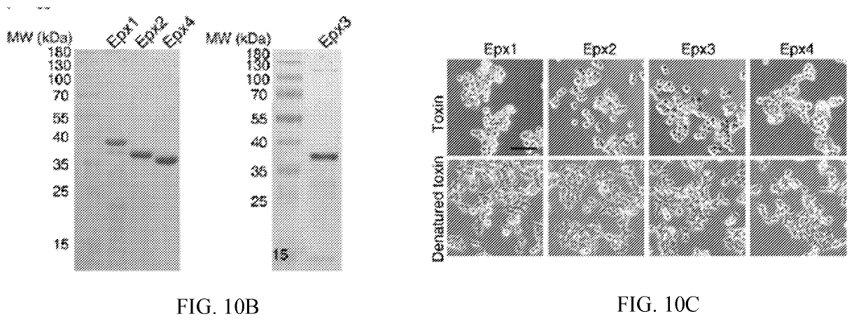

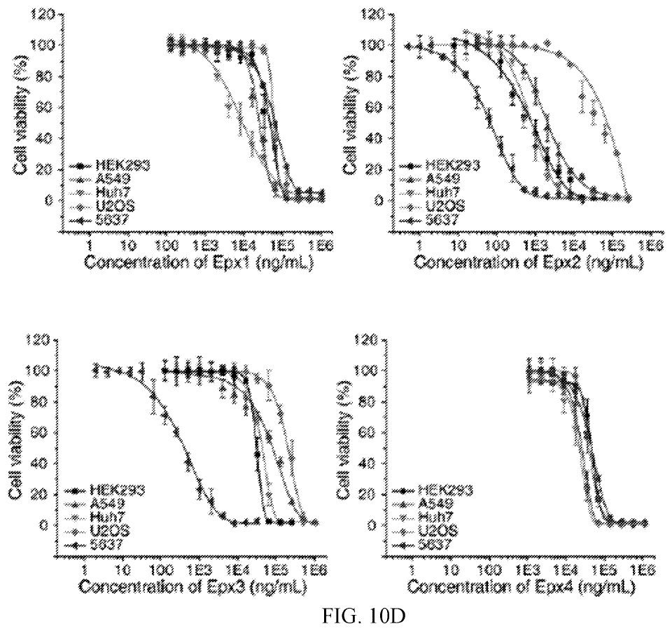

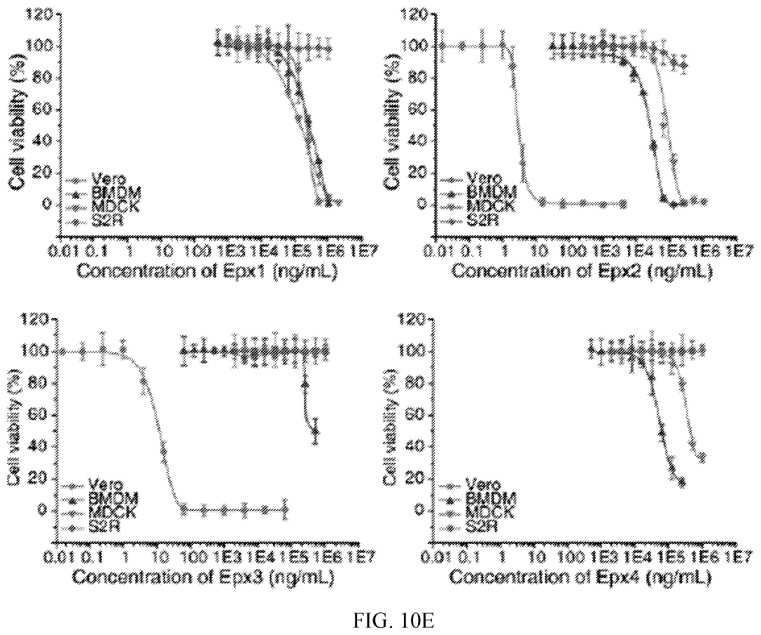

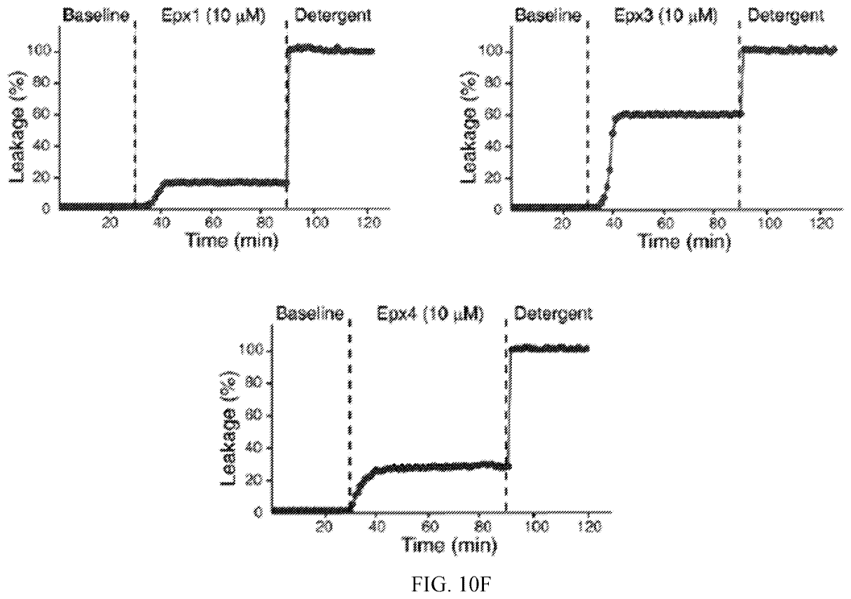

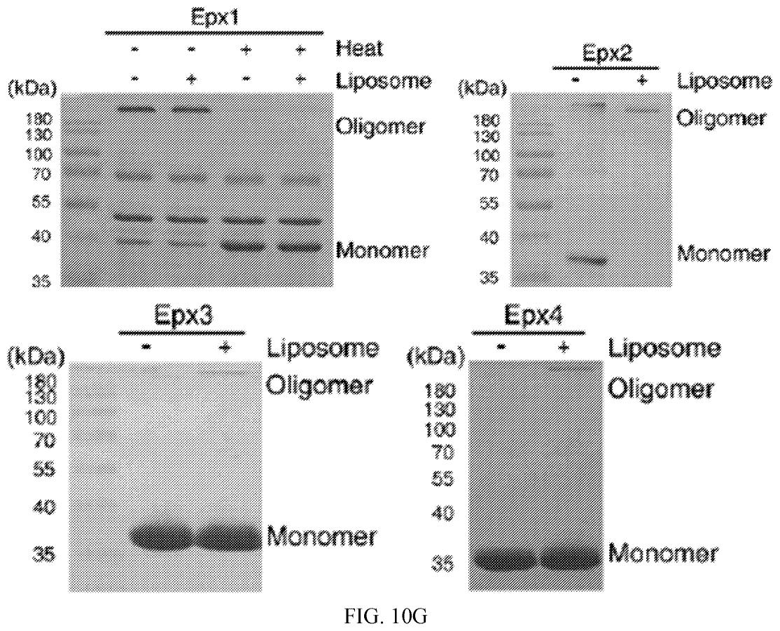

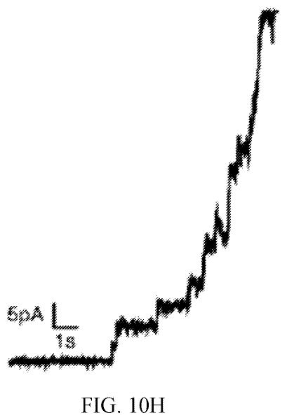

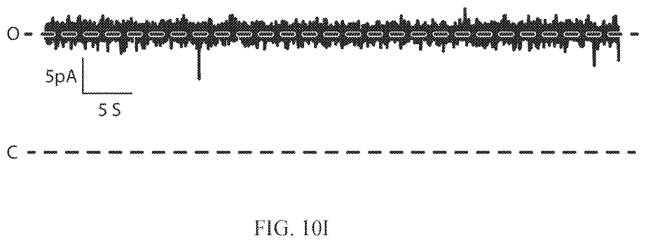

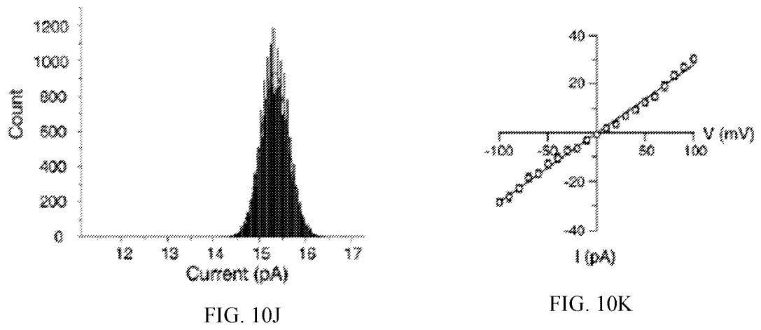

To validate the function of Epxs, Epx1-4 was produced in E. coli (FIGS. 9, 10A, and 10B). All four Epxs at a concentration of 100 μg/mL induced death of HEK cells (FIG. 10C). The susceptibility was then assessed with a range of human cell lines (HEK, HeLa, A549, Huh7, U2OS, and 5637, FIGS. 1H and 10D), and cell lines from other species (Vero cells, an immortalized mouse bone marrow-derived macrophage cell line BMDM, MDCK cells, Drosophila S2 cells, FIG. 10E), by exposing cells to dilutions of toxins and measuring cell viability with the MTT assay.

Epx2 is one of the most potent PFTs known for HeLa cells, with the dose resulting in loss of viability of 50% cells (IC₅₀) at ˜11-14 ng/ml, which is ˜100-fold more toxic than Epx3, and ˜3,000-fold more toxic than Epx1 and Epx4 (FIG. 1H). Epx1 and Epx4 showed IC₅₀>25 μg/mL on all cell lines (FIG. 10D), whereas Epx2 and Epx3 showed variable toxicity for different cell lines (FIG. 10D). Vero cells are highly sensitive to Epx2 and Epx3, whereas the toxicities on BMDM, MDCK, and S2 cells are low for all four Epxs (FIG. 10E).

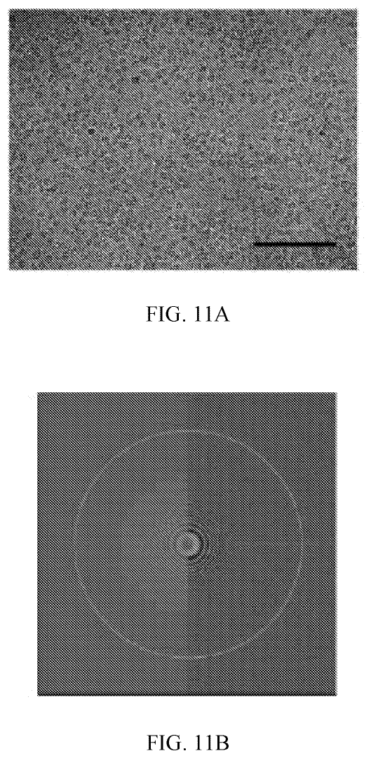

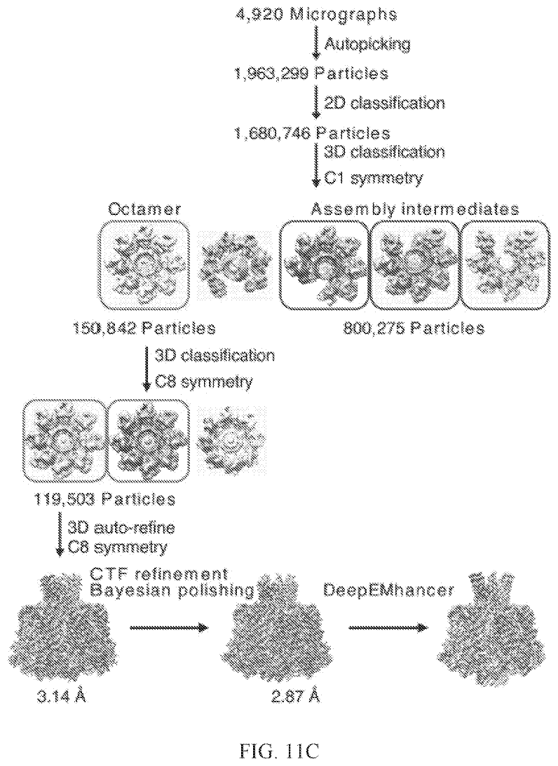

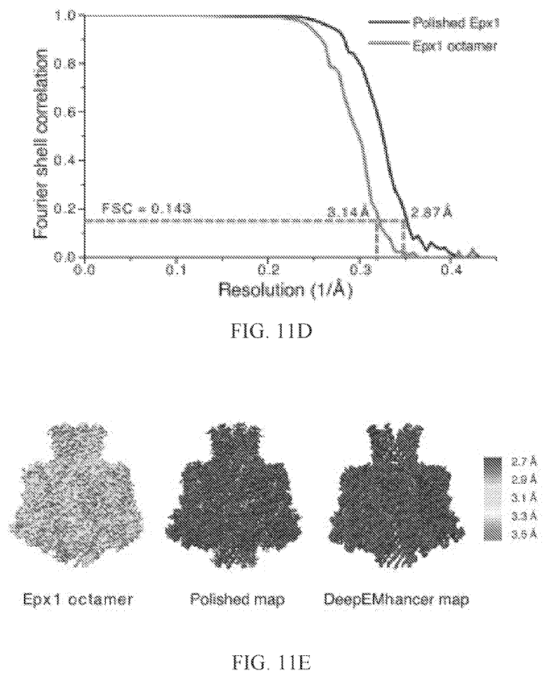

All four Epxs were capable of inducing lysis of artificial liposomes in vitro (FIGS. 11 and 10F). The presence of liposomes promoted formation of sodium dodecyl sulfate (SDS)-resistant oligomers similar to other PFTs (FIG. 10G), and Epx2 pores were observed on liposomes by negative-staining electron microscopy (EM, FIG. 1J).

### Crystal Structure of an Epx4 Octameric Pore

Crystallization screens were performed and the crystals of Epx4 were obtained, which diffracted to 3.0 Å resolution (FIGS. 2A, 2B and 16). The structure unexpectedly revealed an assembled octameric pore instead of a monomer (FIG. 2A). This was likely due to the presence of 40% 2-methyl-2,4-pentanediol (MPD) in the crystallization buffer, which has been shown to induce pore formation of known pore forming toxins Hla, γ-hemolysin, and leucocidin during crystallization (Tanaka et al., 2011; Yamashita et al., 2014; Yamashita et al., 2011). The overall architecture of the Epx4 pore resembled that of Hla, comprising of a cap domain formed by the core β-sandwich region (FIGS. 2A and 2B), a rim domain with patches of aromatic residues that likely contact cell membranes (FIG. 2C), and a stem domain forming a transmembrane β-barrel pore, with an estimated diameter of ˜18 Å (FIGS. 2A and 2B). The cap, rim, and stem domains of the Epx4 protomer were structurally similar to the protomers of Hla (PDB: 7AHL) (Song et al., 1996), with the root mean square deviations (RMSD) at 1.415 Å (FIGS. 2D and 2E). However, the octameric configuration of Epx4 pore has not been reported for a single component β-barrel PFT.

TABLE 4
Crystal structure data collection and refinement statistics,
related to FIGs. 2A-2F

| Crystal | Epx4 |
|---|---|
| Data Collection | |
| Wavelength (Å) | 0.9792 |
| Space group | I4 |
| Unit Cell | |
| a, b, c (Å) | 122.3, 122.3, 128.4 |
| α, β, γ (°) | 90, 90, 90 |
| Resolution (Å) | 64-3.0 (3.107-3.0) ^{a} |
| R_{merge} | 0.204 (0.927) |
| I/σ (I) | 8.94 (2.08) |
| Completeness (%) | 99.97 (100.00) |
| Redundancy | 6.9 (7.1) |
| Refinement (F > 0) | |
| Resolution (Å) | 64-3.0 |
| No. of reflections | 18919 |
| R_{work}/R_{free} (%) ^{b} | 20.19/24.39 |
| No. of non-H atoms | |
| Protein | 4818 |
| Lignad | 16 |
| Average B-factors (Å²) | |
| Protein | 50.10 |
| Ligand | 61.00 |
| RMSD bonds (Å) | 0.002 |
| RMSD angle (°) | 0.66 |

^{a} Numbers in parentheses refer to the highest resolution shells.
^{b} R = Σₕ∥F_{obs}| − |F_{cal}∥/Σₕ|F_{obs}|, where F_{obs} and F_{cal} are the observed and calculated structure factors, respectively. R_{work} and R_{free} were calculated by using the working and test set reflections, respectively.

An unexpected feature of the Epx4 pore was the formation of a second β-barrel that sits on top of the cap region (FIGS. 2A-2E). It was termed the “top domain”, which was formed by a β-hairpin located at the N-terminus of each protomer (residues 34-61, FIGS. 2D and 9). The diameter of this top domain is similar to that of the transmembrane β-barrel pore. The top domain, the cap region, and the stem β-barrel pore form a continuous channel, with the top domain extending the channel by 28 Å (FIGS. 2E and 2F).

### Cryo-EM Structure of Epx1 Prepore

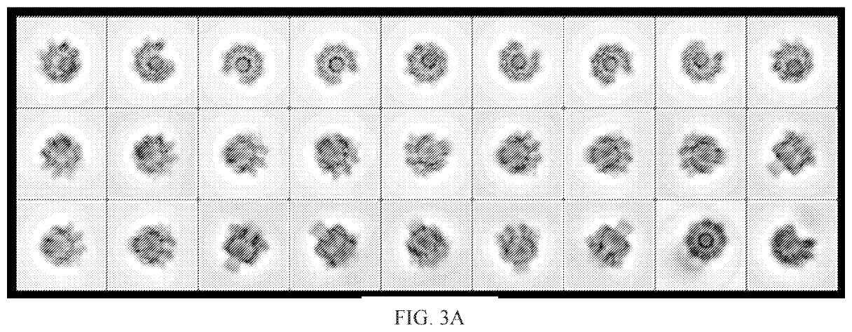

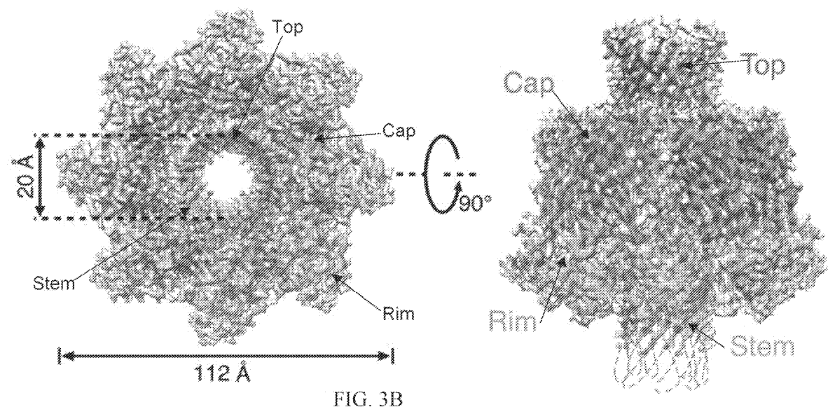

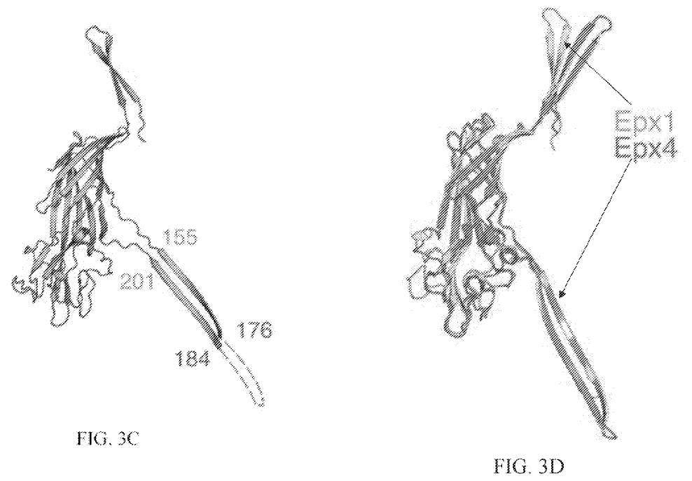

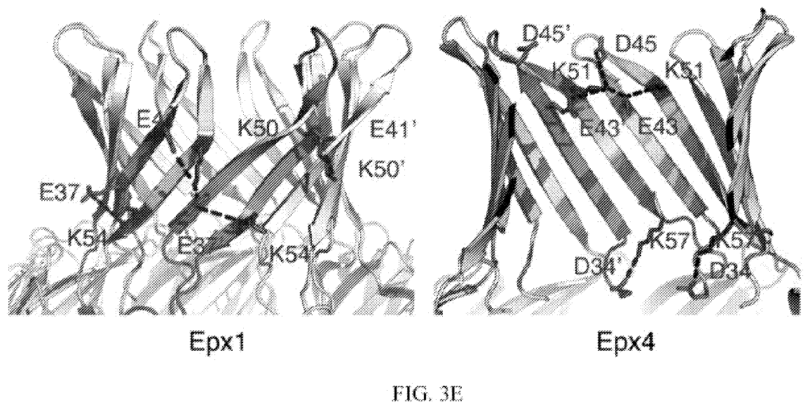

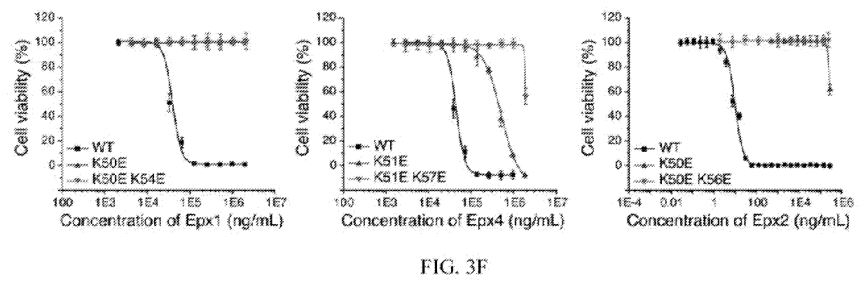

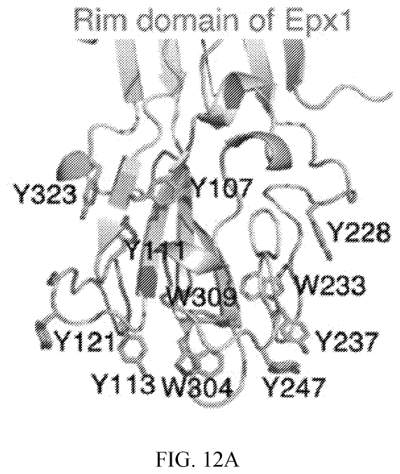

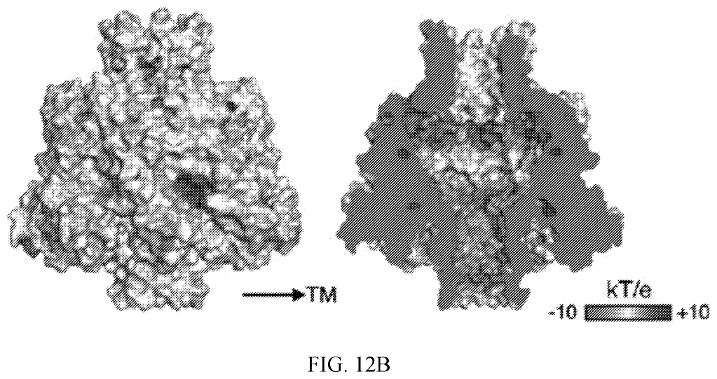

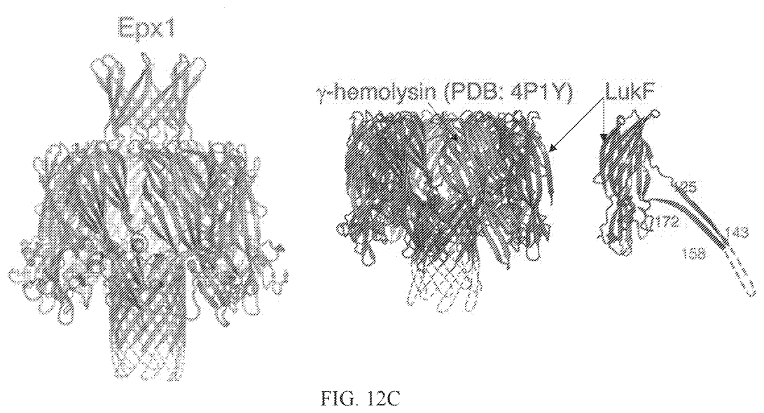

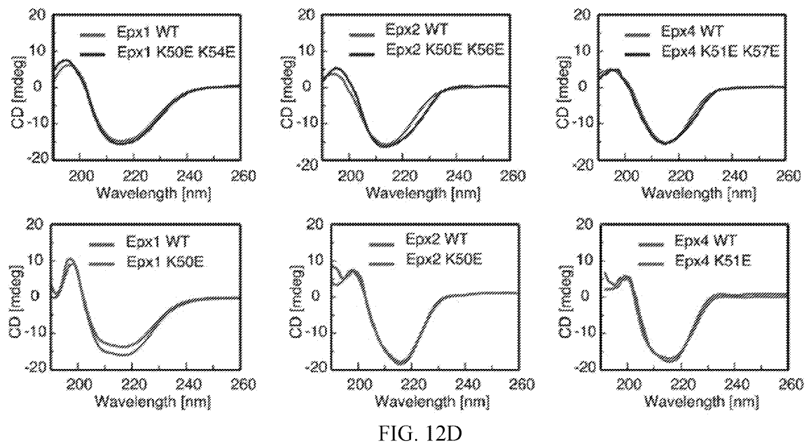

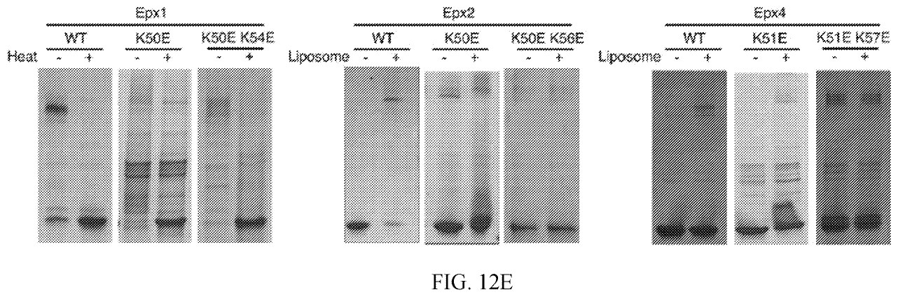

Among Epxs, it was observed that Epx1 spontaneously formed SDS-resistant oligomers in solution (FIG. 10G). We took advantage of this feature and single particle cryo-EM analysis of soluble Epx1 oligomers (FIGS. 3A and 11A-11E) was conducted, which revealed assembled octameric pores at 2.9 Å as well as intermediate assemblies (FIGS. 3A and 11C; Table 4). The overall architecture was similar to that of the Epx4 pore (FIGS. 3B-3C and 12A-12B), with an RMSD of 0.83 Å (FIG. 3D), except that the bottom one third of the β-barrel pore of Epx1 was not visible in cryo-EM maps (residues 176-184, FIGS. 3B and 3C). This was reminiscent to the prepore state for γ-hemolysin and leucocidin toxins in solution (Yamashita et al., 2014), in which a pore-like configuration is assembled but the last one third of the β-barrel remains disordered (FIG. 12C).

TABLE 5
Cryo-EM data collection and refinement statistics,
related to FIGs. 3A-3F

| Cryo-EM | |
|---|---|
| Data Collection | |
| Micrographs | 4920 |
| Particles selected | 1,963,299 |
| Particles included in final reconstruction | 119,503 |
| Sampling, Å per pixel | 0.825 |
| Defocus range (μm) | −1.5~−2.5 |
| Resolution (Å) (FSC = 0.143 criterion) | 2.87 |
| Refinement (F > 0) | |
| Clashscore | 10.78 |
| MolProbity Score | 1.71 |
| Rotamer outliers (%) | 0.00 |
| C-beta deviations | 0.00 |
| Ramachandran statistics (%) | |
| Most favored | 97.1 |
| Allowed | 2.9 |
| Outliers | 0.0 |
| RMSD deviations | |
| RMSD bonds (Å) | 0.002 |
| RMSD angle (°) | 0.525 |

To further confirm that Epx1 forms functional pores, the conductance properties of Epx 1 were analyzed on planar lipid bilayers through single-channel electrophysiological recordings. Multiple stepwise pore-forming events were observed (FIGS. 10H-10J). The conductance of the pore varied linearly with the applied potential and did not show rectification (FIG. 10K). The conductance (290±20 pS) in combination with the estimated length of the pore based on the structure suggests a pore diameter of ˜2.1±0.1 nm.

### The Top Domain is Crucial for Toxicity

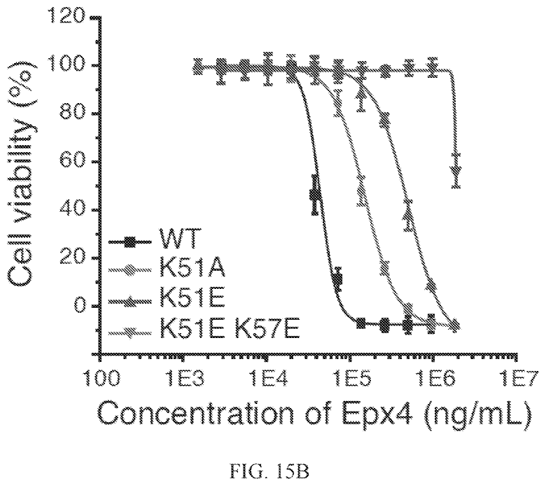

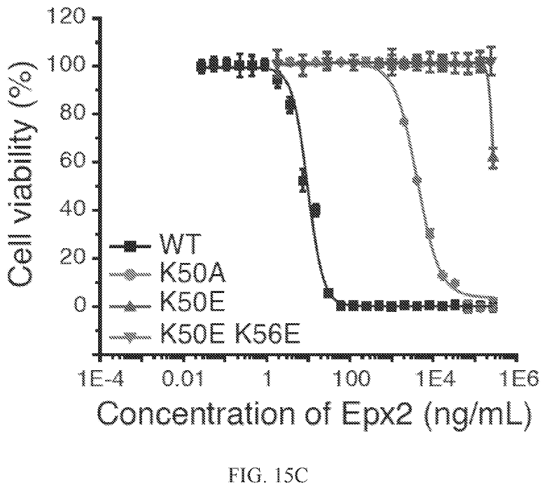

The structures of Epx1 and Epx4 revealed extensive inter-protomer interactions mediated by charged residues within their top domains (FIG. 3E). Mutations at two charged residues (K50E, K50E/K54E in Epx1, and K51E, K51E/K57E in Epx4) in the top domain, which does not alter the overall conformation of Epx proteins measured by circular dichroism spectroscopy (FIG. 12D), reduced the efficacy of forming SDS-resistant oligomers (FIG. 12E) and toxicity on HeLa cells (FIG. 3F). Similarly, mutating analogous residues (K50E, and K50E/K56E) in Epx2 disrupted formation of SDS-resistant oligomers (FIGS. 12D and 12E) and abolished toxicity on HeLa cells (FIG. 3F). Also see FIGS. 15A-15C.

Structure-based sequence alignments suggested that the top domain of Epxs corresponded to the N-terminal latch domain in other Hla family members (FIG. 9), which has been suggested to play a role in inter-protomer interactions (Huyet et al., 2013; Sugawara et al., 2015). Epx members share a conserved overall length and number of charged residues within this region, whereas Hla has a large gap within this region, missing 14 of the 30 residues (FIG. 9).

### CRISPR-Cas9 Screen Identifies MHC/HLA-I as a Receptor for Epx2

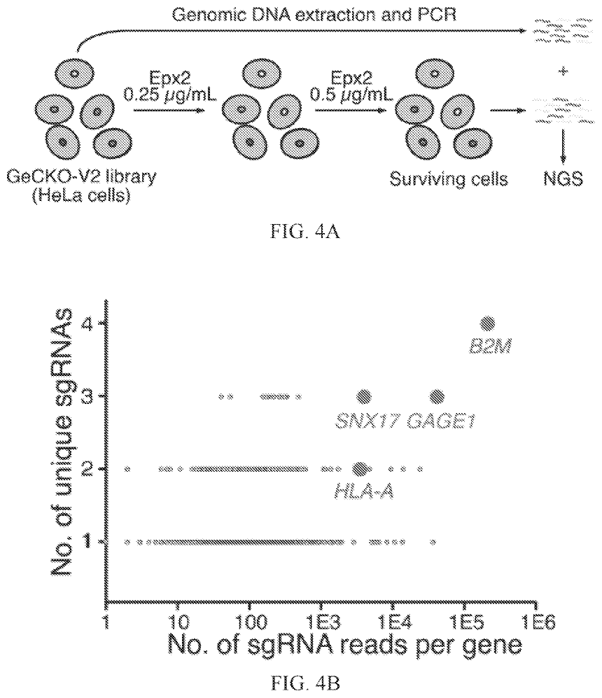

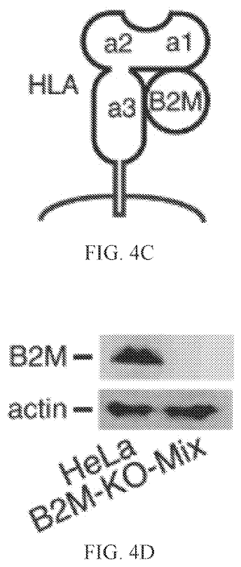

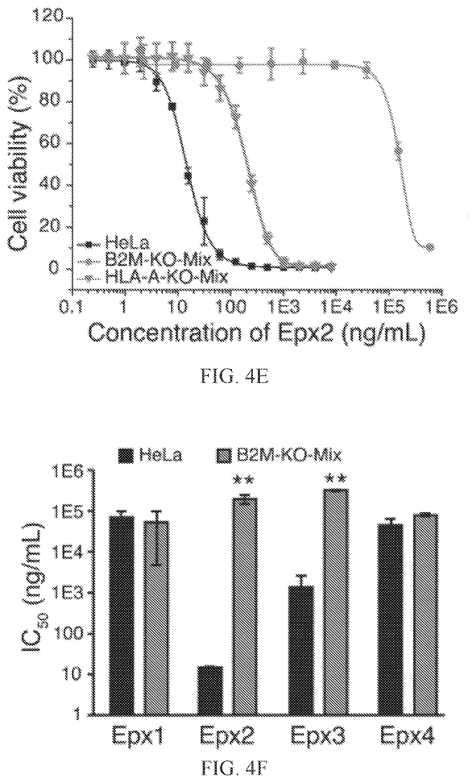

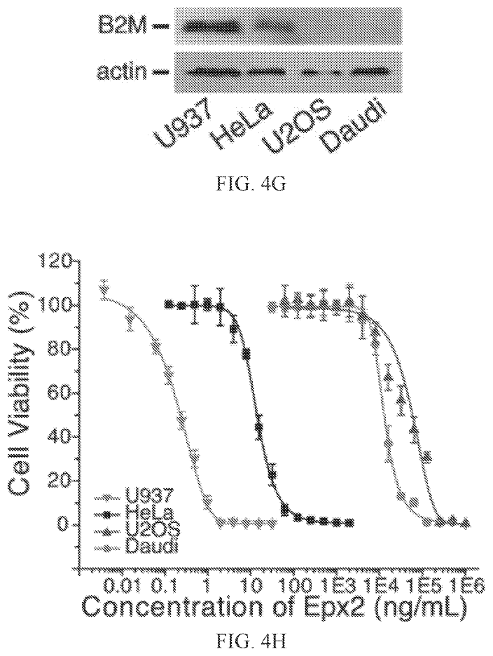

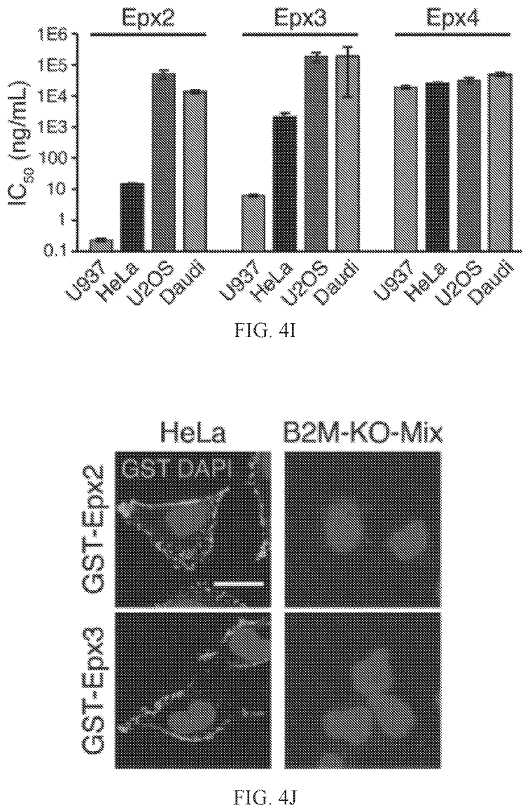

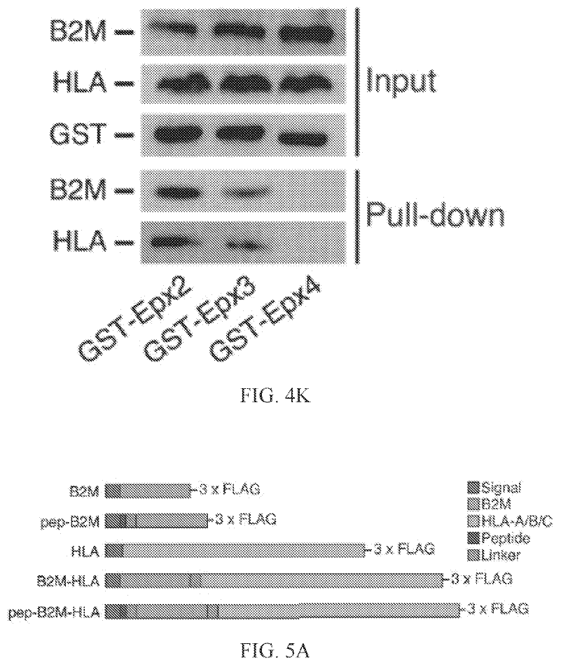

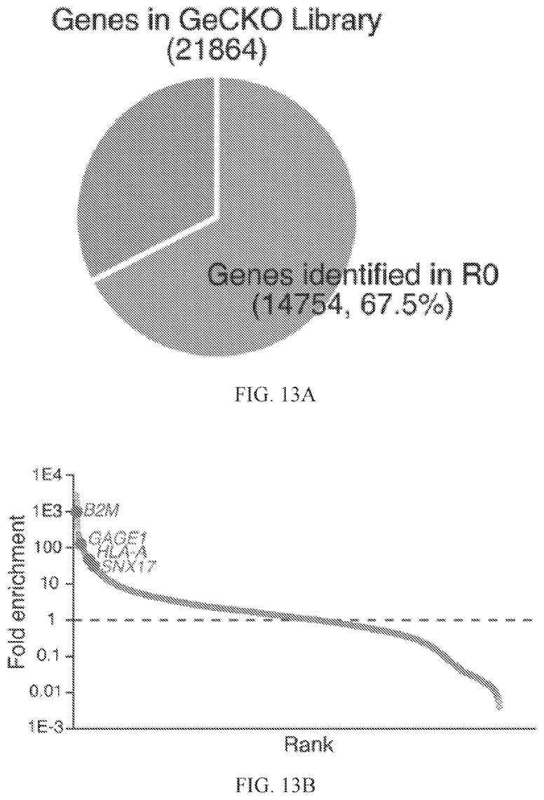

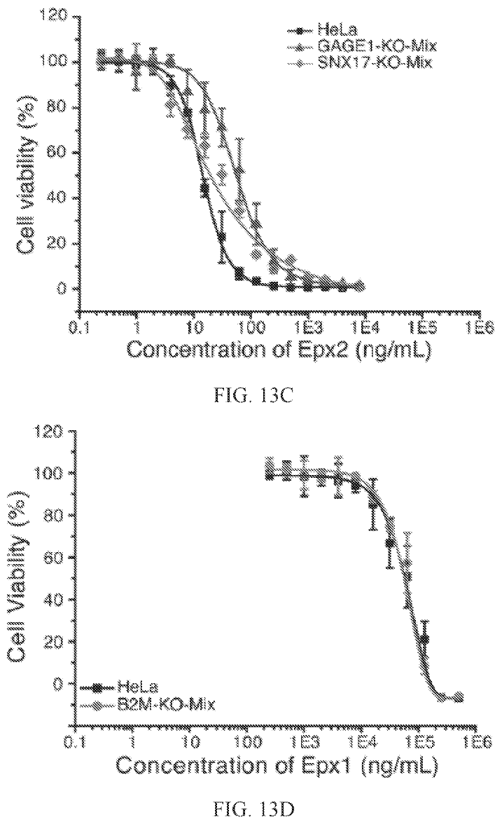

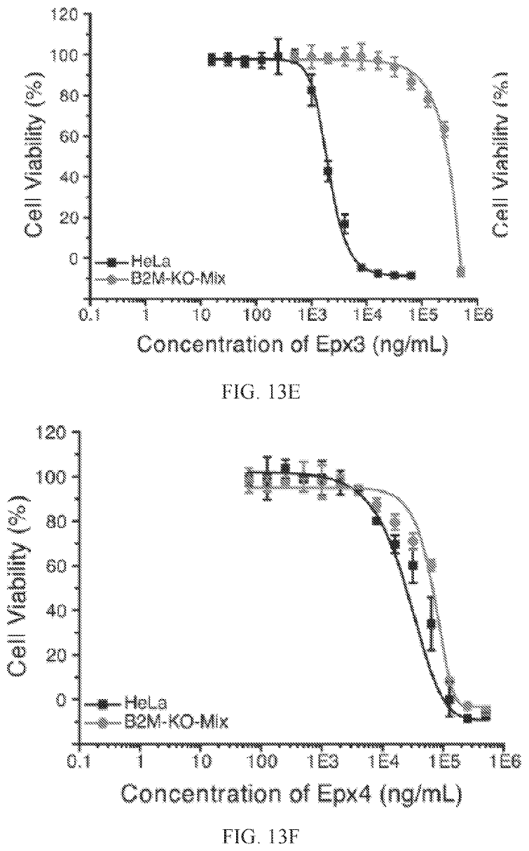

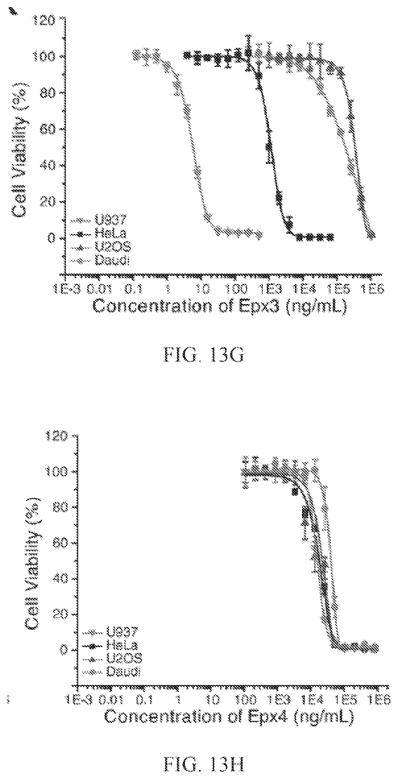

Next, receptors for Epx2 were identified using genome-wide CRISPR-Cas9 screens, as this toxin showed different toxicity levels across a range of cell lines (FIG. 10D). A genome-wide single-guide RNA (sgRNA) library (GeCKO-v2) was transduced in Hela cells that stably express Cas9 (FIG. 13A) (Sanjana et al., 2014; Tao et al., 2016; Tian et al., 2018). Cells were selected with increasing concentrations of Epx2 (FIG. 4A). The top hit was β-2-microglobulin (B2M) (FIGS. 4B and 13B). Other hits included sorting nexin-17 (SNX17) and G antigen 1 (GAGE1) (FIG. 4B).

B2M is a small protein (119 residues) that serves as the β-chain of the major histocompatibility complex class I (MHC-I, also known as HLA-I in humans) (Bjorkman et al., 1987; Neefjes et al., 2011; Pamer and Cresswell, 1998; Wieczorek et al., 2017). B2M binds to a polymorphic α-chain protein to form MHC-I/HLA-I complexes (FIG. 4C). There are three major α-chain genes (HLA-A, -B, and -C), and three minor α-chain genes (HLA-E, -F, and -G) in humans (Neefjes et al., 2011; Wieczorek et al., 2017). HLA-A was also identified in the screen, shown in FIG. 4B. All of these α-chains, which are composed of a single transmembrane domain with an extracellular domain divided into three domains (α1-3), formed a heterodimer with B2M (FIG. 4C). Binding of B2M was critical for proper trafficking of MHC/HLA-I onto cell surfaces, thus a lack of B2M would block cell surface expression of all MHC/HLA-I complexes.

To validate the top hits, stable knockout (KO) HeLa cells lacking B2M, HLA-A, SNX17, or GAGE1 were generated. B2M KO cells showed over 13,000-fold reduction in sensitivity to Epx2 compared with wild type (WT) HeLa cells (FIGS. 4D and 4E). HLA-A KO cells showed ˜14-fold reduced sensitivity to Epx2 (FIG. 4E). SNX17 and GAGE1 showed slight reduction in sensitivity but did not reach statistical significance (FIG. 13C). In addition, B2M KO cells also became resistant to Epx3, whereas their sensitivity to Epx1 and 4 was not changed (FIGS. 4F and 13A-13F).

Three human cancer cell lines were also tested, with two (U2OS and Daudi cells) that did not express detectable levels of B2M and one (U937 cells) had B2M levels higher than HeLa cells (FIG. 4G). U937 showed an IC₅₀ value of 0.2 ng/ml for Epx2 and 6 ng/ml for Epx3, ˜70 to 200-fold more sensitive than HeLa cells, whereas U2OS and Daudi cells had IC₅₀ values of over 13 μg/mL with Epx2, 3, and 4 (FIGS. 4H-4I and 13G).

Epx2 and 3 proteins fused with a glutathione S-transferase (GST) tag at their N-termini were generated. By detecting the GST tag, it was found that GST-Epx2 and -Epx3 did not bind to B2M KO cells (FIGS. 4J and 13H). Furthermore, endogenous B2M and HLA-A in HeLa cell lysates could be pulled down by GST-Epx2 and -Epx3, while GST-Epx4 showed no binding (FIG. 4K).

### Epx2 and Epx3 Recognize HLA/MHC-I Complex

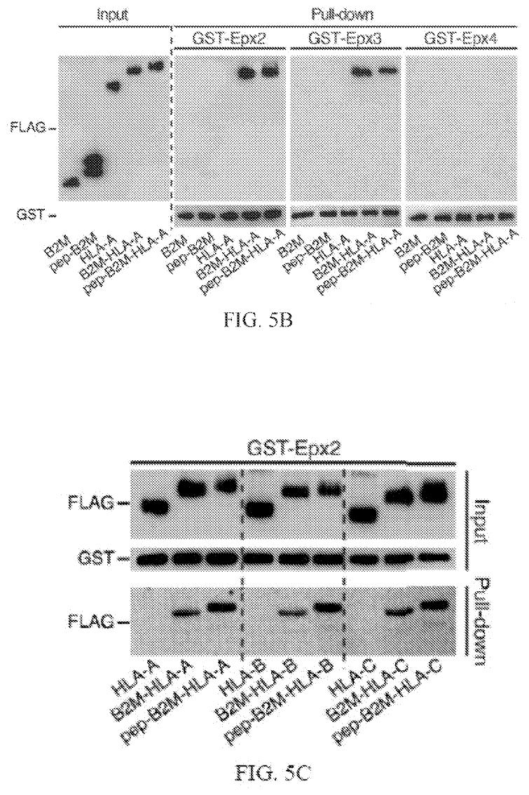

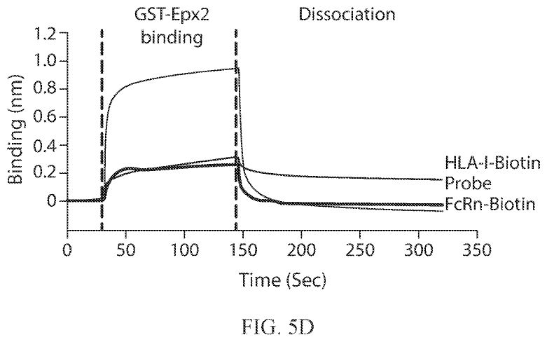

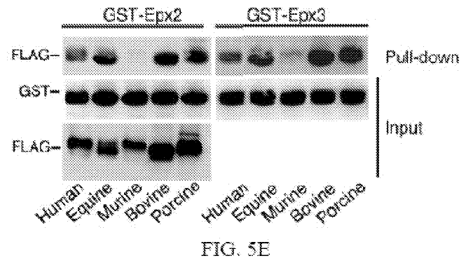

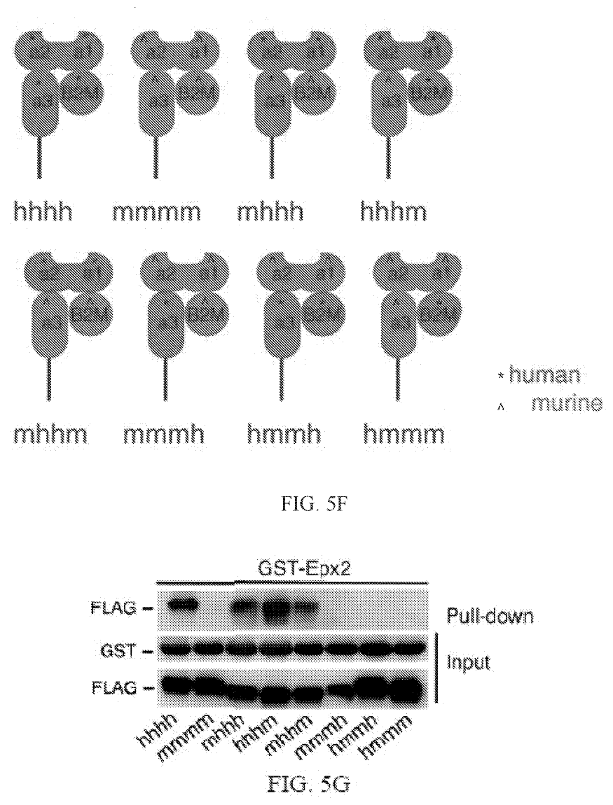

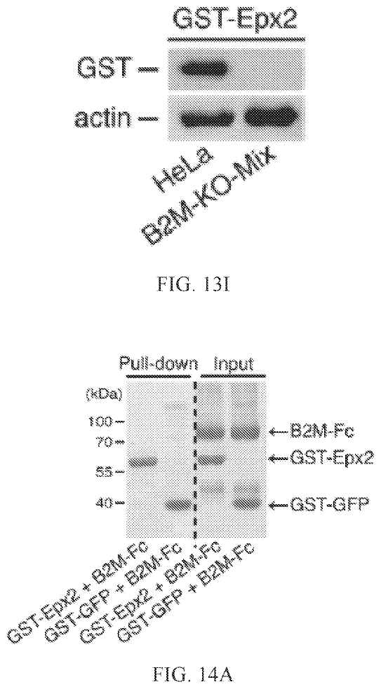

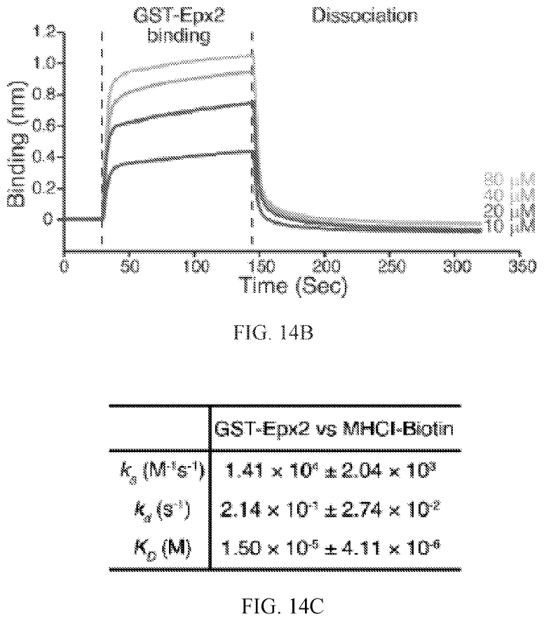

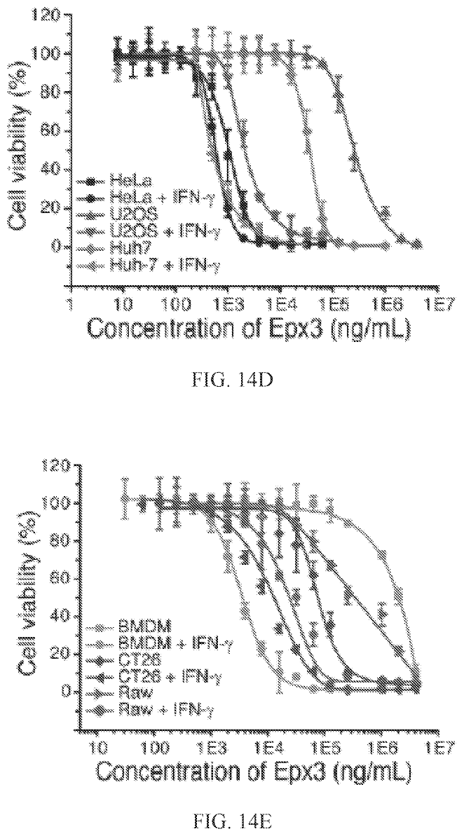

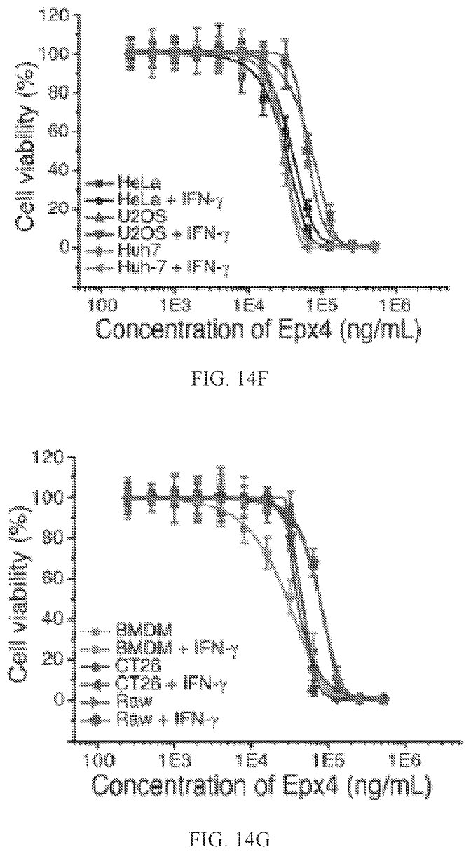

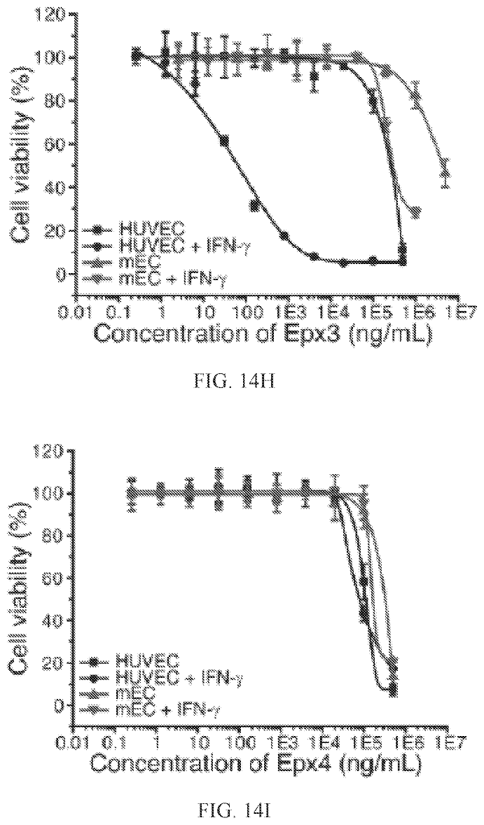

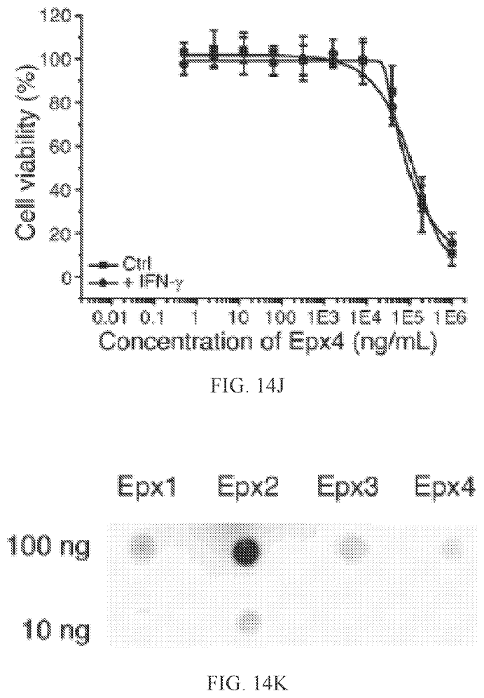

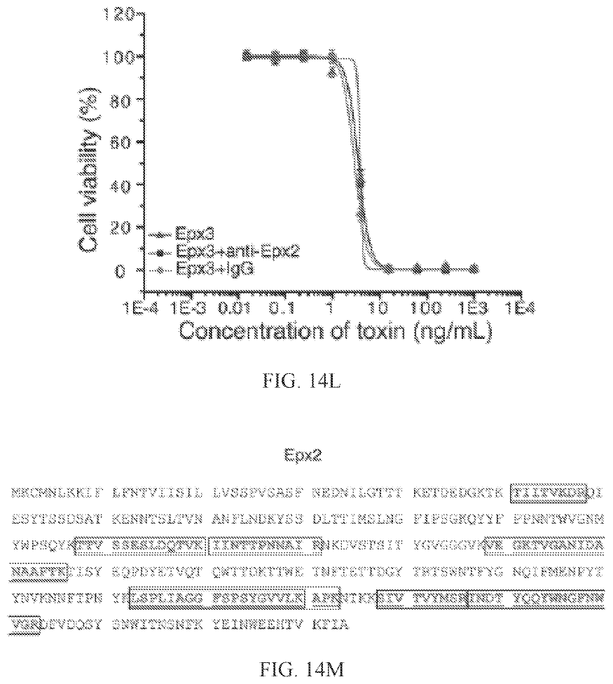

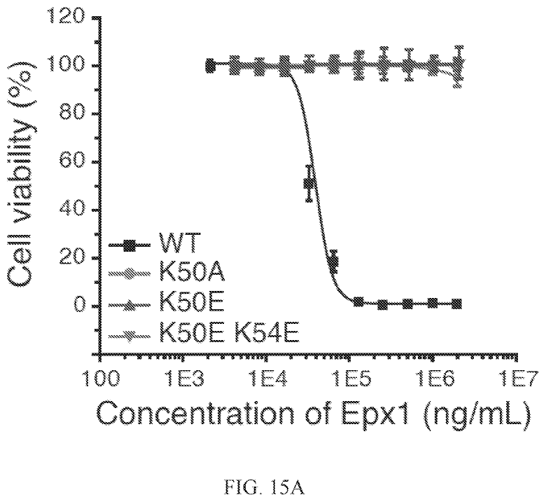

To determine whether toxins recognize B2M alone, HLA alone, or the heterodimer complex, B2M, B2M fused with an antigen peptide, HLA-A, B2M fused with HLA-A, and B2M plus a peptide fused with HLA-A were expressed in HEK293 cells (FIG. 5A). GST-Epx2 and -Epx3 pulled down HLA complexes, with or without the fused peptide, but not B2M alone or HLA-A alone from cell lysates (FIG. 5B). GST-Epx2 and -Epx3 pulled down B2M-HLA-B and B2M-HLA-C complexes as well, but not HLA-B or HLA-C alone (FIG. 5C). Consistent with these results, GST-Epx2 did not interact with purified B2M in pull-down assays (FIG. 14A).

Next, an assay was performed for direct binding of GST-Epx2 to biotinylated HLA-I complex using biolayer interferometry (FIG. 5D). GST-Epx2 did not bind to biotinylated neonatal Fc receptor complex (FcRn), despite FcRn containing B2M as a subunit (FIG. 5D) (Simister and Mostov, 1989). The binding affinity between GST-Epx2 and HLA-I complex was weak, with a K_{D} in the micromolar range (FIGS. 14B and 14C). It was possible that a proper membrane environment and multi-valent interactions were required for high-affinity binding of Epx2 on cell surfaces.

Epx2 had low activity on murine cells (FIG. 10E). Human and murine B2M and HLA-A share ˜86% and 65% sequence identity, respectively. To assess the potential species selectivity, human, mouse, equine, bovine, and porcine versions of MHC-I fused with their own B2M were expressed in HEK cells (FIG. 5E). GST-tagged Epx2 and Epx3 pulled down human, equine, bovine, and porcine MHC-I, but not murine MHC-I (FIG. 5E).

To further map the toxin binding site, domains were swapped between mouse MHC-I and HLA. The α1 and α2 domains of the HLA α-subunit formed the peptide presentation site, and the α3 domain was next to the transmembrane domain (FIG. 5F). α1-α2, α3, and B2M were switched between human and mouse versions (FIG. 5F). Pull-down assays indicated that B2M did not contain the toxin binding site. Instead, the binding site for Epx2 was located on the α1-α2 region of the HLA/MHC-I α-subunit (FIG. 5G).

### IFN-γ Sensitizes Human Cells and Intestinal Organoids to Epx2 and Epx3

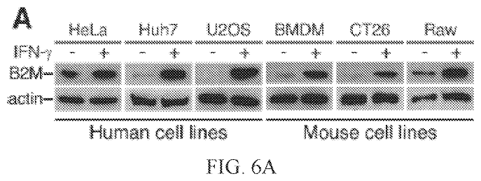

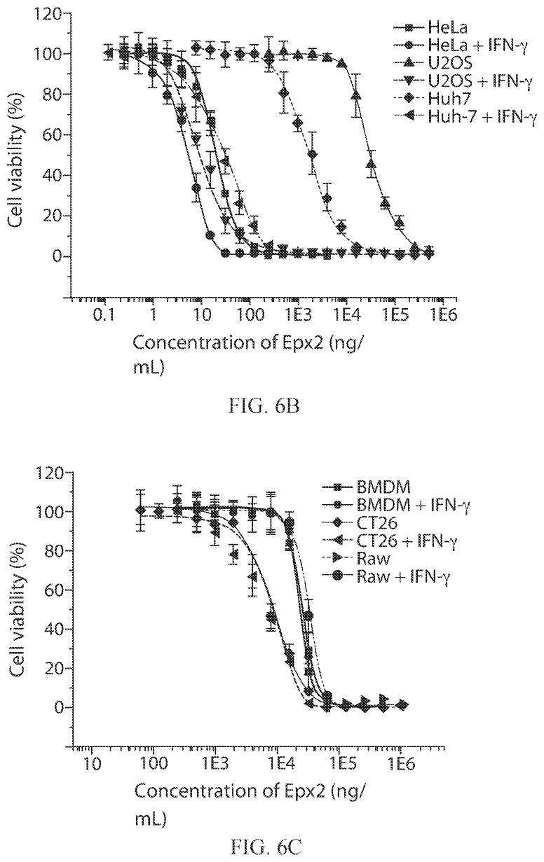

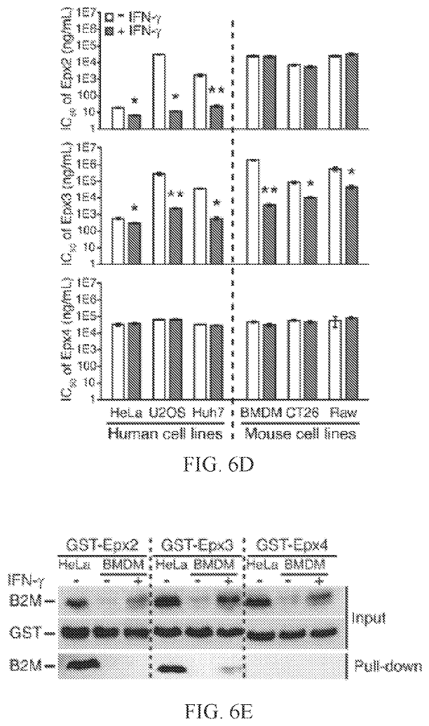

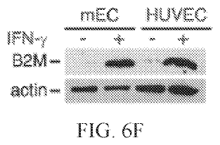

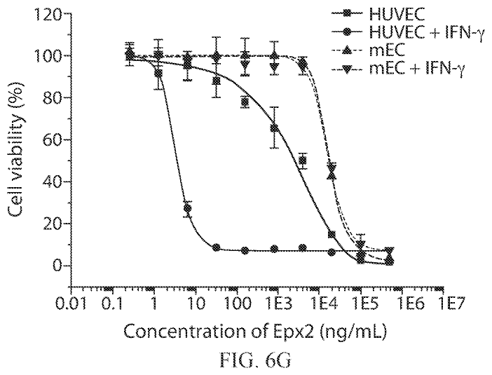

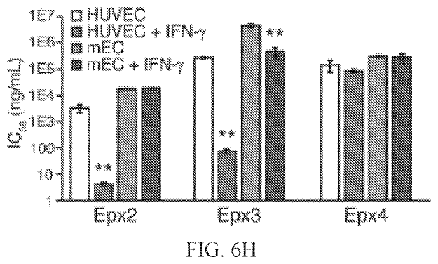

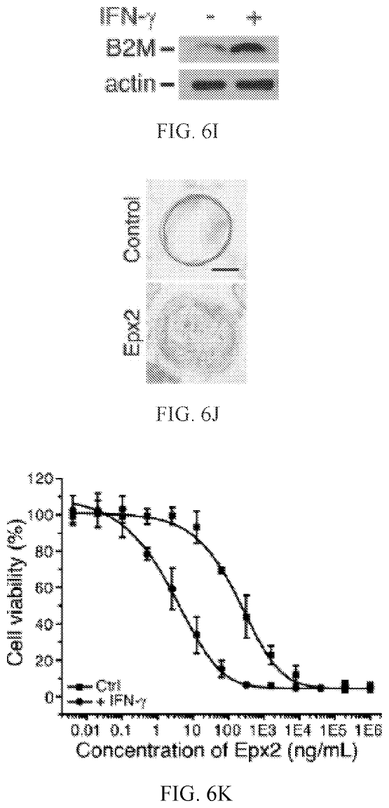

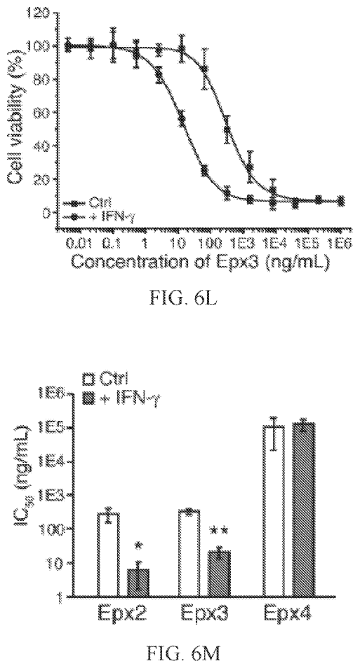

Expression of HLA/MHC-I is known to be greatly elevated by interferons (primarily IFN-γ, but also by IFN-α and -β) as part of the immune response to viral and bacterial pathogens (Fellous et al., 1982; Gough et al., 2012). The sensitivity of three human cell lines (HeLa, Huh7, and U2OS) and three murine cell lines (BMDM, CT26, and Raw) was compared to Epxs with and without pre-treatment of IFN-γ. HLA/MHC-I levels were elevated after exposure to IFN-γ (FIG. 6A). This increase rendered Huh7 cells ˜70-fold and U2OS cells >2700-fold more sensitive to Epx2, and Huh7 cells 61-fold and U2OS cells ˜118-fold more sensitive to Epx3 (FIGS. 6B, 6D, and 14D-14E). The sensitivity of HeLa cells was also increased to Epx2 and Epx3 after exposure to IFN-γ (FIGS. 6B and 6D). The sensitivities of these cells to Epx4 remained unchanged (FIGS. 6D and 14F-14G). Three murine cell lines showed no changes in their sensitivity to Epx2 and Epx4 but had increased sensitivity to Epx3 after IFN-γ treatment (FIGS. 6C-6D, 14E, and 14G). Consistent with these results, GST-Epx2 was able to pull down B2M from HeLa cells but not from BMDM (FIG. 6E), whereas Epx3 was still able to weakly pull down murine MHC-I (FIG. 6E).

To characterize the toxicity of Epx2 and Epx3 on primary cells, primary human umbilical vein endothelial cells (HUVEC) and mouse lung endothelial cells (mEC) were tested first. IFN-γ treatment increased B2M levels in HUVEC and mEC (FIG. 6F). After IFN-γ treatment, the IC₅₀ value of Epx2 with HUVEC decreased from 3.2 μg/mL to 4.2 ng/ml (819-fold), and the IC₅₀ value of Epx3 with HUVEC decreased from 268 μg/mL to 75 ng/ml (>3,500-fold) (FIGS. 6G, 6H, and 14H). Epx2 showed no change in toxicity on mEC after IFN-γ treatment (FIGS. 6G and 6H). mEC showed an increase in sensitivity to Epx3 after IFN-γ treatment, although the overall level of sensitivity remained low (FIG. 14H). The sensitivities of HUVEC and mEC to Epx4 were not changed after IFN-γ treatment (FIGS. 6H and 14I).

Next, human intestinal organoids were examined (FIG. 6I). Intestinal organoids were sensitive to Epx2 and Epx3 even without IFN-γ treatment (IC₅₀ of 277 ng/ml for Epx2 and 323 ng/mL for Epx3, FIGS. 6J-6M), and exposure to IFN-γ further enhanced their sensitivity (6 ng/mL for Epx2 and 21 ng/mL for Epx3) (FIG. 6M). Human organoids were not sensitive to Epx4 (IC₅₀ at 104 μg/mL) with or without IFN-γ treatment (FIGS. 6M and 14J).

Native Epx2 Produced by E. faecium DIV0147 is Toxic to Human Cells

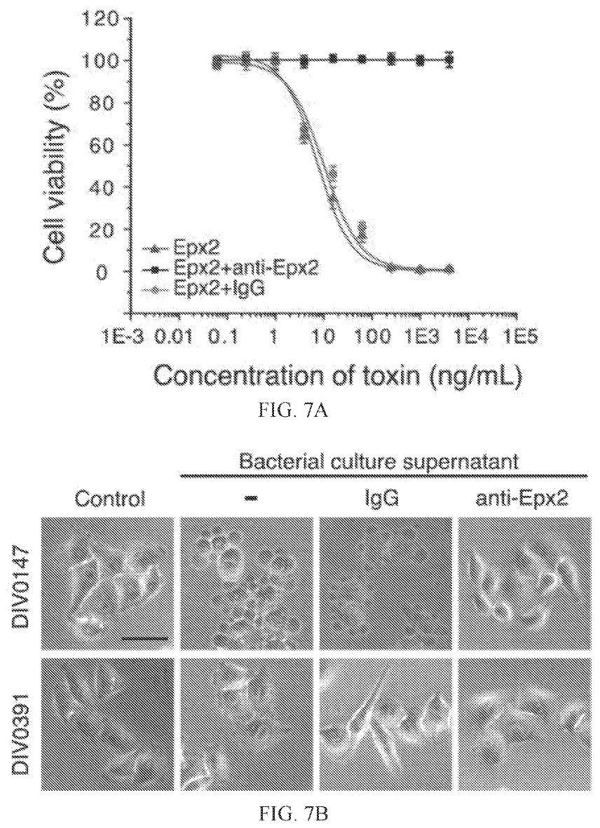

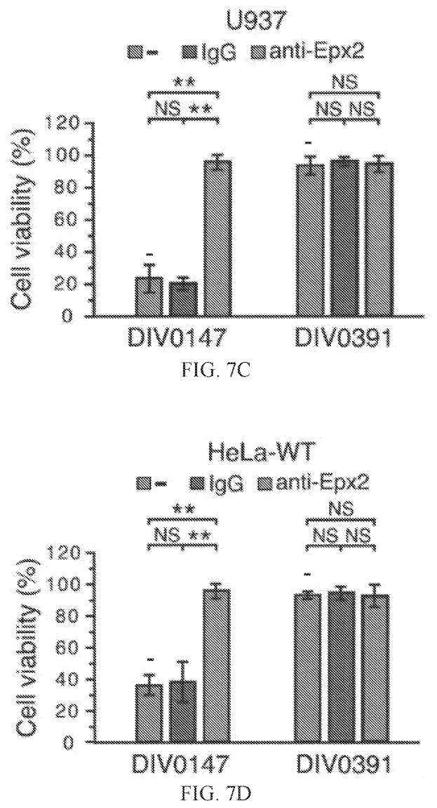

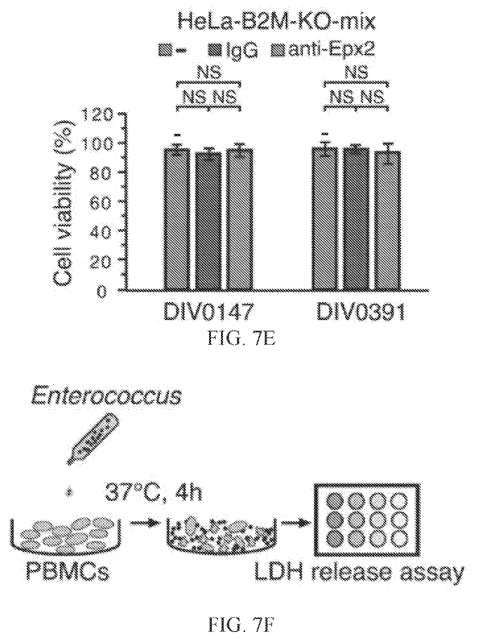

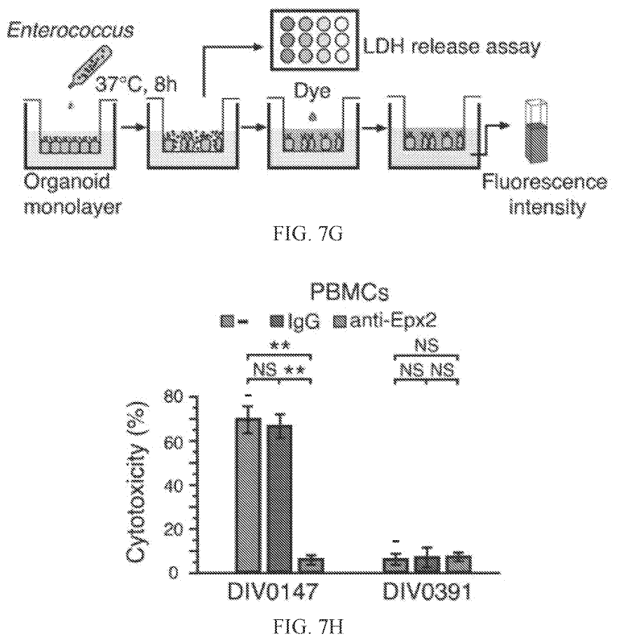

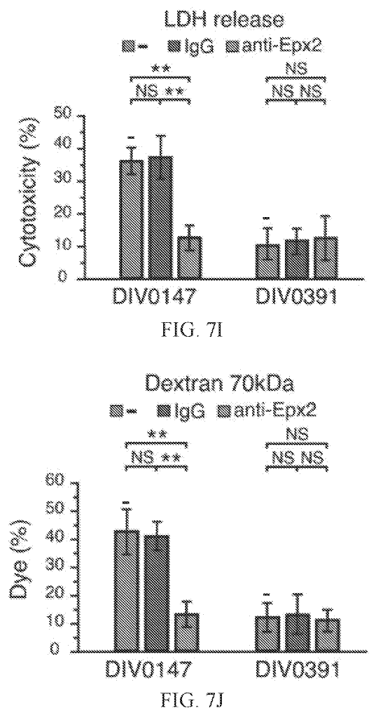

To investigate whether Epx toxins produced by Enterococcus contribute to virulence for human cells, the Epx2-carrying E. faecium strain DIV0147 was selected as a representative. A closely-related strain DIV0391, which shares ˜98.9% DNA sequence identity with DIV0147 but does not have an epx2 gene, was utilized as a control. Furthermore, a rabbit polyclonal antibody against Epx2 was produced, which could neutralize Epx2 toxicity on HeLa cells (FIG. 7A). It did not cross react with Epx1, 3, or 4, and had no effect on Epx3 toxicity on HeLa cells (FIGS. 14K and 14L).

Mass spectrometry analysis detected Epx2 directly in the supernatant of DIV0147 (FIG. 14M). The concentrated supernatant of DIV0147 induced death of HeLa cells, whereas the supernatant of the control DIV0391 strain showed no toxicity. The toxicity of DIV0147 supernatant was neutralized by the Epx2 antibody (FIG. 7B). The supernatant was concentrated in order to detect toxins, which indicated that toxin production was suppressed under lab culture conditions.

A co-culture of E. faecium bacteria with human cell lines (HeLa and U937 cells) was investigated. Co-culture with DIV0147 for 6 hours resulted in death of all cells, whereas cell viability was not affected by co-culture with the control strain (FIGS. 7C and 7D). Adding the Epx2 antibody into the medium eliminated toxicity (FIGS. 7C and 7D). Furthermore, DIV0147 showed no toxicity when co-cultured with B2M KO HeLa cells (FIG. 7E). These results demonstrated that Epx2 was solely responsible for the toxicity exhibited by DIV0147 on human cell lines.

E. faecium DIV0147 Uses Epx2 to Damage Human PBMCs and Intestinal Organoids

Immune suppression is a major function of many PFTs. Indeed, co-culture with DIV0147 damaged freshly isolated human PBMCs as measured by a lactate dehydrogenase (LDH) release assay, whereas the control strain showed no toxicity (FIG. 7F). Adding a polyclonal Epx2 antibody neutralized the toxicity of DIV0147.

Disruption of epithelial barriers is another common function of PFTs. Human intestinal organoids were cultured as monolayers in trans-wells (FIGS. 7G-7H). Co-culture experiments were carried out by adding E. faecium into the upper compartment of the trans-well. Co-culture with DIV0147 for 8 hours damaged intestinal organoid monolayers as measured by LDH release assays and disrupted cell-cell junctions as detected by increased leakage of a dextran-conjugated dye from the upper compartment into the lower compartment of the trans-well (FIGS. 7G-7H). The control strain had no toxicity and adding Epx2 antibody eliminated DIV0147 toxicity completely (FIGS. 7G-7H).

### DISCUSSION

Traits that exacerbate human infection often evolve and emerge from natural reservoirs outside of humans and historically were often not recognized until they spread into human populations. With their highly malleable genomes (Paulsen et al., 2003), enterococci can serve as a hub for inter-species gene transfer such as the transmission of vancomycin resistance to methicillin-resistant strains of S. aureus (Weigel et al., 2003). Unlike many other Gram-positive pathogens in the genera Streptococcus, Staphylococcus, and Clostridium, toxins targeting human and animal cells are rare in enterococci. Previously, a botulinum neurotoxin-like toxin was identified in a single E. faecium strain, but the targeted host species remained unknown for this toxin (Zhang et al., 2018). Here, a family of β-barrel PFTs in enterococci was identified and characterized with members highly toxic to human cells.

Functional characterization revealed the pathogenic potential of toxin-harboring E. faecium and suggest a key role of Epx2 in immune suppression and epithelial barrier disruption during pathogenesis. Besides humans, Epx2 and Epx3 can also recognize the MHC-I of major agricultural animals including horses, cattle, and pigs, which may serve as natural reservoirs for enterococci harboring these potent toxins. Considering that multi-drug resistant hospital-adapted E. faecium strains co-evolved with animal populations as they spread into humans (Lebreton et al., 2013), expansion of Epxs into strains of hospital-adapted lineages has the potential to be a devastating development.

By switching domains between human HLA-I and murine MHC-I, the binding site for Epx2 were mapped to α1-α2 domains of MHC-I, which were the polymorphic domains containing the peptide binding site for antigen presentation, and the region engaging the T-cell receptor. Binding appeared to be mediated by conserved regions on α1-α2, since Epx2 could bind to all three HLA-A, HLA-B, and HLA-C forms. Interestingly, superantigens, another class of bacterial toxins such as S. aureus toxic shock syndrome toxin 1 (TSST-1), recognized the polymorphic α1 domain of MHC-II (Jardetzky et al., 1994; Karp et al., 1990; Kim et al., 1994), which was also the domain containing the peptide binding site and engaging the T-cell receptor.

MHC-I is expressed on all nucleated cells but not on red blood cells. It presents peptide antigens derived from cytosolic proteins degraded by proteasomes. Recognition of foreign or mutated peptide antigens by CD8⁺ T cells then induces cell death to eliminate infected or cancerous cells (Neefjes et al., 2011; Wieczorek et al., 2017). The level of MHC-I is greatly increased by interferons under inflammatory conditions. IFN-γ plays a critical role in regulating both innate and adaptive host responses to not only viral infections, but also to a variety of bacterial pathogens such as S. aureus, Salmonella typhimurium, Listeria monocytogenes, and Mycobacterium tuberculosis (Shtrichman and Samuel, 2001). Commensal microbes such as Bacteroidetes induce and maintain a low level of IFN-β production from colonic dendritic cells (Abt et al., 2012; Stefan et al., 2020), which primes intestinal tissues in a vigilant state against potential viral infections. Finally, Epx2 and Epx3 may be used to induce death of virally infected cells and cancerous cells in humans in an IFN-dependent manner.

### EXPERIMENTAL MODEL AND SUBJECT DETAILS

### Cell Lines and Organoids

All the cells were cultured in DMEM media plus 10% fetal bovine serum (FBS) and 100 U penicillin/0.1 mg/mL streptomycin in a humidified atmosphere of 95% air and 5% CO₂ at 37° C.

### Bacterial Strains

Strain E. faecalis 257EA1 is derived from commercial chicken meat as reported (Manson et al., 2019). E. faecium DIV0147 was recovered from presumptive horse feces on a remote trail in Montana, USA. Culture of enterococci and purification, short read sequencing, assembly, and annotation of genomic DNA were performed essentially as described (Manson et al., 2019). E. faecium DIV0391 was isolated from crow feces in Berlin, Germany (GenBank: GCA_002141075.1)

### METHOD DETAILS

### Enterococcus Isolation, Sequencing, and Bioinformatics Analysis

Long read sequencing was performed using an Oxford Nanopore MinION™ MkI (Oxford Nanopore Technologies). Hybrid assembly of these reads with quality-trimmed 2×150 bp NextSeq™ Illumina™ reads was then performed using SPAdes 3.8.0 with default options, except for—nanopore—only-assembler—k 25,35, 45, 55, 65, 75. Scaffolds <1000 bp were removed from the assembly. Epx1 and Epx2 were first discovered using a systematic analysis of enterococci proteins of unknown function using the protein modeling and prediction tool (default parameter) Phyre2 (Kelley et al., 2015), which revealed structural homology to S. aureus leucocidin and beta and delta toxins from C. perfringens. Epx3 to 8 were discovered using blastp with Epx 1 and 3 as query sequences against the nr database with default parameters (BLOSUM62, gap existence=11, gap extension=1, with conditional compositional score matrix adjustment). Epx sequences representing all major types (1-8) were aligned in a multiple alignment using ClustalO (v1.2.1), then pairwise identity between all toxins was calculated in a 50-amino-acid sliding window across the length of the multiple alignment with a step of 1.

The 16S rRNA based phylogeny for the Enterococcus genus was extracted from the All-Species Living Tree project and edited using iTOL (Letunic and Bork, 2016). The core genome, SNP-based phylogenetic tree of E. faecalis, E. faecium, and E. hirae, was constructed using RAxML and a concatenated alignment of 1513, 1144 and 1891 single-copy core orthogroups, respectively. The 1000 bootstrap iterations were calculated using the rapid bootstrapping algorithm of RAXML. The presence of antibiotic resistance genes and plasmid predictions were determined using available online tools (PlasmidFinder and Resfinder cge.cbs.dtu.dk/services). Plasmid sequences were compared and visualized as a circular alignment using CGView (Stothard et al., 2019).

### Cell Lines and Antibodies

The following cell lines were originally obtained from ATCC: HeLa (CCL-2), A549 (CRM-CCL-185), 5637 (HTB-9A), U2OS (HTB-96), HEK293T (CRL-3216), HEK293 (CRL-1573), U937 (CRL-1593.2), Daudi (CCL-213), CT26 (CCL-2638), Raw264.7 (TIB-71), Vero (CCL-81), and MDCK (CCL-34). The Huh7 cell line was provided by Y. Matsuura. The S2 cell line was originally obtained from DGRC (RRID:CVCL_Z831). HUVEC were from pooled donors and purchased from Lonza. Anti-FLAG mouse monoclonal antibody (M2) and anti-actin mouse monoclonal antibody (AC-15) were purchased from Sigma. Mouse monoclonal antibodies against GST (8-326) were purchased from Thermo Fisher. Mouse monoclonal antibodies against HLA Class 1 ABC (ab70328) and rabbit monoclonal antibody against B2M (ab75853) were purchased from Abcam.

### Constructs

The full-length genes of Epxs were synthesized by Genewiz, with their NCBI reference numbers listed in FIG. 16. The constructs for expressing Epxs and GST-Epxs were generated by sub-cloning Epx1 (residues 24-345), Epx2 (residues 30-334), Epx3 (residues 24-329), and Epx4 (residues 32-335) into the pET22b (Addgene, 69744-3) vector with a C-terminal His6 (SEQ ID NO: 37) tag or the pGEX4T1 (Addgene, 27458001) vector with a N-terminal GST tag. Mutant Epxs were generated by site-directed mutagenesis and verified by sequencing. The cDNAs of human HLA-I were originally obtained from GE Dharmacon: B2M (MHS6278-202758740); HLA-A (MHS6278-202757462); HLA-B (MHS6278-202804742) and HLA-C (MHS1010-202726224). The full-length genes of mouse, equine, bovine, pig and rabbit B2M and HLA genes were synthesized by Twist Bioscience. The full-length B2M, full-length HLA, and B2M-HLABC fusion constructs (full length B2M with linker (GGGGS)×3 (SEQ ID NO: 34) fused with HLA-A (residues 25-365), HLA-B (residues 25-362), or HLA-C (residues 25-366), with an additional human Fc tag or a triple-FLAG tag at their C-termini (with EFGSGSGS linker (SEQ ID NO: 35))) were cloned into pcDNA3.1 vector (Invitrogen, V800-20) via Gibson Assembly (NEB, E2621). The representative antigen peptide (HER2 residues 63-71. TYLPTNASL (SEQ ID NO: 36)) and the linker (GGGGS)×3 (SEQ ID NO: 34)_were synthesized and inserted into the B2M-HLA fusion constructs via Gibson Assembly. The human and mouse HLA/MHC chimeric proteins were constructed as follows: mhhh (mB2M-(GGGGS)×3 (SEQ ID NO: 34)−hHLA no signal (25-365)), hhhm (hB2M−(GGGGS)×3 (SEQ ID NO: 34)−hHLA-A α1+α2 (25-206)−mH2K α3+TM (205-369)), mhhm (mB2M−(GGGGS)×3 (SEQ ID NO: 34)−hHLA-A α1+α2 (25-206)−mH2K α3+TM (205-369)), mmmh (mB2M−(GGGGS)×3 (SEQ ID NO: 34)−mH2K α1+α2 (22-203)−hHLA-A α3+TM (207-365)), hmmh (hB2M−(GGGGS)×3 (SEQ ID NO: 34)−mH2K α1+α2 (22-203)−hHLA−A α3+TM (207-365)), hmmm (hB2M-(GGGGS)×3 (SEQ ID NO: 34)−mH2K no signal (22-369)), with an EFGSGSGS (SEQ ID NO: 35) linker and a triple-FLAG tag at their C-termini. These constructs were cloned into pcDNA3.1 vector via Gibson Assembly.

### Protein Purification

Epxs were expressed recombinantly with either a C-terminal His6 (SEQ ID NO: 37) tag in the pET22b vector or N-terminal GST tag in pGEX4T1 vector in E. coli strain BL21 (DE3) at 20° C. for 16 hours using autoinduction medium (ForMedium AIMLB0210). Bacteria were harvested and resuspended in protein purification buffer (PP buffer) containing 200 mM NaCl, 20 mM Tris pH 7.5, 10% (v/v) glycerol, and then lysed by sonication and centrifugation. For GST-tagged proteins, bacterial lysates were applied to GSTrap columns (GE Healthcare) equilibrated with PP buffer. After washing with PP buffer, bound proteins were eluted using PP buffer containing 10 mM reduced glutathione (Sigma-Aldrich), pH 7.5. For His6 (SEQ ID NO: 37) tagged proteins, bacterial lysates were applied to HisTrap (GE Healthcare) nickel columns equilibrated with PP buffer, and columns were washed with PP buffer containing 20 mM imidazole. Bound proteins were eluted using PP buffer with a linear imidazole gradient from 20 mM to 500 mM. The proteins were further purified through HiTrap Q ion-exchange and HiLoad 16/60 Superdex 75 (GE Healthcare) gel filtration columns using the buffer containing 200 mM NaCl, 20 mM Tris pH 7.5. Proteins were concentrated using Vivaspin protein concentrator column (GE Healthcare) to ˜10 mg/mL.

### Cell Viability Assay (MTT Assay)

Cells were plated in 96-well microplates overnight to ˜70% confluence and then exposed to 2-fold serial dilutions of toxins in medium for 4 hours at 37° C. MTT (0.5 mg/mL, Research Products International M92050) was added to each well and incubated for 4 hours at 37° C. A total of 100 μL solubilization solution (10% SDS in 0.01 M HCl) was then added to each well, incubated overnight at room temperature, and the absorbance of formazan was then measured at 580 nm using a microplate reader (BMG Labtech, FLUOstar Omega). A vehicle control without toxins was analyzed in parallel. The cell viability curves were analyzed and fitted using Origin software (version 8.5). The toxin concentration that induced 50% of cells to lose viability is defined as the IC₅₀ value. Data were represented as mean±SD from three independent biological replicates. Data were considered significant when p-value <0.01 (Student's t-test, double-tail). Statistical analysis was performed using Excel.

### Liposome Leakage Assay

Liposomes were produced using POPC:PE:cholesterol at a molar ratio of 4:3:3 (Avanti polar lipids). Briefly, these lipids were dried and then rehydrated in PBS buffer together with 10 mM sulforhodamine B, incubated for 30 minutes at 37° C., followed by vigorous vertexing. The suspension was frozen in liquid nitrogen, followed by thawing at 37° C. for 5 rounds. The lipid suspension was then extruded through a 100 nm pore filter 21 times to produce liposomes, which then went through G25 desalting column to remove free dyes. The dye leakage assay was carried out by mixing toxins with 80 μL liposomes and incubating at 37° C. Sulforhodamine B release was measured every 20 s with excitation/emission wavelengths at 545/590 nm. The detergent Triton X-100 (4%, v/v) was utilized to break all liposomes to quantify the maximal signal of sulforhodamine B, which is set as 100% leakage.

### Negative Staining EM of Liposomes

Liposome-bound Epx2 samples were prepared by mixing liposome containing POPC:PE:cholesterol at a molar ratio of 4:3:3 with 2 μM Epx2 at 37° C. for 30 minutes. The formvar-carbon coated grid was placed (Electron Microscopy Sciences) with carbon side up in the Glow Discharge System at 30 mA for 30 s. 10 μL of liposome-bound Epx2 was then applied to freshly glow-discharged grid, incubated for 30 s, washed twice with H₂O and blotted by touching filter paper. The samples were then negatively stained with 2% (w/v) aqueous uranyl acetate for 1 minute and air-dried. The grids were then imaged using a Tecnai G2 Spirit BioTWIN electron microscope and recorded with an AMT 2k CCD camera.

### Oligomerization Assay

20 μL of Epxs (25 μM) proteins were mixed with 80 μL of liposomes containing POPC:PE:cholesterol at a molar ratio of 4:3:3. The mixtures were then incubated at 37° C. for 1 h. Liposome-bound Epxs were solubilized using 20 μL of protein loading buffer (375 mM Tris-HCl, 9% SDS 50% glycerol, and 0.03% bromophenol blue). Samples were analyzed by 4%-20% SDS-PAGE and Coomassie blue staining to detect SDS-resistant oligomerization bands.

### Planar Lipid Bilayer Electrophysiology

Planar lipid bilayer electrophysiology experiments were carried out using MECA chips (50 μm) on an Orbit Mini apparatus (Nanion). The lipid bilayer was painted with 50% DPhPc, 30% DOPE, and 20% DOPS at 5 mg/mL in n-octane. Purified Epx1 was added at 1 μg/mL. Once pore-formation events were detected, excess Epx1 was slowly removed by buffer exchange using a perfusion system (Eastern Scientific LLC). Experiments were carried out in mM Tris-HCl, pH 7, 100 mM NaCl and recorded at 1.25 kHz, filtered at 625 Hz and analyzed in Clampfit 10. Average channel currents were derived from three independent measurements.

### Pore Size was Calculated Using the Equation:

$$\frac{1}{γ} = ( l + \frac{π r}{2} ) \frac{ρ}{π {r}^{2}} ,$$

where γ is the pore conductance, r is the radius, l is the length of the pore (10 nm), and ρ is the resistivity of the buffer (100 Ω·cm).

### Circular Dichroism Spectroscopy

Purified wild-type Epxs and mutants were diluted to 0.5 mg/mL in PBS. Circular dichroism spectra were recorded at 20° C. using an Applied Photo-physics Chirascan plus spectropolarimeter (Jasco J-815) with a 1 mm path-length cell and a bandwidth of 1 nm. Spectra were scanned from 190 to 260 nm with a step-size of 1 nm and were repeated five times. Each reported circular dichroism curve was the average of five scans. Protein concentrations were determined with their 280 nm absorbance.

### Crystallization, Data Collection, and Structure Determination

Crystallization was performed using the sitting drop vapor diffusion method at 4° C. by mixing equal volumes (0.2-1.0 μL) of Epx4 with the reservoir solution. Crystals were grown in 5% (w/v) polyethylene glycol 8,000, 40% (v/v) MPD, 0.1 M Sodium Cacodylate, pH 6.5. Crystals were briefly soaked in cryoprotectant solution containing reservoir solution supplemented with 10% (v/v) glycerol and flash-frozen in liquid nitrogen for data collection at the Advanced Photon Source using Northeastern Collaborative Access Team (NE-CAT) beamlines 24-ID-C and 24-ID-E.

All diffraction images were indexed, integrated, and merged using HKL2000 (Otwinowski and Minor, 1997). The structure was determined by molecular replacement using MOLREP (Vagin and Teplyakov, 2010) with the delta toxin structure (PDB ID: 2YGT) (Huyet et al., 2013) as the search model. Structural refinement was carried out using PHENIX (Adams et al., 2010), and iterative model building was performed in Coot (Emsley et al., 2010). Structural FIGURES were generated using the PyMOL (pymol.org/) program. Detailed data collection and refinement statistics are provided in Table 2.

### Cryo-EM Data Collection and Analysis

A 4 μL drop of Epx1 protein at 1 mg/mL was applied to a glow-discharged Quantifoil grid (R 1.2/1.3 400 mesh, copper, Electron Microscopy Sciences) and blotted once for 6 seconds after a wait time of 15 seconds in 100% humidity at 4° C. and plunged into liquid ethane using an FEI Vitrobot Mark IV. Cryo-EM datasets were collected at 300 kV on a Titan Krios microscope (FEI) at the Harvard Cryo-Electron Microscopy Center for Structural Biology. Movies (50 frames, each 0.04 s, total dose 53.54 e/Å²) were recorded using a K3 detector (Gatan) with a defocus range of −1.5 to −2.5 μm. Automated single-particle data acquisition was performed with SerialEM, with a nominal magnification of 105,000× in counting mode, which yielded a calibrated pixel size of 0.825 Å.

Raw movies were motion-corrected using MotionCor2 (Zheng et al., 2017) and combined into micrographs, yielding 4,920 Epx1 micrographs used for image processing. The defocus value for each micrograph was determined using Gctf (Zhang, 2016). 1,963,299 particles were boxed using crYOLO (Wagner et al., 2019). Chosen particles were extracted from micrographs and binned two times (pixel size 1.65 Å) in RELION 3.1 (Zivanov et al., 2018). 2D classification was performed to discard bad particles. Good class averages were selected for the reconstruction of an initial model in RELION 3.1. 1,680,746 particles were selected for 3D classification. Cl symmetry was used for the first round of 3D classification. 800,275 particles likely representing assembly intermediates (including heptamers) and 150,842 octamer particles were selected for further processing. A round of 3D classification with C8 symmetry was performed to discard bad particles. 119,503 particles were selected for the final reconstruction. With C8 symmetry, the resolution of the Epx1 octamer map is 3.14 Å using the “gold” standard Fourier shell correlation (FSC)=0.143 criterion. After CTF refinement and Bayesian polishing, the resolution of the final map improved to 2.87 Å. DeepEMhancer was applied to improve the map's resolution at top domain (Sanchez-Garcia et al., 2021). The local resolution distribution of the map was determined by ResMap (Kucukelbir et al., 2014).

To generate an initial model, the Epx4 X-ray crystal structure was docked into the map as a rigid body using Chimera (Pettersen et al., 2004). This was followed by iterative model building in Coot (Emsley et al., 2010). PHENIX (Adams et al., 2010) was used to refine the model by iterative positional and B-factor refinement in real space.

### Genome-Wide CRISPR-Cas9 Screen and Generating KO Cells

HeLa cells that stably express Cas9 (HeLa-Cas9) were generated using lentivirus (LentiCas9-Blast, Addgene, #52962) and selected using 10 μg/mL blasticidin S (RPI, B12150.01). The GeCKO-V2 sgRNA library was obtained from Addgene (#1000000049). The sub-library A and B were independently packed into lentivirus. HeLa-Cas9 Cells were transduced with sgRNA lentiviral libraries at a MOI (multiplicity of infection) of 0.3. Infected cells were selected with 5 μg/mL puromycin (Thermo Scientific, A1113830) for one week. 3.3×10⁷ cells for sub-library A or 2.9×10⁷ cells for sub-library B were plated onto 15-cm culture dishes to ensure enough sgRNA coverage, with each sgRNA being represented 500 times. These cells were cither saved as initial library control or exposed to 0.25 μg/mL Epx for 24 h. The surviving cells were washed and re-seeded within toxin-free medium until ˜70% confluence, followed by the next round of selection with 0.5 μg/mL Epx for 24 h. The genomic DNA of surviving cells was extracted using a commercial kit (Qiagen, 13323). The DNA fragments containing the sgRNA sequences were amplified by PCR using primers lentiGP-1_F (AATGGACTATCATATGCTTACCGTAACTTGAAAGTATTTCG (SEQ ID NO: 38)) and lentiGP-3_R (ATGAATACTGCCATTTGTCTCAAGATCTAGTTACGC (SEQ ID NO: 39)). Next-generation sequencing was performed by a commercial vendor (Genewiz, Illumina HiSeq). The selected sgRNA sequences (B2M: CAGTAAGTCAACTTCAATGT (SEQ ID NO: 40); HLA-A: TCCCTCCTTACCCCATCTCA (SEQ ID NO: 41); GAGE1: GGGTCCATCTCCTGCCCATC (SEQ ID NO: 42); SNX17: CTTTCAACAGTTTCCTGCGT (SEQ ID NO: 43)) were cloned into The LentiGuide-Puro vector (Addgene, #52963). The KO cells were generated via lentiviral transduction of sgRNAs into HeLa-Cas9 cells. Mixed populations of transduced cells were selected with puromycin (5 μg/mL).

### Cell Surface Binding and Immunostaining

Cells were seeded onto glass coverslips (Hampton, HR3-239) and exposed to GST-Epx2 or GST-Epx3 (50 μg/mL) on ice for 60 minutes. Cells were washed three times with ice-cold PBS, fixed with 4% paraformaldehyde (PFA,w/v) for 20 minutes, blocked with 10% goat serum for 40 minutes, followed by incubation with primary antibodies against GST (1:500 dilution) for 1 hour and fluorescence-labeled secondary antibodies for 1 hour. Slides were scaled within DAPI-containing mounting medium (SouthernBiotech, 0100-20). Fluorescence images were captured with an Olympus DSU-IX81 spinning disk confocal system. Images were pseudo-colored and analyzed using ImageJ (Version 1.520).

### Pull-Down and Immunoblot Analysis

Cell lysates were harvested in 1 mL lysis buffer (PBS, 1% TritonX-100, 0.1% SDS, plus a protease inhibitor cocktail (Sigma-Aldrich), 1 mL per 10-cm dish) and incubated with 20 μg GST-tagged Epx proteins for 4 hours at 4° C. Pull-down experiments were carried out using 15 μL glutathione agarose beads, washed, pelleted, and boiled. Samples were subjected to SDS-PAGE analysis and transferred onto a nitrocellulose membrane (GE Healthcare, 10600002). The membrane was blocked (10 mM Tris, pH 7.4, 150 mM NaCl, 0.1% Tween-20, 5% skim milk) for 40 min, followed by incubation with primary antibodies for 1 hour and secondary antibodies for another 1 h, and then analyzed using the enhanced chemiluminescence method (Thermo Fisher Scientific, 34080). Blot images were taken using a Fuji LAS3000 imaging system. Images were analyzed using ImageJ (Version 1.520).

### Biolayer Interferometry (BLI) Assays

The binding between Epx2 and MHC-I complex was measured using the BLI assay with the BLItz system (ForteBio). Briefly, 10 μg/mL biotin-labeled human MHC-I complex (Eagle Biosciences, #1001-01) or FcRn complex (BPS Bio, #71283) were immobilized onto capture biosensors (Streptavidin (SA) Biosensors, ForteBio) and washed with DPBS containing 0.5% BSA (w/v). Empty biosensors served as a control. These biosensors were then exposed to variable concentrations of GST-Epx2 in solution (GST-Epx2 binding), followed by washing (dissociation) in DPBS (0.5% (w/v) BSA). Binding affinities (K_{D}) were estimated using the BLItz software (ForteBio). The experiments were repeated three times.

### IFN-γ Treatment

Human and mouse IFN-γ (Stemcell Technologies, #78141, #78021) powders were dissolved in PBS at a concentration of 0.1 mg/mL, and aliquoted and frozen at −20° C. Human and mouse cells were seeded into plates (96-well plates for cell viability assay and 6-well plates for immunoblots) and grew to ˜70% confluence. Human and mouse organoids were seeded in 24-well plates. IFN-γ stock was diluted 10,000-folds into culture medium at a final concentration of 10 ng/mL and cells were incubated with medium containing IFN-γ for 20 hours at 37° C. For cell viability assays, Epxs were added directly to IFN-γ-treated cells without changing medium.

Human Umbilical Vein Endothelial Cells (HUVEC) and Mouse Endothelial Cells (mEC)

HUVEC were purchased from Lonza and cultured in the 0.2% gelatin-coated plates with complete endothelial cell growth media: 40% F-12K medium (Corning), 40% DMEM medium (Corning), 20% fetal bovine serum (FBS, Thermo Fisher Scientific), 1% home-made bovine brain food, 0.2% Heparin (Sigma-Aldrich, 50 mg/mL), 1% penicillin streptomycin (Gibco), and 0.1% ciprofloxacin (Corning. 12.5 mg/mL). Primary mEC were isolated from the lungs of 8˜10-week-old C57BL/6J mice. Briefly, finely minced lung was digested with enzyme solution (2 mg/mL collagenase I, 5 mg/mL dispase, Roche) at 37° C. for 45 minutes and filtered through a 70 μm cell strainer. The suspended cells were then centrifuged at 1200 rpm at 4° C. for 8 minutes. The cell pellet was resuspended and centrifuged at 1200 rpm for 10 minutes, and the supernatant was removed. Anti-mouse CD31 MicroBeads (10 μL, Miltenyi Biotec) were added into 10⁷ cells in 90 μL of buffer (PBS, pH 7.2, 0.5% BSA, and 2 mM EDTA). The cells were then mixed and incubated for 15 minutes at 4° C. After incubation, the cells were washed with the buffer described above. Mouse CD31⁺ endothelial cells were isolated using an MS column and separator (Miltenyi Biotec), and then immediately seeded into pre-coated cell culture plates. Both HUVEC and mEC were used between passages 2 and 5.

### Human Intestinal Organoids

Cultured human intestinal organoids were provided as de-identified materials from the organoid core facility at Harvard Digestive Disease Center. These organoids are originally from de-identified endoscopic biopsy samples from pediatric patients undergoing esophagogastroduodenoscopy at Boston Children's Hospital. All methods were approved by the Institutional Review Board of Boston Children's Hospital (Protocol number IRB-P00000529). To isolate crypts, biopsies were digested in 2 mg/mL of Collagenase Type I (Life Technologies, 17018029) reconstituted in Hank's Balanced Salt Solution for 40 minutes at 37° C. Samples were then agitated by pipetting followed by centrifugation at 500 g for 5 minutes at 4° C. The crypts were resuspended in 200-300 μL of Matrigel (Corning, 356231) with 50 μL plated onto 4-6 wells of a 24-well plate and polymerized at 37° C.

Isolated crypts were grown in Matrigel with organoid growth medium, which contains (v/v): L-WRN conditioned media (50%), DMEM/F12 (45%), Glutamax (1%), N-2 supplement (1%), B-27 supplement (1%), HEPES (10 mM), primocin (100 μg/mL), normocin (100 μg/mL), A83-01 (500 nM), N-acetyl-cysteine (500 μM), recombinant murine EGF (50 ng/ml), human [Leu15]-Gastrin I (10 nM), nicotinamide (10 mM), and SB202190 (10 μM). The medium was changed every two days. After 6-8 days of culture, the medium was removed and Cell Recovery Solution (Corning, 354253) was added. The plate was incubated at 4° C. for 1 hour. The Matrigel was mechanically resuspended and centrifuged at 500 g at 4° C. for 5 minutes. The pelleted organoids were resuspended in fresh Matrigel and mechanically disrupted by pipetting up- and -down. The suspension was seeded into a fresh 24-well plate at 50 μL per well. After incubation at 37° C. for 10 minutes, 500 μL of pre-warmed organoid growth media was added. After 2 days in culture, the organoids were changed to fresh media with IFN-γ and cultured overnight. Then, serial dilutions of toxins were added to the organoids for 4 hour treatment. Cell viability was measured using the MTT assay.

### Generation of a Rabbit Polyclone Epx2 Antibody

A mutant inactive form of Epx2 (K50E/K56E, 5 mg) was purified in E. coli and utilized to immunize rabbits following a standard 3-month immunization protocol by a commercial vendor (Boston Molecules Inc., Boston, MA). Final bleeds were collected, and polyclonal antibodies were purified using a protein G column. ELISA assays were carried out to confirm the antibody titer against Epx2 (K50E/K56E). Antibodies purified with a protein G column from naïve rabbits were used as the control IgG.

Culture of E. faecium and Testing the Toxicity of Supernatants

E. faecium DIV0147 and DIV0391 were recovered from glycerol stock and grown overnight in 2 mL BHI medium (Thermo Scientific, CM1135B) 37° C. in a shaker, followed by sub-culture (1:200 dilution) in 5 mL BHI medium for 48 hours until the O.D. reached ˜2.5. Culture supernatant was collected and concentrated ˜75-fold using a protein concentrator (MilliporeSigma, UFC801008). HeLa cells were cultured in 96-well plates to ˜70% confluence. Concentrated supernatant (20 μl per well) was then added to cell culture medium (100 μl per well) and incubated for 30 minutes. Cells were then imaged using an Olympus microscope. For antibody neutralization assays, Epx2 antibody or control IgG (1 μg. 1:50 dilution) was added to each well immediately before adding the concentrated supernatant.

Co-Culture with HeLa and U937 Cells.

E. faecium DIV0147 and DIV0391 were cultured in 5 mL BHI medium for 48 hours until the O.D. reached ˜2.5. Cells were cultured in 96-well plates (˜75,000 cells per well) in standard DMEM cell culture medium (Cytiva, #SH30022) plus 10% fetal bovine serum (FBS) without antibiotics. A standard curve between O.D. and bacterial colony-forming units (CFUs) was generated by serial dilution and plating. Bacterial numbers were then quantified based on this O.D.-CFU standard curve. Bacteria were added to cell culture medium with a multiplicity of infection (MOI) at 800 and cultured together with cells for 6 hours at 37° C. Cells were washed with PBS three times and subjected to MTT assays. For antibody neutralization assays, Epx2 antibody or control IgG (2 μg, 1:25 dilution) were added to each well immediately before adding the bacteria.

Co-Culture with PBMCs and LDH Release Assays

Fresh human blood was purchased from a commercial vendor (Stemcell Technology, Cambridge, MA, #70508.2). PBMCs were isolated using a kit following supplier's instructions (Stemcell Technology, Cambridge, MA, #19654). PBMCs were seeded into a 96-well plate (˜150,000 per well) and cultured using RPMI 1640 medium (Cytiva, #SH30027) plus 2% FBS without antibiotics. IFN-γ (Stemcell Technologies, #78141, 10 ng/mL) was added to the medium. Bacteria were added to cell culture medium with a MOI at 800 and cultured together with cells for 4 hours at 37° C. Cell culture supernatants were collected and subjected to LDH release assays using a commercial kit following the manufacturer's instructions (Thermo Scientific, #C20301). LDH release from 2% Triton X-100 treatment served as a positive control and was used as 100% to normalize other measurements. For antibody neutralization assays, Epx2 antibody or control IgG (2 μg, 1:25 dilution) was added to each well immediately before adding the bacteria.

Co-Culture with Intestinal Organoids and Dye Leakage Assays

Human intestinal organoids were obtained and cultured as described above. Trans-wells (Corning, 3470) were pre-coated with 200 μL of 10% Collagen (rat tail collagen type I, 3.90 mg/mL, Corning, 354236) in PBS for 2 hours at 37° C. followed by rinsing with PBS. To seed a single trans-well, organoids from 2-4 wells of a 24-well plate were recovered from Matrigel by incubation in cell recovery solution (Corning, 354253) for 20 minutes on ice and pooled. Following centrifugation at 500 g for 5 minutes at 4° C., the pellet was resuspended in 1× TriplE Express (Gibco, #12605-010) for 10 min at 37° C. At the midpoint of this incubation, a bent P1000 tip was used to mechanically disrupt the pellet followed by pipetting up and down 50 times at the conclusion of the incubation. Chilled medium was then added to dilute the TriplE Express followed by centrifugation at 500 g for 5 minutes. The pellet was resuspended in organoid growth medium at a concentration of ˜1.5-3.0×10⁵ cells per 200 μL, seeded in pre-coated trans-wells and cultured at 37° C. for ˜7 days. Once confluent, monolayers were switched to antibiotic-free media containing 5% FBS, which contains (v/v): L-WRN conditioned media (25%), DMEM/F12 (70%), Glutamax (1%), N-2 supplement (1%), B-27 supplement (1%), HEPES (10 mM), A83-01 (500 nM), N-acetyl-cysteine (500 μM), recombinant murine EGF (50 ng/mL), human [Leu15]-Gastrin I (10 nM), nicotinamide (10 mM), and SB202190 (10 μM).

Cells were then stimulated with IFN-γ (10 ng/mL) for 16 hours. Bacterium was added to cell culture medium with MOI at 800 and co-cultured with the cells for 6 hours at 37° C. Cell culture supernatant was collected and subjected to LDH release assay. Cells were washed with PBS 3 times and subjected to permeability measurements using Rhodamine-dextran 70 kDa (Sigma, R9379). Briefly, Rhodamine-dextran was dissolved in P buffer (10 mM HEPES, pH 7.4, 1 mM sodium pyruvate, 10 mM glucose, 3 mM CaCl₂), 145 mM NaCl) or P/EGTA buffer [10 mM HEPES, pH 7.4, 1 mM sodium pyruvate, 10 mM glucose, 145 mM NaCl, 2 mM ethylene glycol-bis(β-aminoethyl ether)-N,N,N′,N′-tetraacetic acid (EGTA)].

To measure the paracellular flux, the upper, and lower cell culture media were replaced with P buffer containing Rhodamine-dextran (1 mg/ml) and P buffer alone, respectively. P/EGTA buffer containing Rhodamine-dextran (1 mg/ml) and P/EGTA buffer were used as positive controls. After incubation for 4 h, the amounts of Rhodamine-dextran in the basolateral media were measured with a fluorometer (excitation at 530 nm and emission at 590 nm). Data are expressed as fluorescent intensity. For antibody neutralization assays, Epx2 antibody or control IgG (2 μg, 1:25 dilution) was added to each well before adding the bacterium.

### Mass Spectrometry Analysis

The concentrated bacterial culture supernatants were analyzed by SDS-PAGE and Coomassie blue staining. The area around the size of Epx2 was cut into small pieces (about 1 mm×1 mm×1 mm). These gel pieces were de-stained with de-staining buffer (25 mM NH4HCO₃, 50% ACN), rinsed twice with acetonitrile, dried using speed-vac, then reduced with DTT and alkylated with iodoacetamide. Gel pieces were digested with trypsin at 37° C. overnight. Digestion was terminated by adding 1 μL of 10% trifluoroacetic acid solution, and peptides were extracted twice with extraction buffer (50% acetonitrile, 0.1% formic acid). Extracted supernatants were concentrated using speed-vac and desalted with home-made C18 stage-tips. Elution from stage-tips was dried using speed-vac and reconstituted with sample buffer (2% acetonitrile, 0.1% formic acid). Samples were then subjected to LC-MS/MS analysis.

### Quantitation and Statistical Analysis

All quantitative data were analyzed and graphed using OriginPro 9.1 software. All data are represented as mean±SD calculated using the OriginPro 9.1 software, unless indicated otherwise. Statistical details of the experiments are provided in the respective figure legends and in each methods section pertaining to the specific technique applied.

### REFERENCES

Abt, M. C., Osborne, L. C., Monticelli, L. A., Doering, T. A., Alenghat, T., Sonnenberg, G. F., Paley, M. A., Antenus, M., Williams, K. L., Erikson, J., et al. (2012). Commensal bacteria calibrate the activation threshold of innate antiviral immunity. Immunity 37, 158-170. Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta Crystallogr D Biol Crystallogr 66, 213-221.
Alonzo, F., 3rd, Kozhaya, L., Rawlings, S. A., Reyes-Robles, T., DuMont, A. L., Myszka, D. G., Landau, N. R., Unutmaz, D., and Torres, V. J. (2013). CCR5 is a receptor for Staphylococcus aureus leukotoxin ED. Nature 493, 51-55.
Arias, C. A., and Murray, B. E. (2012). The rise of the Enterococcus: beyond vancomycin resistance. Nat Rev Microbiol 10, 266-278.
Berube, B. J., and Bubeck Wardenburg, J. (2013). Staphylococcus aureus alpha-toxin: nearly a century of intrigue. Toxins (Basel) 5, 1140-1166.
Bjorkman, P. J., Saper, M. A., Samraoui, B., Bennett, W. S., Strominger, J. L., and Wiley, D. C. (1987). Structure of the human class I histocompatibility antigen, HLA-A2. Nature 329, 506-512.
Bruggisser, J., Tarek, B., Wyder, M., Muller, P., von Ballmoos, C., Witz, G., Enzmann, G., Deutsch, U., Engelhardt, B., and Posthaus, H. (2020). CD31 (PECAM-1) Serves as the Endothelial Cell-Specific Receptor of Clostridium perfringens beta-Toxin. Cell Host Microbe 28, 69-78 e66.
Coburn, P. S., Pillar, C. M., Jett, B. D., Haas, W., and Gilmore, M. S. (2004). Enterococcus faecalis senses target cells and in response expresses cytolysin. Science 306, 2270-2272.
Dal Peraro, M., and van der Goot, F. G. (2016). Pore-forming toxins: ancient, but never really out of fashion. Nat Rev Microbiol 14, 77-92.
De, S., and Olson, R. (2011). Crystal structure of the Vibrio cholerae cytolysin heptamer reveals common features among disparate pore-forming toxins. Proc Natl Acad Sci USA 108, 7385-7390.
Degiacomi, M. T., Iacovache, I., Pernot, L., Chami, M., Kudryashev, M., Stahlberg, H., van der Goot, F. G., and Dal Peraro, M. (2013). Molecular assembly of the aerolysin pore reveals a swirling membrane-insertion mechanism. Nat Chem Biol 9, 623-629.
Duan, Y., Llorente, C., Lang, S., Brandl, K., Chu, H., Jiang, L., White, R. C., Clarke, T. H., Nguyen, K., Torralba, M., et al. (2019). Bacteriophage targeting of gut bacterium attenuates alcoholic liver disease. Nature 575, 505-511.
DuMont, A. L., Yoong, P., Day, C. J., Alonzo, F., 3rd, McDonald, W. H., Jennings, M. P., and Torres, V. J. (2013). Staphylococcus aureus LukAB cytotoxin kills human neutrophils by targeting the CD11b subunit of the integrin Mac-1. Proc Natl Acad Sci USA 110, 10794-10799.
Emsley, P., Lohkamp, B., Scott, W. G., and Cowtan, K. (2010). Features and development of Coot. Acta Crystallogr D Biol Crystallogr 66, 486-501.
Fellous, M., Nir, U., Wallach, D., Merlin, G., Rubinstein, M., and Revel, M. (1982). Interferon-dependent induction of mRNA for the major histocompatibility antigens in human fibroblasts and lymphoblastoid cells. Proc Natl Acad Sci USA 79, 3082-3086.
Fiore, E., Van Tyne, D., and Gilmore, M. S. (2019). Pathogenicity of Enterococci. Microbiol Spectr 7, 10.1128.
Fluckiger, A., Daillere, R., Sassi, M., Sixt, B. S., Liu, P., Loos, F., Richard, C., Rabu, C., Alou, M. T., Goubet, A. G., et al. (2020). Cross-reactivity between tumor MHC class I-restricted antigens and an enterococci bacteriophage. Science 369, 936-942.
Freeman, V. J. (1951). Studies on the virulence of bacteriophage-infected strains of Corynebacterium diphtheriae. J Bacteriol 61, 675-688.
Gilmore, M. S., Lebreton, F., and van Schaik, W. (2013). Genomic transition of enterococci from gut commensals to leading causes of multidrug-resistant hospital infection in the antibiotic era. Curr Opin Microbiol 16, 10-16.
Goncharov, A., Grigorjev, S., Karaseva, A., Kolodzhieva, V., Azarov, D., Akhremenko, Y., Tarasova, L., Tikhonov, A., Masharskiy, A., Zueva, L., et al. (2016). Draft Genome Sequence of Enterococcus faecium Strain 58m, Isolated from Intestinal Tract Content of a Woolly Mammoth, Mammuthus primigenius. Genome Announc 4, e01706-01715.
Gough, D. J., Messina, N. L., Clarke, C. J., Johnstone, R. W., and Levy, D. E. (2012). Constitutive type I interferon modulates homeostatic balance through tonic signaling. Immunity 36, 166-174.
Guillet, V., Roblin, P., Werner, S., Coraiola, M., Menestrina, G., Monteil, H., Prevost, G., and Mourey, L. (2004). Crystal structure of leucotoxin S component: new insight into the Staphylococcal beta-barrel pore-forming toxins. J Biol Chem 279, 41028-41037.
Huyet, J., Naylor, C. E., Savva, C. G., Gibert, M., Popoff, M. R., and Basak, A. K. (2013). Structural Insights into Clostridium perfringens Delta Toxin Pore Formation. PLoS One 8, e66673.
Jardetzky, T. S., Brown, J. H., Gorga, J. C., Stern, L. J., Urban, R. G., Chi, Y. I., Stauffacher, C., Strominger, J. L., and Wiley, D. C. (1994). Three-dimensional structure of a human class II histocompatibility molecule complexed with superantigen. Nature 368, 711-718.
Karp, D. R., Teletski, C. L., Scholl, P., Geha, R., and Long, E. O. (1990). The alpha 1 domain of the HLA-DR molecule is essential for high-affinity binding of the toxic shock syndrome toxin-1. Nature 346, 474-476.
Kelley, L. A., Mezulis, S., Yates, C. M., Wass, M. N., and Sternberg, M. J. (2015). The Phyre2 web portal for protein modeling, prediction and analysis. Nat Protoc 10, 845-858.
Kim, J., Urban, R. G., Strominger, J. L., and Wiley, D. C. (1994). Toxic shock syndrome toxin-1 complexed with a class II major histocompatibility molecule HLA-DR1. Science 266, 1870-1874.
Kucukelbir, A., Sigworth, F. J., and Tagare, H. D. (2014). Quantifying the local resolution of cryo-EM density maps. Nat Methods 11, 63-65.
Lebreton, F., Manson, A. L., Saavedra, J. T., Straub, T. J., Earl, A. M., and Gilmore, M. S. (2017). Tracing the Enterococci from Paleozoic Origins to the Hospital. Cell 169, 849-861 e813.
Lebreton, F., van Schaik, W., McGuire, A. M., Godfrey, P., Griggs, A., Mazumdar, V., Corander, J., Cheng. L., Saif, S., Young, S., et al. (2013). Emergence of epidemic multidrug-resistant Enterococcus faecium from animal and commensal strains. mBio 4, e00534-00513.
Letunic, I., and Bork, P. (2016). Interactive tree of life (iTOL) v3: an online tool for the display and annotation of phylogenetic and other trees. Nucleic Acids Res 44, W242-245.
Los, F. C., Randis, T. M., Aroian, R. V., and Ratner, A. J. (2013). Role of pore-forming toxins in bacterial infectious diseases. Microbiol Mol Biol Rev 77, 173-207.
Manson, A. L., Van Tyne, D., Straub, T. J., Clock, S., Crupain, M., Rangan, U., Gilmore, M. S., and Earl, A. M. (2019). Chicken Meat-Associated Enterococci: Influence of Agricultural Antibiotic Use and Connection to the Clinic. Appl Environ Microbiol 85, e01559-01519.
Naville, M., Ghuillot-Gaudeffroy, A., Marchais, A., and Gautheret, D. (2011). ARNold: a web tool for the prediction of Rho-independent transcription terminators. RNA Biol 8, 11-13.
Neefjes, J., Jongsma, M. L., Paul, P., and Bakke, O. (2011). Towards a systems understanding of MHC class I and MHC class II antigen presentation. Nat Rev Immunol 11, 823-836.
Olson, R., Nariya, H., Yokota, K., Kamio, Y., and Gouaux, E. (1999). Crystal structure of staphylococcal LukF delineates conformational changes accompanying formation of a transmembrane channel. Nat Struct Biol 6, 134-140.
Otwinowski, Z., and Minor, W. (1997). Processing of X-ray diffraction data collected in oscillation mode. Methods Enzymol 276, 307-326.
Pamer, E., and Cresswell, P. (1998). Mechanisms of MHC class I—restricted antigen processing. Annu Rev Immunol 16, 323-358.
Parker, M. W., Buckley, J. T., Postma, J. P., Tucker, A. D., Leonard, K., Pattus, F., and Tsernoglou, D. (1994). Structure of the Aeromonas toxin proaerolysin in its water-soluble and membrane-channel states. Nature 367, 292-295.
Paulsen, I. T., Banerjei, L., Myers, G. S., Nelson, K. E., Seshadri, R., Read, T. D., Fouts, D. E., Eisen, J. A., Gill, S. R., Heidelberg, J. F., et al. (2003). Role of mobile DNA in the evolution of vancomycin-resistant Enterococcus faecalis. Science 299, 2071-2074.
Pedelacq, J. D., Maveyraud, L., Prevost, G., Baba-Moussa, L., Gonzalez, A., Courcelle, E., Shepard, W., Monteil, H., Samama, J. P., and Mourey, L. (1999). The structure of a Staphylococcus aureus leucocidin component (LukF-PV) reveals the fold of the water-soluble species of a family of transmembrane pore-forming toxins. Structure 7, 277-287.
Perelman, S. S., James, D. B. A., Boguslawski, K. M., Nelson, C. W., Ilmain, J. K., Zwack, E. E., Prescott, R. A., Mohamed, A., Tam, K., Chan, R., et al. (2021). Genetic variation of staphylococcal LukAB toxin determines receptor tropism. Nat Microbiol 6, 731-745.
Pettersen, E. F., Goddard, T. D., Huang, C. C., Couch, G. S., Greenblatt, D. M., Meng, E. C., and Ferrin. T. E. (2004). UCSF Chimera—a visualization system for exploratory research and analysis. J Comput Chem 25, 1605-1612.
Poyet, M., Groussin, M., Gibbons, S. M., Avila-Pacheco, J., Jiang, X., Kearney, S. M., Perrotta, A. R., Berdy, B., Zhao, S., Lieberman, T. D., et al. (2019). A library of human gut bacterial isolates paired with longitudinal multiomics data enables mechanistic microbiome research. Nat Med 25, 1442-1452.
Reyes-Robles, T., Alonzo, F., 3rd, Kozhaya, L., Lacy, D. B., Unutmaz, D., and Torres, V. J. (2013). Staphylococcus aureus leukotoxin ED targets the chemokine receptors CXCR1 and CXCR2 to kill leukocytes and promote infection. Cell Host Microbe 14, 453-459.
Rushton-Green, R., Darnell, R. L., Taiaroa, G., Carter, G. P., Cook, G. M., and Morgan, X. C. (2019). Agricultural Origins of a Highly Persistent Lineage of Vancomycin-Resistant Enterococcus faecalis in New Zealand. Appl Environ Microbiol 85, e00137-00119.
Sanchez-Garcia, R., Gomez-Blanco, J., Cuervo, A., Carazo, J. M., Sorzano, C. O. S., and Vargas, J. (2021). DeepEMhancer: a deep learning solution for cryo-EM volume post-processing. Commun Biol 4, 874.
Sanjana, N. E., Shalem, O., and Zhang, F. (2014). Improved vectors and genome-wide libraries for CRISPR screening. Nat Methods 11, 783-784.
Savva, C. G., Fernandes da Costa, S. P., Bokori-Brown, M., Naylor, C. E., Cole, A. R., Moss, D. S., Titball, R. W., and Basak, A. K. (2013). Molecular architecture and functional analysis of NetB. a pore-forming toxin from Clostridium perfringens. J Biol Chem 288, 3512-3522.
Shtrichman, R., and Samuel, C. E. (2001). The role of gamma interferon in antimicrobial immunity. Curr Opin Microbiol 4, 251-259.
Simister, N. E., and Mostov, K. E. (1989). An Fc receptor structurally related to MHC class I antigens. Nature 337, 184-187.
Song. L., Hobaugh, M. R., Shustak, C., Cheley, S., Bayley, H., and Gouaux. J. E. (1996). Structure of staphylococcal alpha-hemolysin, a heptameric transmembrane pore. Science 274, 1859-1866.
Spaan, A. N., Henry, T., van Rooijen, W. J. M., Perret, M., Badiou, C., Aerts, P. C., Kemmink, J., de Haas, C. J. C., van Kessel, K. P. M., Vandenesch, F., et al. (2013). The staphylococcal toxin Panton-Valentine Leukocidin targets human C5a receptors. Cell Host Microbe 13, 584-594.
Spaan, A. N., van Strijp, J. A. G., and Torres, V. J. (2017). Leukocidins: staphylococcal bi-component pore-forming toxins find their receptors. Nat Rev Microbiol 15, 435-447.
Stefan, K. L., Kim, M. V., Iwasaki, A., and Kasper, D. L. (2020). Commensal Microbiota Modulation of Natural Resistance to Virus Infection. Cell 183, 1312-1324 e1310.
Stothard, P., Grant, J. R., and Van Domselaar, G. (2019). Visualizing and comparing circular genomes using the CGView family of tools. Brief Bioinform 20, 1576-1582.
Sugawara, T., Yamashita, D., Kato, K., Peng, Z., Ueda, J., Kaneko, J., Kamio, Y., Tanaka, Y., and Yao, M. (2015). Structural basis for pore-forming mechanism of staphylococcal alpha-hemolysin. Toxicon 108, 226-231.
Tanaka, Y., Hirano, N., Kaneko, J., Kamio, Y., Yao, M., and Tanaka, I. (2011). 2-Methyl-2,4-pentanediol induces spontaneous assembly of staphylococcal alpha-hemolysin into heptameric pore structure. Protein Sci 20, 448-456.
Tao, L., Zhang, J., Meraner, P., Tovaglieri, A., Wu, X., Gerhard, R., Zhang. X., Stallcup, W. B., Miao. J., He. X., et al. (2016). Frizzled proteins are colonic epithelial receptors for C. difficile toxin B. Nature 538, 350-355.
Tian, S., Muneeruddin, K., Choi, M. Y., Tao, L., Bhuiyan, R. H., Ohmi, Y., Furukawa, K., Boland, S., Shaffer, S. A., Adam, R. M., et al. (2018). Genome-wide CRISPR screens for Shiga toxins and ricin reveal Golgi proteins critical for glycosylation. PLOS Biol 16, e2006951.
Tromp, A. T., Van Gent, M., Abrial, P., Martin, A., Jansen, J. P., De Haas, C. J. C., Van Kessel,
K. P. M., Bardoel, B. W., Kruse, E., Bourdonnay, E., et al. (2018). Human CD45 is an F-component-specific receptor for the staphylococcal toxin Panton-Valentine leukocidin. Nat Microbiol 3, 708-717.
Tyson, G. H., Sabo, J. L., Hoffmann, M., Hsu, C. H., Mukherjee, S., Hernandez, J., Tillman, G., Wasilenko, J. L., Haro, J., Simmons, M., et al. (2018). Novel linezolid resistance plasmids in Enterococcus from food animals in the USA. J Antimicrob Chemother 73, 3254-3258.
Vagin, A., and Teplyakov, A. (2010). Molecular replacement with MOLREP. Acta Crystallogr D Biol Crystallogr 66, 22-25.
Van Tyne, D., and Gilmore, M. S. (2014). Friend turned foe: evolution of enterococci virulence and antibiotic resistance. Annu Rev Microbiol 68, 337-356.
Van Tyne, D., Martin, M. J., and Gilmore, M. S. (2013). Structure, function, and biology of the Enterococcus faecalis cytolysin. Toxins (Basel) 5, 895-911.
Wagner, T., Merino, F., Stabrin, M., Moriya, T., Antoni, C., Apelbaum, A., Hagel, P., Sitsel, O., Raisch, T., Prumbaum, D., et al. (2019). SPHIRE-crYOLO is a fast and accurate fully automated particle picker for cryo-EM. Commun Biol 2, 218.
Weigand, M. R., Ashbolt, N. J., Konstantinidis, K. T., and Santo Domingo, J. W. (2014). Genome sequencing reveals the environmental origin of enterococci and potential biomarkers for water quality monitoring. Environ Sci Technol 48, 3707-3714.
Weigel, L. M., Clewell, D. B., Gill, S. R., Clark, N. C., McDougal, L. K., Flannagan, S. E., Kolonay, J. F., Shetty, J., Killgore, G. E., and Tenover, F. C. (2003). Genetic analysis of a high-level vancomycin-resistant isolate of Staphylococcus aureus. Science 302, 1569-1571.
Wieczorek, M., Abualrous, E. T., Sticht, J., Alvaro-Benito, M., Stolzenberg, S., Noe, F., and Freund, C. (2017). Major Histocompatibility Complex (MHC) Class I and MHC Class II Proteins: Conformational Plasticity in Antigen Presentation. Front Immunol 8, 292.
Wilke, G. A., and Bubeck Wardenburg, J. (2010). Role of a disintegrin and metalloprotease 10 in Staphylococcus aureus alpha-hemolysin-mediated cellular injury. Proc Natl Acad Sci USA 107, 13473-13478.
Yamashita, D., Sugawara, T., Takeshita, M., Kaneko, J., Kamio, Y., Tanaka, I., Tanaka, Y., and Yao, M. (2014). Molecular basis of transmembrane beta-barrel formation of staphylococcal pore-forming toxins. Nat Commun 5, 4897.
Yamashita, K., Kawai, Y., Tanaka, Y., Hirano, N., Kaneko, J., Tomita, N., Ohta, M., Kamio, Y., Yao, M., and Tanaka, I. (2011). Crystal structure of the octameric pore of staphylococcal gamma-hemolysin reveals the beta-barrel pore formation mechanism by two components. Proc Natl Acad Sci USA 108, 17314-17319.
Yan, X. X., Porter, C. J., Hardy, S. P., Steer, D., Smith, A. I., Quinsey, N. S., Hughes, V., Cheung, J. K., Keyburn, A. L., Kaldhusdal, M., et al. (2013). Structural and functional analysis of the pore-forming toxin NetB from Clostridium perfringens. mBio 4, e00019-00013.
Zaheer, R., Cook, S. R., Barbieri, R., Goji, N., Cameron, A., Petkau, A., Polo, R. O., Tymensen, L., Stamm, C., Song, J., et al. (2020). Surveillance of Enterococcus spp. reveals distinct species and antimicrobial resistance diversity across a One-Health continuum. Sci Rep 10, 3937.
Zhang, K. (2016). Gctf: Real-time CTF determination and correction. J Struct Biol 193, 1-12.
Zhang, S., Lebreton, F., Mansfield, M. J., Miyashita, S. I., Zhang, J., Schwartzman, J. A., Tao, L., Masuyer, G., Martinez-Carranza, M., Stenmark, P., et al. (2018). Identification of a Botulinum Neurotoxin-like Toxin in a Commensal Strain of Enterococcus faecium. Cell Host Microbe 23, 169-176 e166.
Zheng, S. Q., Palovcak, E., Armache, J. P., Verba, K. A., Cheng, Y., and Agard, D. A. (2017). MotionCor2: anisotropic correction of beam-induced motion for improved cryo-electron microscopy. Nat Methods 14, 331-332.
Zivanov, J., Nakane, T., Forsberg, B. O., Kimanius, D., Hagen, W. J., Lindahl, E., and Scheres, S. H. (2018). New tools for automated high-resolution cryo-EM structure determination in RELION-3. Elife 7, e42166.

What is claimed is:

1. An isolated Enterococci toxin (Epx) polypeptide, comprising an amino acid sequence that is at least 85% identical to any one of SEQ ID NOs: 1-8, wherein the amino acid sequence is not 100% identical to any one of SEQ ID NOs: 1-8.

2. The isolated Epx polypeptide of claim 1, comprising an amino acid sequence that is at least 95% identical to any one of SEQ ID NOs: 1-8.

3. The isolated Epx polypeptide of claim 1 or claim 2, further comprising a signal sequence.

4. The isolated Epx polypeptide of claim 3, wherein the signal sequence is selected from the group consisting of SEQ ID NOs: 26-33.

5. A nanopore comprising the isolated Epx polypeptide of any one of claim 1-4.

6. An apparatus comprising the nanopore of claim 5 and a membrane.

7. The apparatus of claim 6, wherein the nanopore is disposed in the membrane.

8. A modified Epx polypeptide, comprising an amino acid sequence that is at least 85% identical to any one of SEQ ID NOs: 9-16 and an amino acid substitution at a position corresponding to K50 and/or K56 of SEQ ID NO: 9.

9. The modified Epx polypeptide of claim 8, comprising an amino acid sequence that is at least 95% identical to any one of SEQ ID NOs: 9-16 and an amino acid substitution at a position corresponding to K50 and/or K56 of SEQ ID NO: 9.

10. The modified Epx polypeptide of claim 8, comprising of the amino acid sequence of any one of SEQ ID NOs: 9-16 and an amino acid substitution at a position corresponding to K50 and/or K56 of SEQ ID NO: 9.

11. The modified Epx polypeptide of any one of claim 8-10, wherein the amino substitution introduces a neutral amino acid or a negatively charged amino acid.

12. The modified Epx polypeptide of any one of claim 8-10, wherein the amino acid substitution corresponds to K50E or K50A of SEQ ID NO: 9.

13. The modified Epx polypeptide of any one of claim 8-10, wherein the amino acid substitution corresponds to K54E or K54A of SEQ ID NO: 9.

14. The modified Epx polypeptide of any one of claim 8-10, comprising an amino acid substitution corresponding to K50E or K50A of SEQ ID NO: 9, and an amino acid substitution corresponding to K56E or K56A of SEQ ID NO: 9.

15. The modified Epx polypeptide of claim 8 comprising an amino acid sequence that is at least 85% identical to of any one of SEQ ID NOs: 17-25.

16. The modified Epx polypeptide of claim 8 comprising an amino acid sequence that is at least 95% identical to of any one of SEQ ID NOs: 17-25.

17. The modified Epx polypeptide of claim 8 comprising an amino acid sequence of any one of SEQ ID NOs: 17-25.

18. A composition comprising the modified Exp polypeptide of any one of claim 8-17, or a fragment thereof.

19. The composition of claim 18, further comprising an antigen.

20. The composition of claim 19, wherein the antigen is a viral antigen, a bacterial antigen, a cancer antigen, a fungal antigen, or a parasitic antigen.

21. The composition of claim 19 or claim 20, wherein the antigen in a peptide antigen.

22. The composition of any one of claim 19-21, wherein the antigen is conjugated to the Exp polypeptide.

23. The composition of claim 22, wherein the antigen is a peptide antigen fused to the Exp polypeptide, forming a fusion protein.

24. The composition of claim 23, wherein the fusion protein from N-terminal to C-terminal comprises the peptide antigen then the modified Epx polypeptide or a fragment thereof.

25. The composition of claim 23, wherein the fusion protein from N-terminal to C-terminal comprises the n-terminal of the modified Epx polypeptide or a fragment thereof then the peptide antigen.

26. The composition of claim 18, wherein the composition is an immunogenic composition.

27. The composition of any one of claim 19-25, wherein the composition is an immunogenic composition.

28. The composition of claim 26 or claim 27, wherein the immunogenic composition is a vaccine.

29. The composition of claim 27 or claim 28, wherein the modified Exp polypeptide is used as an adjuvant.

30. A method of inducing an immune response against Enterococci in a subject, the method comprising administering to the subject a modified Exp polypeptide of any one of claim 8-17 or the composition of any one of claim 18-29.

31. The method of claim 30, wherein the Enterococci is a multi-drug resistant Enterococci.

32. A method of inducing an immune response against an antigen in a subject, the method comprising administering to the subject the composition of any one of claim 19-25 and 26-29.

33. The method of any one of claim 30-32, wherein the method is therapeutic.

34. The method of any one of claim 30-32, wherein the method is prophylactic.

35. The method of any one of claim 30-34, wherein the subject is a mammalian subject.

36. The method of any one of claim 30-35, wherein the subject is a human subject.

37. A method of blocking MHC class I activity, the method comprising contacting an MHC class I receptor with the isolated Epx polypeptide of any one of claim 1-4, the modified Epx polypeptide of any one of claim 8-17, or the composition of any one of claim 18-29.

38. The method of claim 37, wherein the contacting occurs in a cell free assay.

39. The method of claim 37, wherein the contacting occurs in in vitro cell culture.

40. The method of claim 37, wherein the contacting occurs in a subject.

41. The method of claim 40, wherein the subject is an animal.

42. The method of claim 40, wherein the subject is a human.

43. The method of claim 37, wherein the epx polypeptide binds to a α1-α2 region of the MHC class I α-subunit.

44. A method of treating a disease associated with detrimental MHC class I activity, the method comprising administering to a subject the isolated Epx polypeptide of any one of claim 1-4, the modified Epx polypeptide of any one of claim 8-17, or the composition of any one of claim 18-29.

45. The method of claim 44, wherein the disease is selected from the group consisting of cancer, an autoimmune disease, a bacterial infection, a viral infection, a parasitic infection, or a fungal infection.

46. The method of claim 44 or claim 45, wherein the disease is selected from the group consisting of idiopathic inflammatory muscle diseases, diabetes, chronic inflammation, rheumatoid Arthritis, ankylosing spondylitis, asthma, Alzheimer's disease, Inflammatory bowel disease, obesity, Fatty liver disease and Endometriosis.

47. The method of claim 44 or claim 45, wherein the subject is a mammalian subject.

48. The method of claim 44 or claim 45, wherein the subject is a human subject.

49. A nucleic acid sequence encoding the isolated Epx polypeptide of any one of claim A1-A4, the modified Epx polypeptide of any one of claim 8-17, or the fusion protein of any one of claim 23-25.

50. A vector comprising the nucleic acid sequence of claim 49.

51. The vector of claim 50, wherein the vector is a plasmid.

52. A cell comprising the isolated Epx polypeptide of any one of claim 1-4, the modified Epx polypeptide of any one of claim 8-17, or the fusion protein of any one of claim 23-25, the nucleic acid sequence of claim 49, or the vector of claim 50 or claim 51.

53. The cell of claim 52, wherein the cell is a bacterial cell.

54. The cell of claim 53, wherein the cell is an Enterococci cell.

55. The cell of claim 52, wherein the cell is a mammalian cell.

56. A method of producing an Exp polypeptide, the method comprising culturing the cell of any one of claim 52-55 under conditions that that permits expression of the Exp polypeptide.

57. The method of claim 56, further comprising isolating the Exp polypeptide.
